SEQUENCE LISTING (12 sequences)

SEQ ID NO: 1 - DNA, 20 bp, Artificial Sequence
  ACTB - Forward
ctcttccagccttccttcct

SEQ ID NO: 2 - DNA, 20 bp, Artificial Sequence
  ACTB - Reverse
agcactgtgttggcgtacag

SEQ ID NO: 3 - DNA, 20 bp, Artificial Sequence
  BMP4 - Forward
aagcgtagccctaagcatca

SEQ ID NO: 4 - DNA, 20 bp, Artificial Sequence
  BMP4 - Reverse
tggttgagttgaggtggtca

SEQ ID NO: 5 - DNA, 20 bp, Artificial Sequence
  KIMI - Forward
cgacaacgactgttccaatg

SEQ ID NO: 6 - DNA, 20 bp, Artificial Sequence
  KIMI - Reverse
aaaggcattggaggaacaaa

SEQ ID NO: 7 - DNA, 21 bp, Artificial Sequence
  MIXLI - Forward
acgtctttcagcgccgaacag

SEQ ID NO: 8 - DNA, 21 bp, Artificial Sequence
  MIXLI - Reverse
ttggttcgggcaggcagttca

SEQ ID NO: 9 - DNA, 20 bp, Artificial Sequence
  OSRI - Forward
ccttccttcaggcagtgaac

SEQ ID NO: 10 - DNA, 20 bp, Artificial Sequence
  OSRI - Reverse
cggcactttggagaaagaag

SEQ ID NO: 11 - DNA, 20 bp, Artificial Sequence
  PAX2 - Forward
actccatcaatgggatcctg

SEQ ID NO: 12 - DNA, 20 bp, Artificial Sequence
  PAX2 - Reverse
ccacaccactctgggaatct

(19) United States
(12) Patent Application Publication
(10) Pub. No.: US 2018/0258404 A1
(43) Pub. Date: Sep. 13, 2018

(54) METHODS OF GENERATING NEPHRONS FROM HUMAN PLURIPOTENT STEM CELLS

(71) Applicant: THE BRIGHAM AND WOMEN'S HOSPITAL, INC., Boston, MA (US)

(72) Inventors: Joseph V. Bonventre, Wayland, MA (US); Ryuji Morizane, Brookline, MA (US)

(73) Assignee: THE BRIGHAM AND WOMEN'S HOSPITAL, INC., Boston, MA (US)

(21) Appl. No.: 15/760,361

(22) PCT Filed: Sep. 16, 2016
(86) PCT No.: PCT/US2016/052350

§ 371 Date: Mar. 15, 2018

Related U.S. Application Data
(60) Provisional application No. 62/219,704, filed on Sep. 17, 2015.

(51) Int. Cl. C12N 5/071

(52) U.S. Cl. CPC C12N 5/0686; C12N 2506/02; C12N 2501/119; C12N 2501/155

(57) ABSTRACT

The Inventors established an efficient, chemically defined protocol for differentiating hPSCs into multi-potent nephron progenitor cells (NPCs) that can form nephron-like structures. By recapitulating metanephric kidney development in vitro, the Inventors generate SIX2+SALL1+WT1+PAX2+ NPCs with 90% efficiency within 9 days of differentiation. The NPCs possess the developmental potential of their in vivo counterparts and form PAX8+LHX1+ renal vesicles that self-pattern into nephron structures. In both 2D and 3D culture, NPCs form kidney organoids containing epithelial nephron-like structures expressing markers of podocytes, proximal tubules, loops of Henle, and distal tubules in an organized, continuous arrangement that resembles the nephron in vivo. The Inventors also show that this organoid culture system can be used to study mechanisms of human kidney development and toxicity.

### FIELD OF THE INVENTION

Descried herein are methods and compositions related to production of nephron progenitor cells and kidney organoids. The techniques described herein find use in regenerative medicine applications.Human stem cells can be cultured in three-dimensional cultures recapitulate tissue-specific epithelial morphogenesis, physiology, and disease.

### BACKGROUND

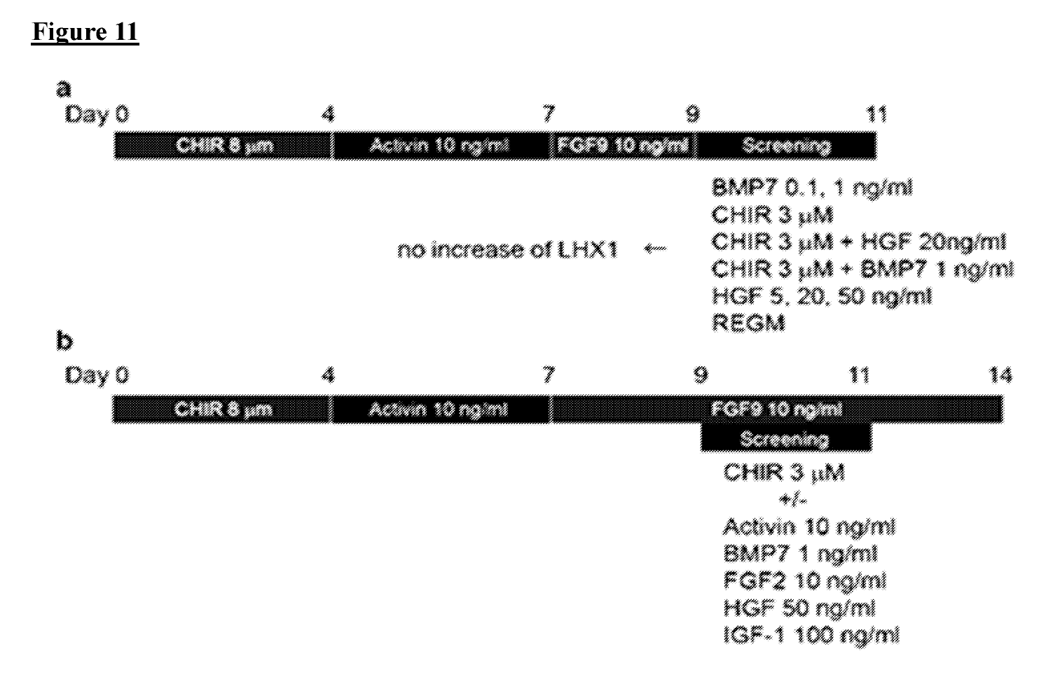

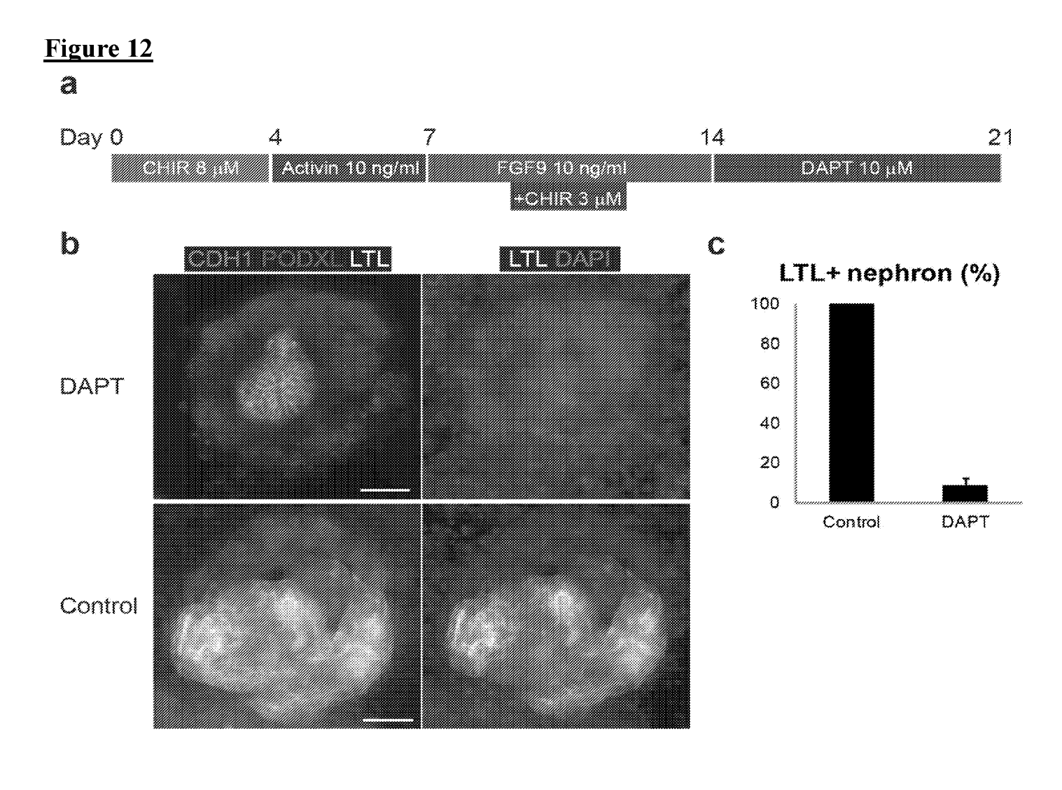

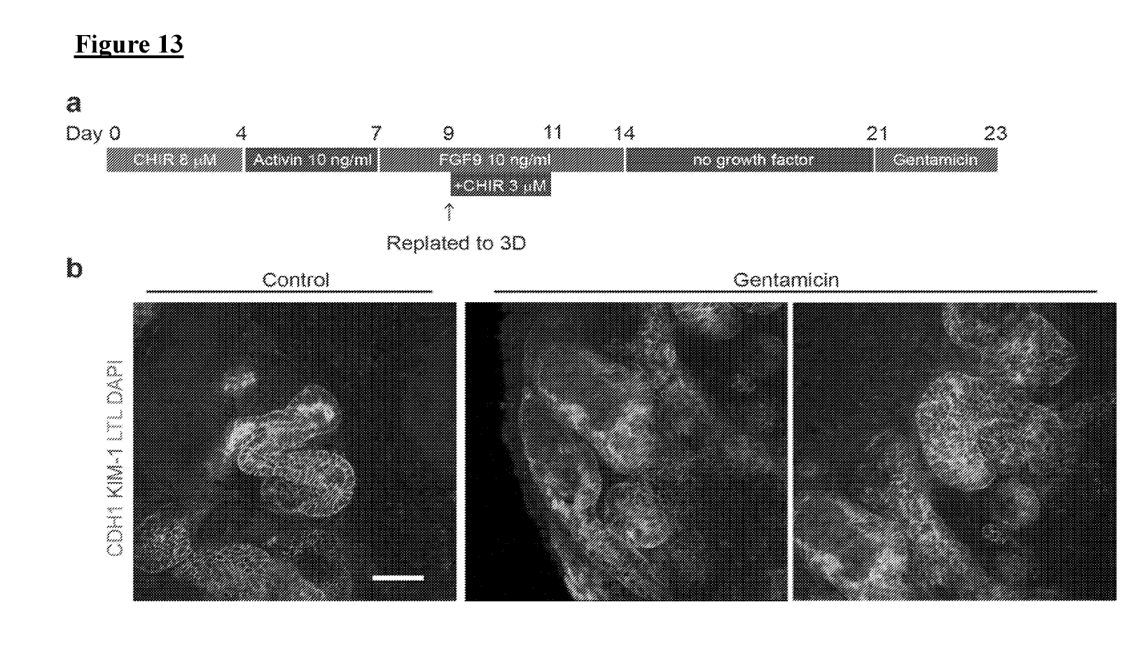

Chronic kidney disease affects 9-13% of the U.S. adult population and is a serious public health problem worldwide. Disease progression is marked by gradual, irreversible loss of nephrons, the individual functional units of the kidney. The ability to generate functional kidney tissue from hPSCs may allow the development of cell therapies for kidney disease as well as strategies for modeling kidney development and disease and for drug screening. Nephrons are made up of glomeruli, which filter the blood plasma into a multicomponent tubular system that reabsorbs and/or secretes solutes and water to produce urine. The many different epithelial cell types in nephrons have complicated efforts to generate them in vitro. However, studies have shown that all of these epithelial cell types except those in the collecting ducts derive from a multipotent nephron progenitor cell (NPC) population present in the metanephric cap mesenchyme during kidney development. The NPCs, which express the markers SIX2, SALL1, WT1, and PAX2, are found in humans only during kidney organogenesis, which ceases by birth and cannot be reinitiated after birth, even during repair after kidney injury. However, these NPC markers are also expressed in the more primitive mesonephric mesenchyme that derives from the anterior intermediate mesoderm (IM) and forms the transiently functional mesonephros. This suggests that careful attention to the early separation of anterior versus posterior intermediate mesoderm (IM) fate is likely to be critical for the proper induction of metanephric NPCs (FIG. 13a).

Several studies have attempted to differentiate mouse and human PSCs into cells of the kidney lineage. Published protocols to produce SIX2+ NPCs from hPSCs have several limitations. First, differentiation efficiency is too low for large-scale production of NPCs. One explanation for this may be that most protocols have not distinguished anterior from posterior IM in early steps of directed differentiation. The metanephric mesenchyme derives from cells of the primitive streak, which persist as cells of the posterior IM. In contrast the ureteric bud, the precursor to the adult kidney collecting duct system, originates in the anterior IM, a cell population incapable of giving rise to the metanephric mesenchyme. Second, existing protocols use poorly defined components, such as mouse embryonic spinal cord, which would not be suitable for clinical applications. Finally, previous studies could generate nephron rudiments, but not mature nephron segments containing the mature kidney epithelial cell types or a single contiguous nephron-like structure with characteristics of multiple nephron segments, precluding their use for modeling human kidney development, disease, and injury.

Here the Inventors describe an efficient, chemically defined system for differentiating hPSCs into multipotent NPCs capable of forming nephron-like structures. By carefully recapitulating the stages of metanephric kidney development in two-dimensional monolayer culture, the Inventors generate NPCs that co-express the critical markers SIX2, SALL1, WT1, and PAX2 with 90% efficiency within 9 days of initiation of differentiation—a substantial improvement over previous methods (FIG. 13b). The NPCs exhibit the developmental potential of their in vivo counterparts and can spontaneously form PAX8+LHX1+ renal vesicles that self-pattern into epithelial nephron structures. This process can be markedly enhanced by mimicking in vivo nephron induction by transiently treating the NPCs with the GSK-3β inhibitor CHIR99021 (CHIR) and FGF9 to induce renal vesicle formation. This is followed by self-organizing differentiation into continuous structures with sequential characteristics of podocytes, proximal tubules, loops of Henle, and distal tubules in both 2D and 3D culture.

### SUMMARY OF THE INVENTION

Described herein is a method for generating metanephric mesenchyme, including providing a quantity of human pluripotent stem cells (“hPSCs”), generating late primitive streak cells, inducing formation of posterior intermediate mesoderm cells, and differentiating into metanephric mesenchyme cells. In other embodiments, the human pluripotent stem cells are human embryonic stem cells (“hESCs”). In other embodiments, the human pluripotent stem cells are human induced pluripotent stem cells (“hiPSCs”). In other embodiments, generating late primitive streak cells includes culturing in CHIR99021 for about 3-5 days. In other embodiments, the method further includes addition of Noggin. In other embodiments, inducing formation of posterior intermediate mesoderm cells includes culturing in the presence of activin for about 2-4 days. In other embodiments, differentiating into metanephric mesenchyme cells includes addition of FGF9. In other embodiments, the metanephric mesenchyme lineage cells are further differentiated into nephronic progenitor cells (NPCs) by addition of CHIR99021.

In other embodiments, late primitive streak cells express one or more of: T and TBX. In other embodiments, posterior intermediate mesoderm cells express one or more of: WT1 and HOXD11. In other embodiments, metanephric mesenchyme lineages cells express one or more of: SIX2, SALL1, WT1, and PAX2. In other embodiments, NPCs express one or more of: SIX2, SALL1, WT1, PAX2, and EYA1. In other embodiments, differentiation into metanephric mesenchyme cells is at least 50% efficient. In other embodiments, differentiation into metanephric mesenchyme cells is at least 70% efficient.

Further described herein is a composition of metanephric mesenchyme cells generated by a method for generating metanephric mesenchyme, including providing a quantity of human pluripotent stem cells (“hPSCs”), generating late primitive streak cells, inducing formation of posterior intermediate mesoderm cells, and differentiating into metanephric mesenchyme cells. In other embodiments, the human pluripotent stem cells are human embryonic stem cells (“hESCs”). In other embodiments, the human pluripotent stem cells are human induced pluripotent stem cells (“hiPSCs”). In other embodiments, generating late primitive streak cells includes culturing in CHIR99021 for about 3-5 days. In other embodiments, the method further includes addition of Noggin. In other embodiments, inducing formation of posterior intermediate mesoderm cells includes culturing in the presence of activin for about 2-4 days. In other embodiments, differentiating into metanephric mesenchyme cells includes addition of FGF9. In other embodiments, the metanephric mesenchyme lineage cells are further differentiated into nephronic progenitor cells (NPCs) by addition of CHIR99021. In other embodiments, late primitive streak cells express one or more of: T and TBX. In other embodiments, posterior intermediate mesoderm cells express one or more of: WT1 and HOXD11. In other embodiments, metanephric mesenchyme lineages cells express one or more of: SIX2, SALL1, WT1, and PAX2. In other embodiments, NPCs express one or more of: SIX2, SALL1, WT1, PAX2, and EYA1. In other embodiments, differentiation into metanephric mesenchyme cells is at least 50% efficient. In other embodiments, differentiation into metanephric mesenchyme cells is at least 70% efficient. A composition of nephron progenitor cells generated by the described method.

Described herein is a method of generating kidney organoids, including providing a quantity of nephron progenitor cells (“NPCs”), and culturing the NPCs in a suspension culture for at about 11 days. In other embodiments, the method includes addition of one or more of: CHIR99021 and FGF9. In other embodiments, the kidney organoids comprise one or more cell types selected from: podocyte-like cells, proximal tubules, descending limbs of Henle, thick ascending limbs of Hendle, and distal convoluted tubules. In other embodiments, podocyte-like cells express one or more of: NPHS1+, PODXL+, and WT1+. In other embodiments, proximal tubules express one or more of: LTL+ and AQP1+. In other embodiments, descending limbs of Henle express one or more of: CDH1+ and AQP1+. In other embodiments, thick ascending limbs of Henle express one or more of CDH1+ and UMOD+. In other embodiments, distal convoluted tubules express one or more of CDH1+UMOD−. In other embodiments, NPCs are derived from human pluripotent stem cells (“hPSCs”). In other embodiments, hPSCs are derived from a patient suffering a disease mutation. In other embodiments, hPSCs have been genomically edited using CRISPR.

Also described herein is a quantity of organoids made by a method of generating kidney organoids, including providing a quantity of nephron progenitor cells (“NPCs”), and culturing the NPCs in a suspension culture for at about 11 days. In other embodiments, the method includes addition of one or more of: CHIR99021 and FGF9. In other embodiments, the kidney organoids comprise one or more cell types selected from: podocyte-like cells, proximal tubules, descending limbs of Henle, thick ascending limbs of Hendle, and distal convoluted tubules. In other embodiments, podocyte-like cells express one or more of: NPHS1+, PODXL+, and WT1+. In other embodiments, proximal tubules express one or more of: LTL+ and AQP1+. In other embodiments, descending limbs of Henle express one or more of: CDH1+ and AQP1+. In other embodiments, thick ascending limbs of Henle express one or more of CDH1+ and UMOD+. In other embodiments, distal convoluted tubules express one or more of CDH1+UMOD−. In other embodiments, NPCs are derived from human pluripotent stem cells (“hPSCs”). In other embodiments, hPSCs are derived from a patient suffering a disease mutation. In other embodiments, hPSCs have been genomically edited using CRISPR.

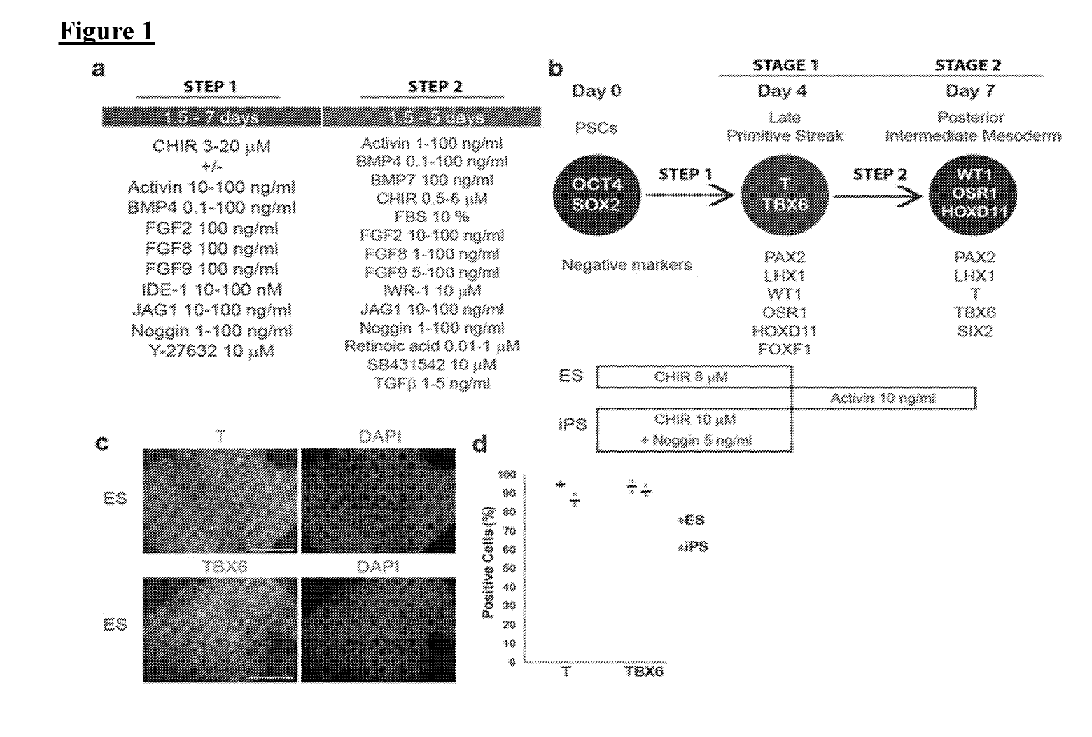

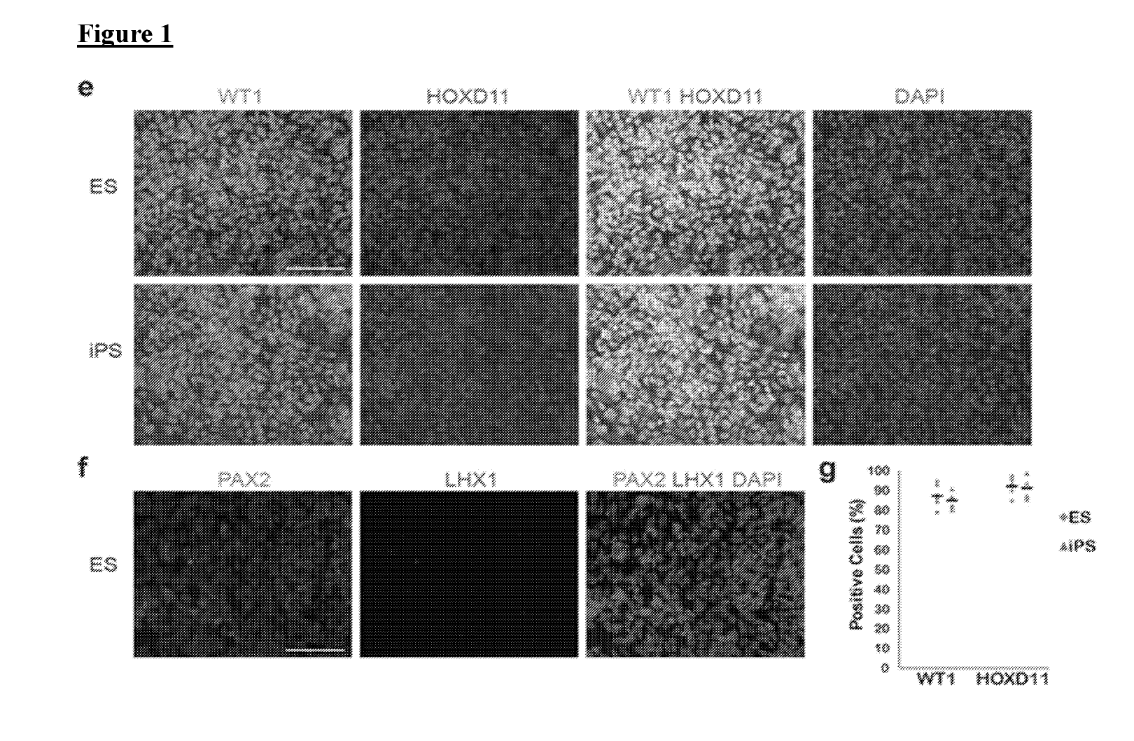

FIG. 1. Differentiation of hPSCs into posterior intermediate mesoderm. (a) Agents that were tested for the induction of late primitive streak and posterior IM. (b) Diagram and the protocol of differentiation of hPSCs sequentially into late primitive streak and posterior intermediate mesoderm (IM) with markers identifying both states by their presence or absence. In the protocol for posterior IM hESCs and hiPSCs were differentiated with CHIR 8 μM and 10 μM respectively. For hiPSCs Noggin 5 ng/ml was also required. (c) Immunocytochemistry for T and TBX6 in hESCs on day 4 of the differentiation with CHIR 8 μM. (d) Percentage of T or TBX6 positive cells in hESCs and hiPSCs on day 4. Black bars indicate mean values. n=3 for hESCs; n=3 for hiPSCs. (e) Immunocytochemistry for WT1 and HOXD11, posterior IM markers, in hESCs and hiPSCs on day 7. (f) Immunocytochemistry for PAX2 and LHX1, anterior IM markers in hESCs on day 7. (g) Percentage of WT1 or HOXD11 positive cells in hESCs and hiPSCs on day 7. Black bars indicate mean values. n=7 for hESCs; n=4 for hiPSCs. Scale bars: 100 μm.

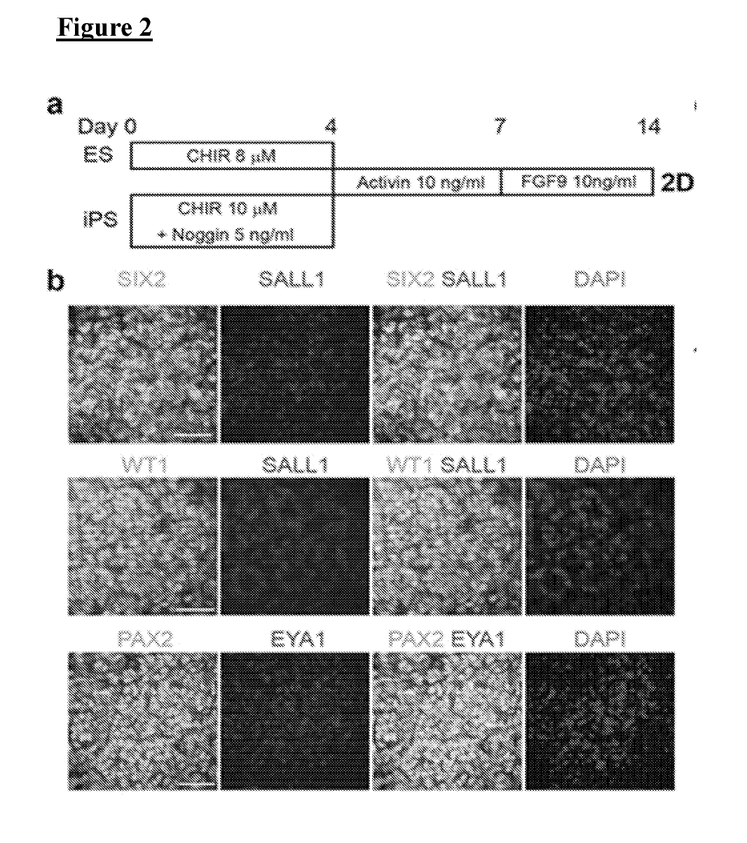

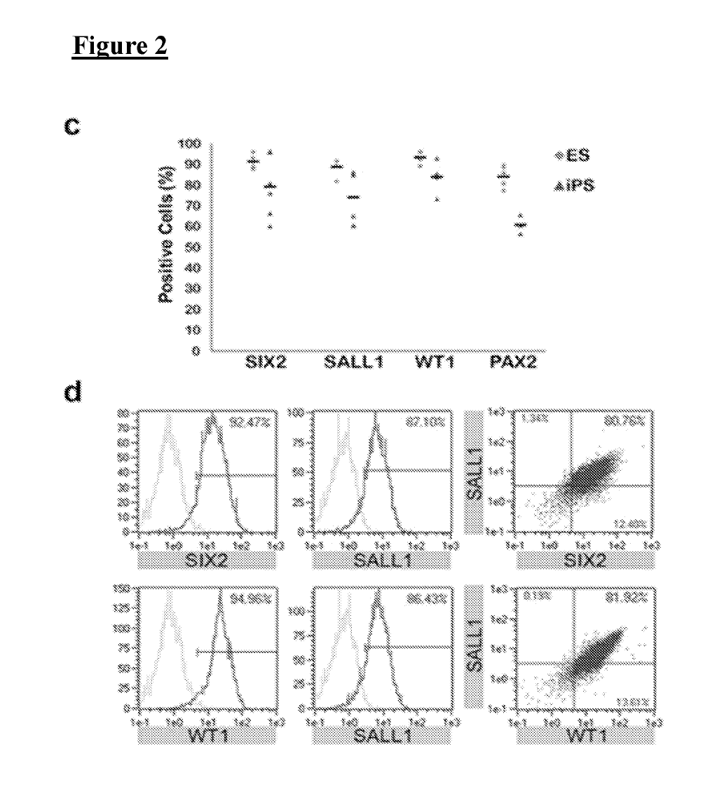

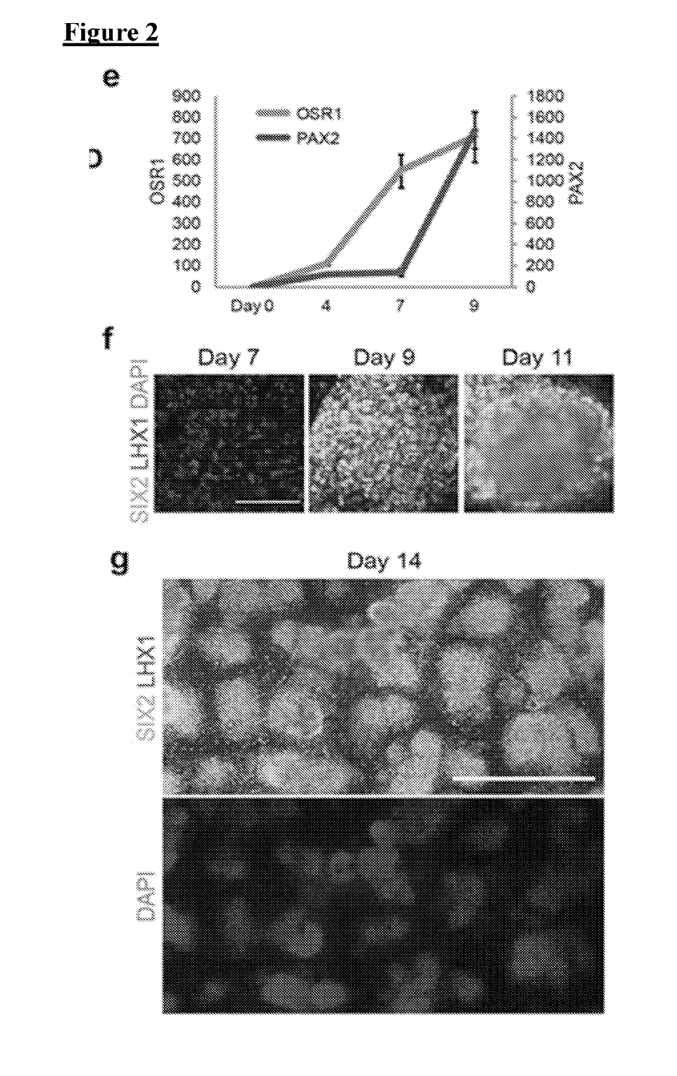

FIG. 2. Differentiation into nephron progenitor cells and spontaneous formation of renal vesicles. (a) The protocol for the induction of nephron progenitor cells. (b) Immunocytochemistry for SIX2, SALL1, WT1, PAX2 and EYA1, markers of nephron progenitor cells, on day 9 in cells differentiated from hPSCs using protocol depicted in (a). Scale bars: 50 (c) Percentage of cells positive for SIX2, SALL1, WT1 or PAX2 in hESCs and hiPSCs on day 9. n=7, 5, 4 or 7 for SIX2, SALL1, WT1 or PAX2 respectively in hESCs. n=6, 4, 4, or 3 for SIX2, SALL1, WT1 or PAX2 respectively in hiPSCs. Black bars indicate mean values. (d) Flow cytometry for SIX2, SALL1 and WT1 in hESCs on day 8. Samples stained with secondary antibodies alone were used as controls (gray). (e) Time course of gene expression of OSR1 and PAX2 in hESCs from day 0 to 9. OSR1 was upregulated on days 7 and 9 while PAX2 was upregulated on day 9. n=2. Data represent mean+/−SEM. (f, g) Time course of SIX2 and LHX1 expression from day 7 to 14 in hESCs treated with FGF9 10 ng/ml. (g) When FGF9 was continued to day 14, SIX2 expression was sustained, but spontaneous induction of LHX1+ cells was consistent with maturation to renal vesicles. The images are representative of the density of renal vesicle formation in the culture dishes. Scale bars: 100 μm (f), 1 mm (g).

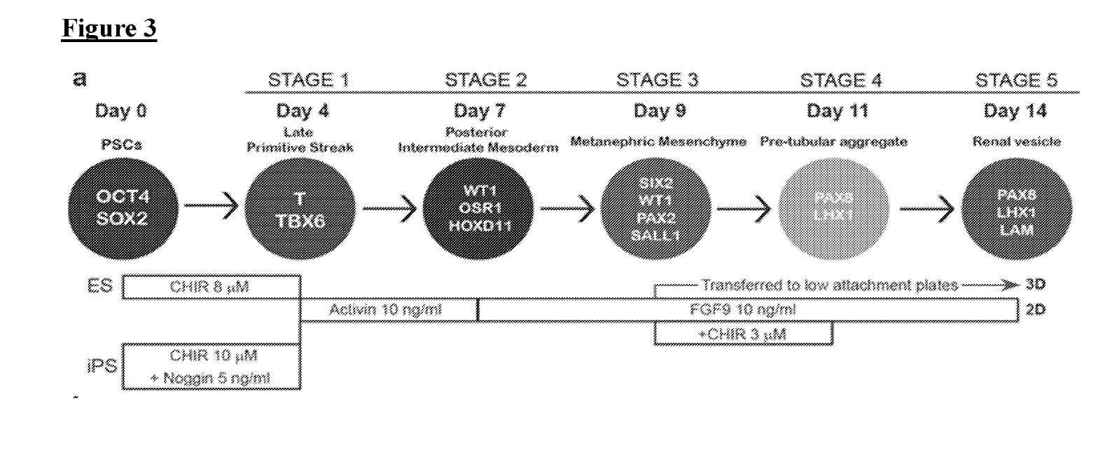

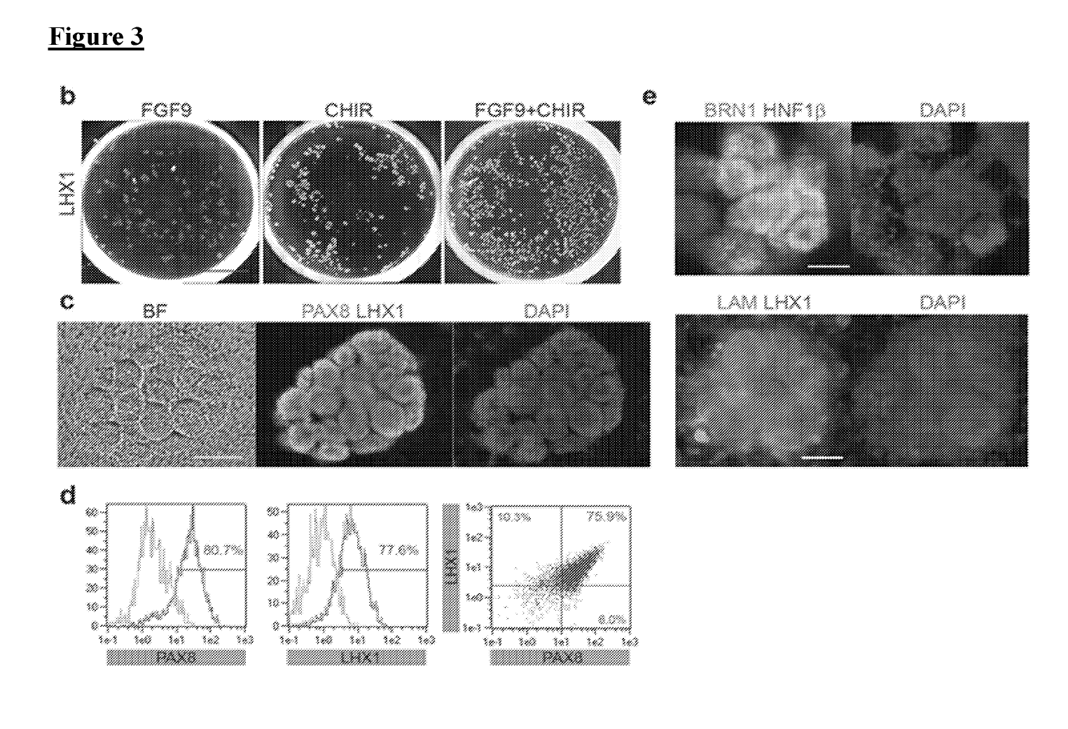

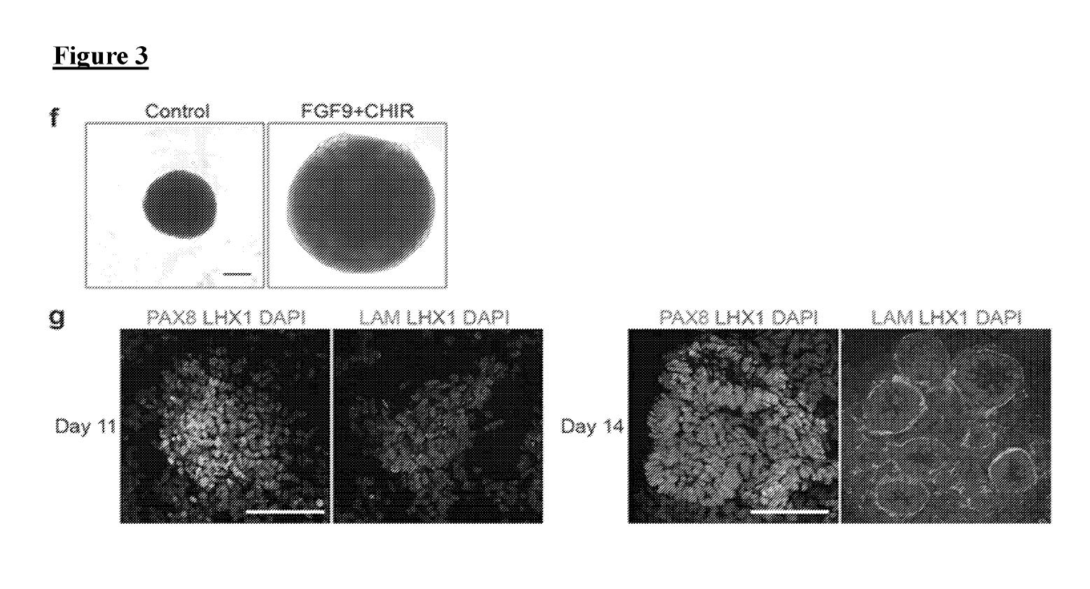

FIG. 3. Induction of pre-tubular aggregates and renal vesicles from nephron progenitor cells. (a) Diagram of differentiation into renal vesicles. (b) Whole-well scan for LHX1 in 24-well on day 14 of differentiation. The combination of FGF9 10 ng/ml and transient CHIR 3 μM treatment enhanced LHX1 expression. n=2. Scale bar: 5 mm. (c) Representative images of brightfield and immunocytochemistry for PAX8 and LHX1 in hESCs on day 14. Scale bar: 100 μm. (d) Flow cytometry for PAX8 and LHX1 in hESCs on day 14. Samples treated with secondary antibodies alone were used as controls (gray). (e) Immunocytochemistry for BRN1, HNF1β and LAM (Laminin) on day 14 of differentiation. n=6. 50 μm (the lower panel). (f) Brightfield imaging of the organoids that formed in culture after cells were resuspended on day 9, transferred to ultra-low attachment 96-well plates and studied on day 14. Controls were cultured in the basic differentiation medium (ARPMI) after resuspension. FGF9 and CHIR increased the size of the organoids. Scale bar: 100 μm. (g) Whole-mount staining of the organoids on day 11 and 14. Polarized structures surrounded by Laminin were found on day 14, suggesting the differentiation into renal vesicles. n=2. Scale bars: 100 μm.

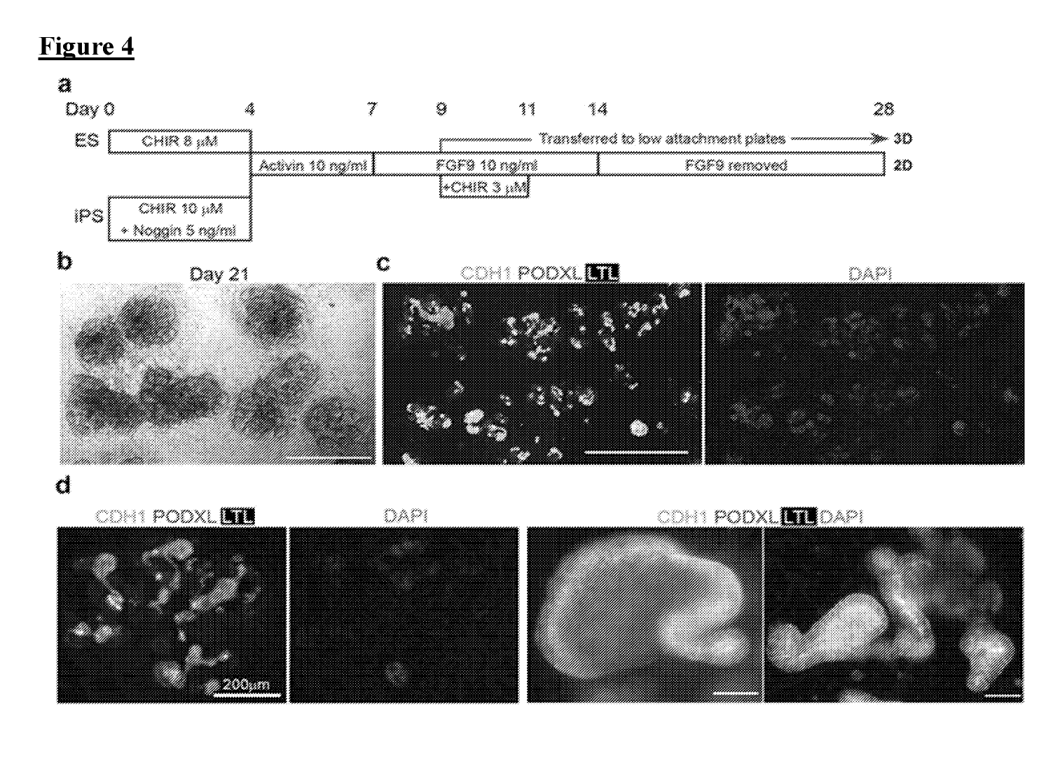

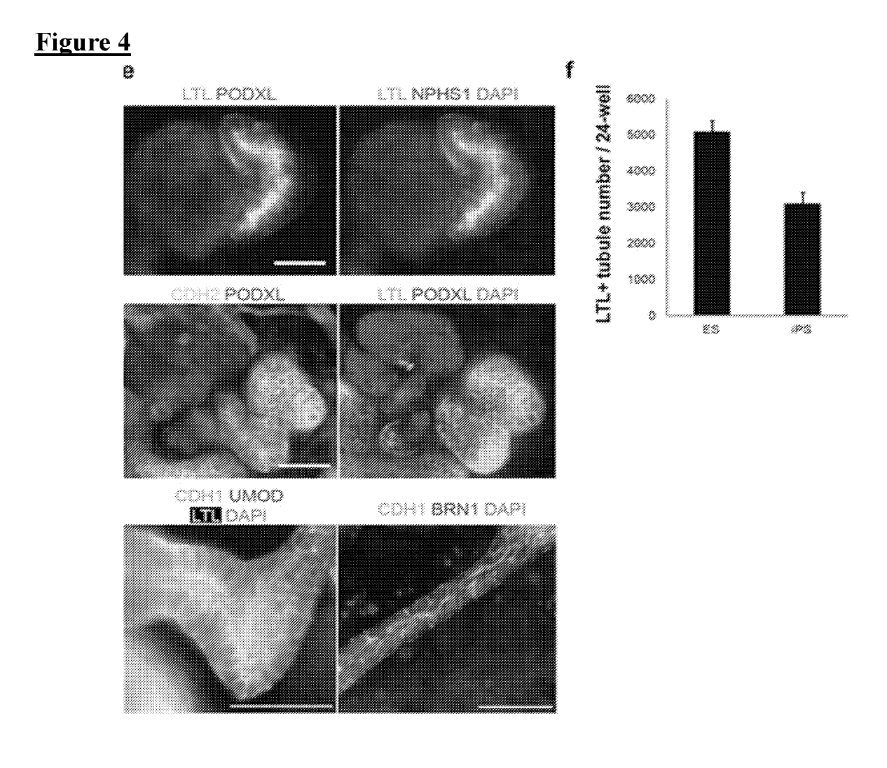

FIG. 4. Self-organizing nephron formation in 2D culture. (a) The protocol for the induction of nephrons. (b) Representative brightfield images on day 21 of differentiation. Scale bar: 1 mm. (c) Immunocytochemistry for CDH1, PODXL and LTL on day 21. Scale bar: 1 mm. (d, e) Immunocytochemistry for podocyte (PODXL, NPHS1) proximal tubule (CDH2, LTL), loop of Henle (CDH1, UMOD) and distal tubule (CDH1, BRN1) markers on days 21-28. n=7. Scale bars: 50 μm unless otherwise indicated. (f) The number of LTL+ tubules in structures derived from hESCs and hiPSCs on day 21; n=2. Values were calculated from 10 fields (1 mm²/field) in each sample. Data represent mean+/−SEM. CDH1: Cadherin-1 (E-cadherin). PODXL: Podocalyxin-like (Podocalyxin). LTL: lotus tetragonolobus lectin. NPHS1: Nephrin. UMOD: Uromodulin. CDH2: Cadherin-2 (N-cadherin).

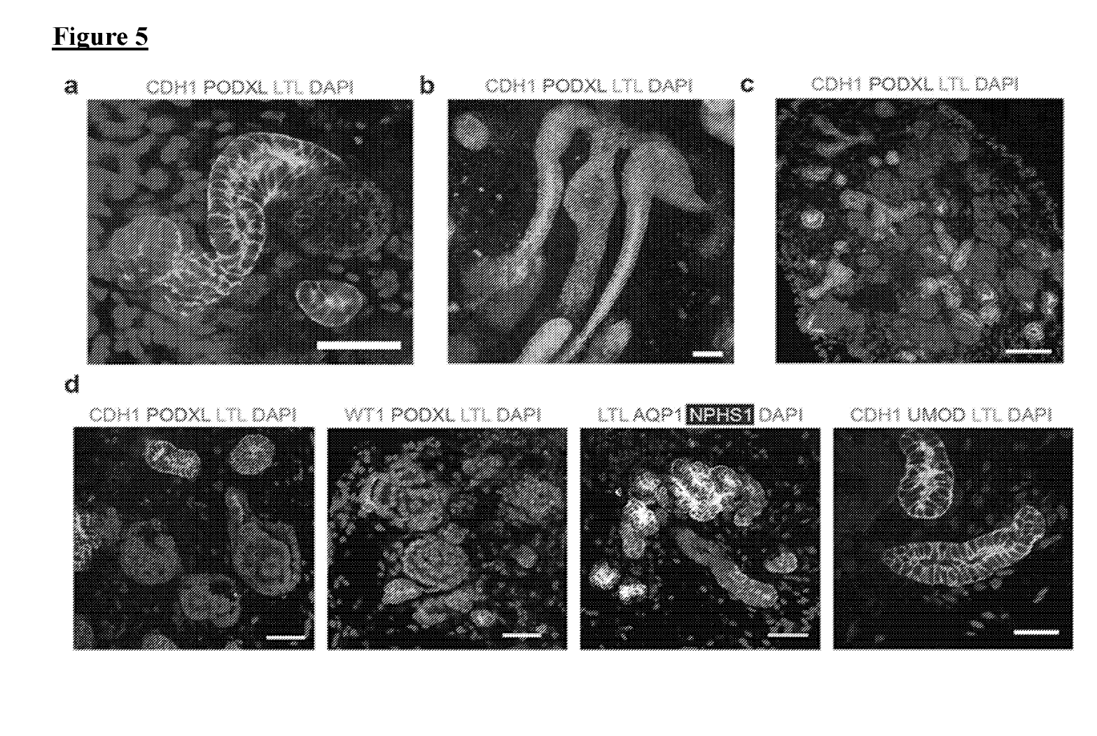

FIG. 5. Self-organizing nephron formation in 3D culture. (a, b) Whole-mount staining for CDH1, PODXL and LTL on day 28 (a) and 35 (b) using protocol in FIG. 4a. Scale bar: 50 μm (c, d) Representative immunohistochemistry in structures derived from hESCs and hiPSCs on day 21-28. n=5. Scale bars: 50 μm. CDH1: Cadherin-1 (E-cadherin). PODXL: Podocalyxin-like (Podocalyxin). LTL: lotus tetragonolobus lectin. AQP1: aquaporinl. NPHS1: Nephrin. UMOD: Uromodulin. (c) Low magnification. (d) High magnification. (e) Representative electron microscopy images of glomerulus-like and tubule regions of kidney organoids derived from hESCs. Middle panels represent higher magnification enlargement of the square-enclosed regions within left panels. n=5. Samples were taken at 21 days with the exception of the top right panel which was taken at day 18 and did not have transient CHIR treatment. Dotted lines: Bowman's capsule. Arrows: foot process. Allow heads: tight-junction. Asterisks: mitochondria. Hashes: brush border-like structures. (f) Electron microscopy images of normal human kidneys showing foot processes (upper panel) and brush borders (lower panel).

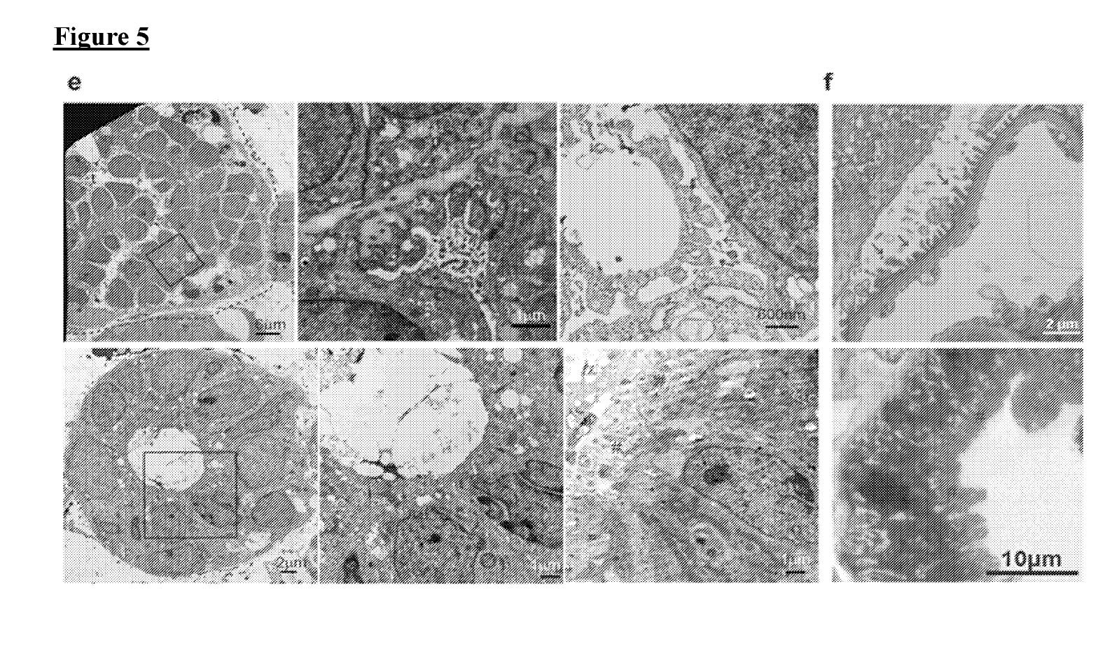

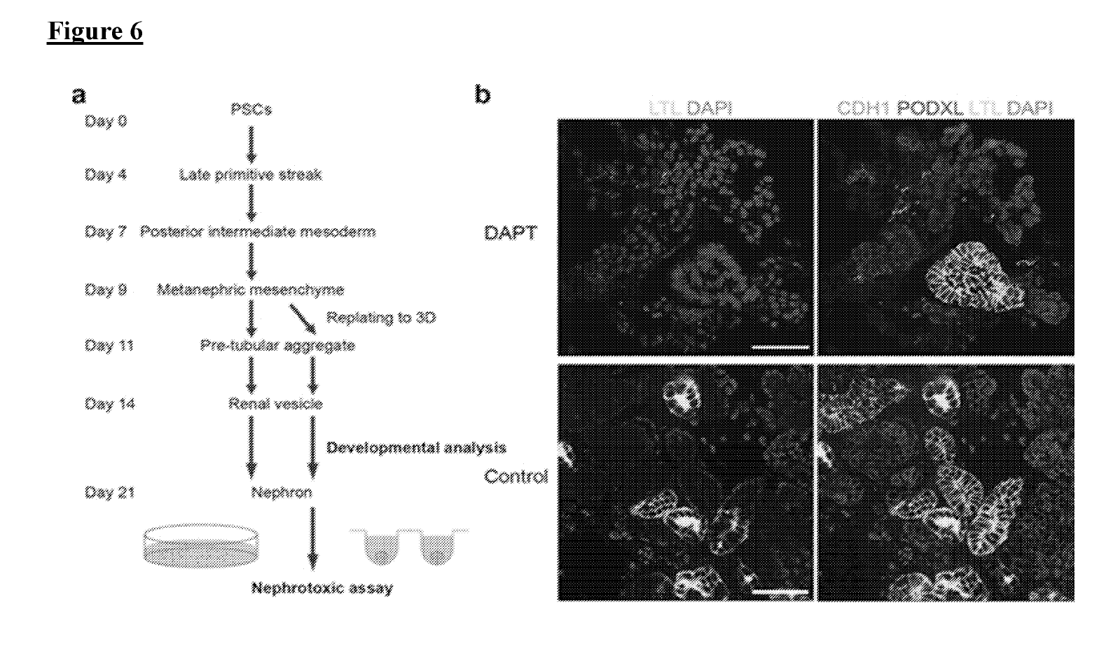

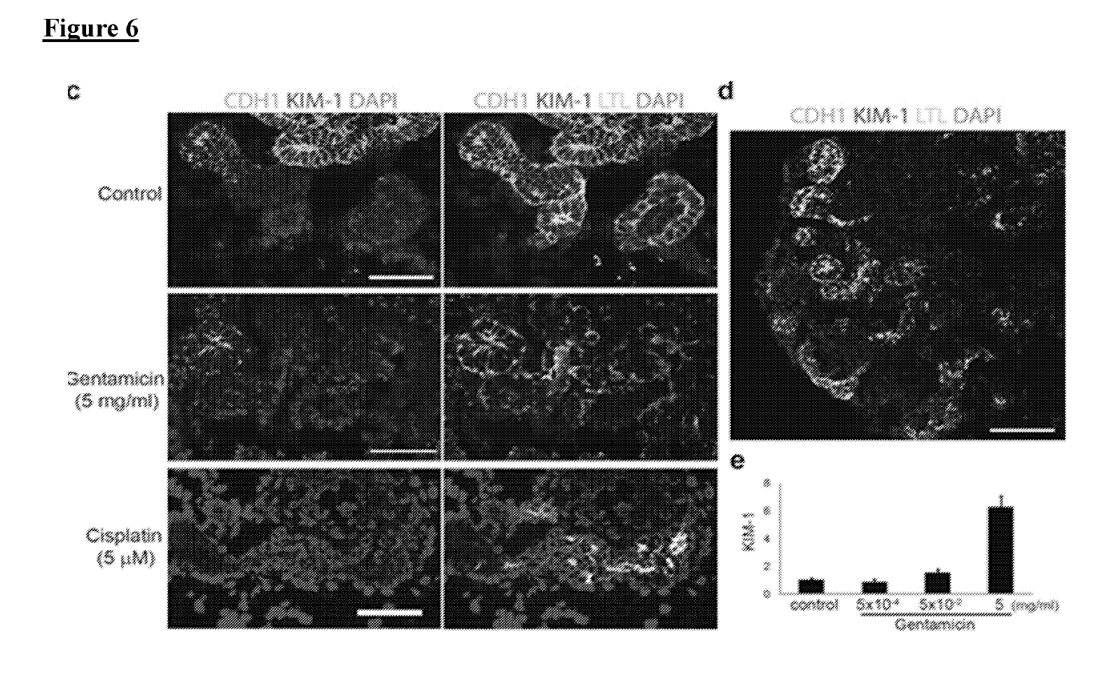

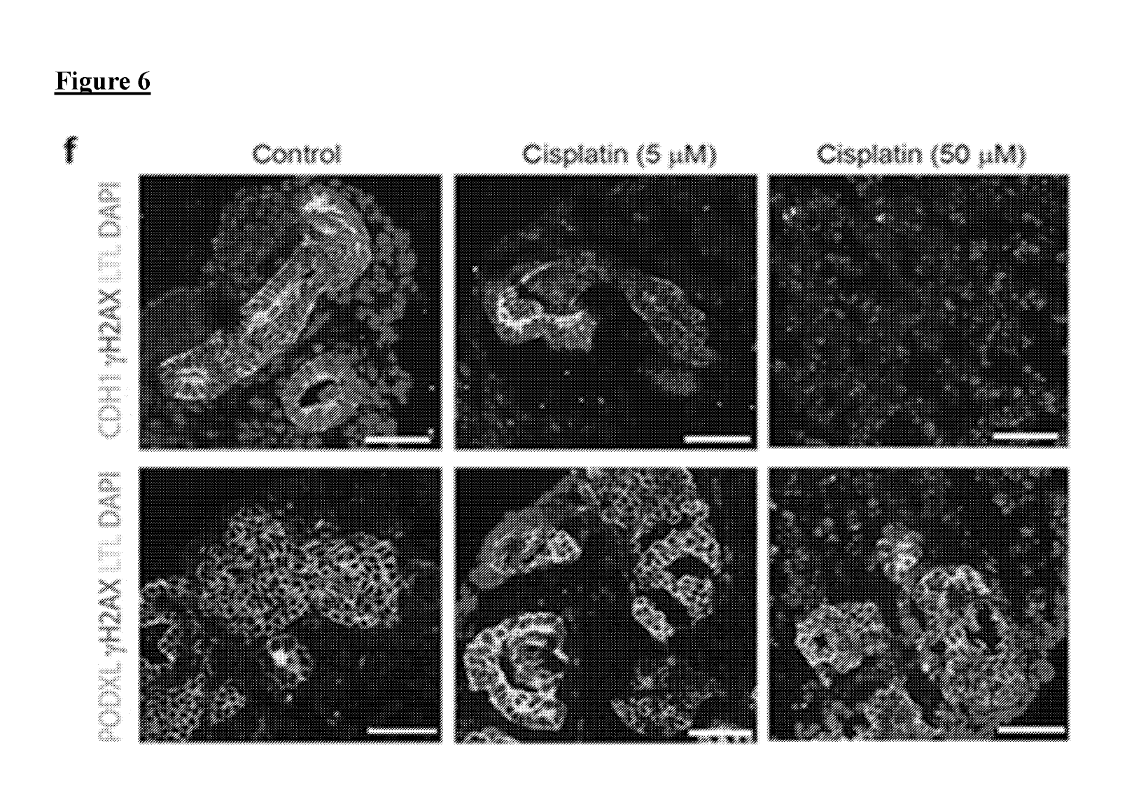

FIG. 6. Modeling kidney development and injury in kidney organoids. (a) Schematic for kidney development analysis and nephrotoxicity assay. (b) Representative images of immunohistochemistry in structures derived from hESCs treated with DAPT 10 μM from day 14 to 21. n=4. Notch inhibition suppressed proximal tubule formation. Scale bars: 50 μm. (c) Representative immunohistochemistry in structures treated with gentamicin (5 mg/ml) from day 21 to 23 or cisplatin (5 μM) from day 21 to 22. n=6. Scale bars: 50 μm. Gentamicin and cisplatin induced the upregulation of KIM-1, and cisplatin suppressed CDH1 expression. (d) A low magnification image of gentamicin-treated organoids. Scale bar: 100 μm. CDH1: Cadherin-1 (E-cadherin). PODXL: Podocalyxin-like (Podocalyxin). LTL: lotus tetragonolobus lectin. KIM-1: kidney injury molecule-1. (e) Real-time quantitative PCR of KIM-1 in kidney organoids treated with gentamicin at indicated doses. Data is expressed as mean+/−SEM (n=10). (f) Representative immunohistochemistry of organoids treated with cisplatin (5 or 50 μM) from day 23 to 24 (24 hours). n=4. Scale bars: 50 μm.

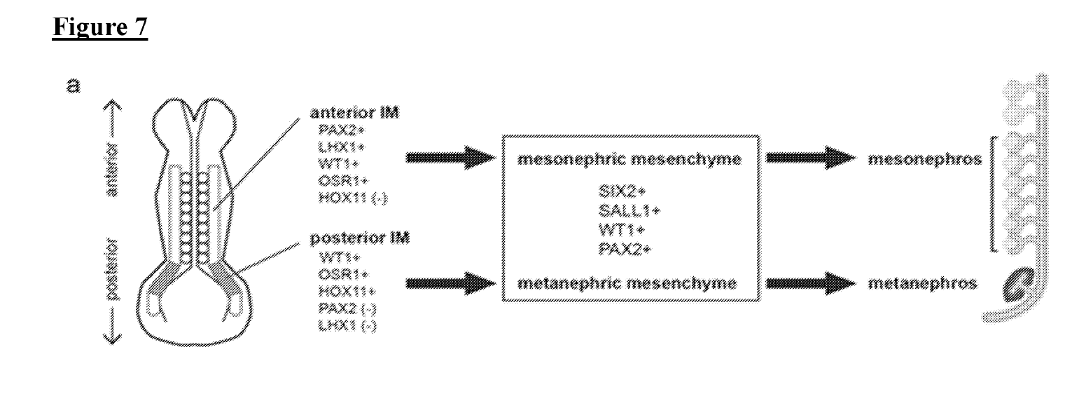

FIG. 7. Metanephric development and published protocols. (a) A schematic illustration of intermediate mesoderm and subsequent differentiation into mesonephros and metanephros. (b) The summary and comparison of published protocols and the Inventors' new protocol. Takasato et al. Nat Cell Biol. 2014. Taguchi et al. Cell Stem Cell. 2014. RA: retinoic acid.

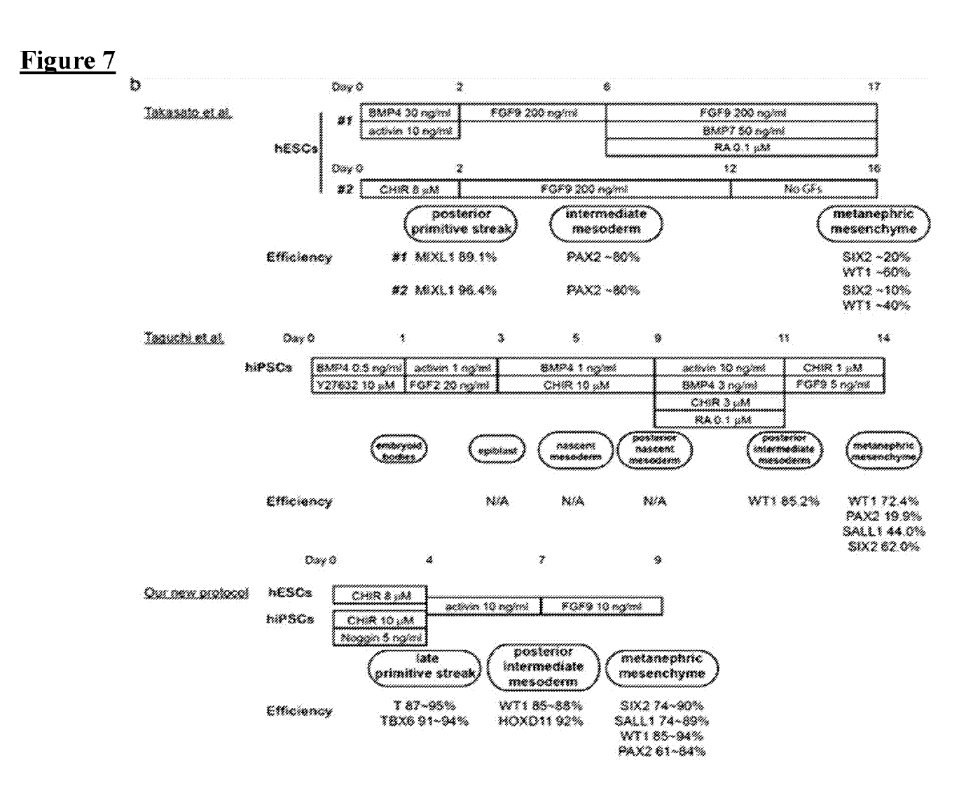

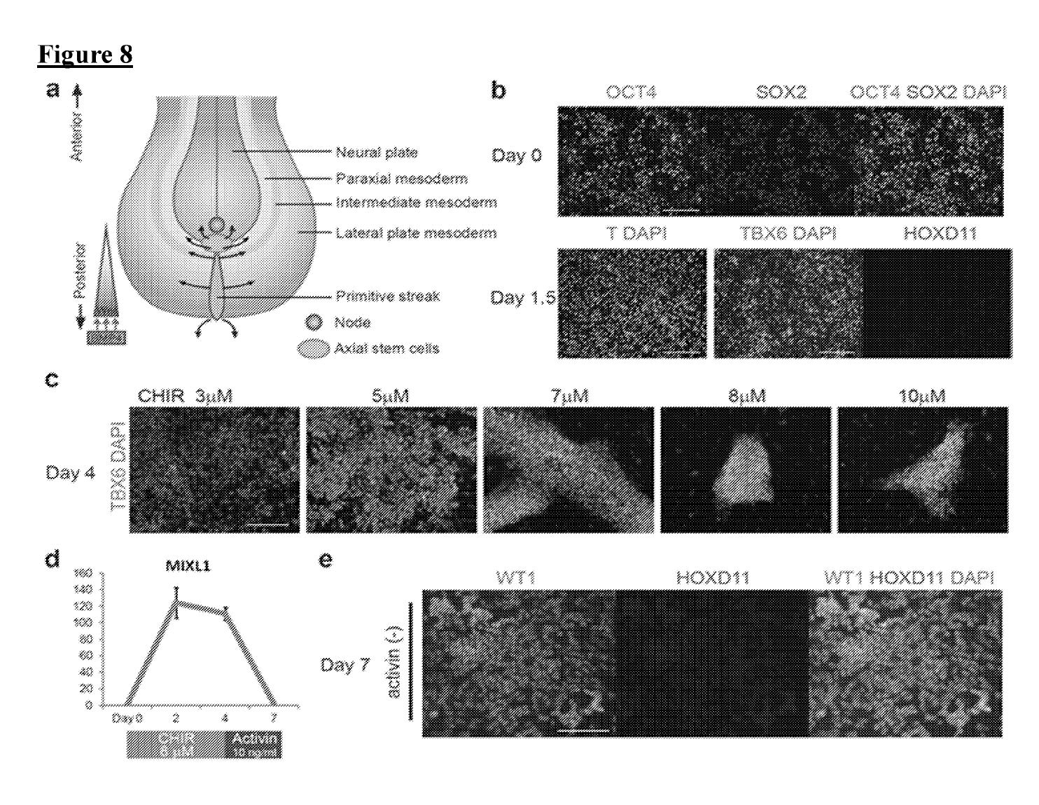

FIG. 8. Adjustment of the dose and CHIR treatment time. (a) A schematic illustration of primitive streak and subsequent differentiation into each mesoderm lineage. (b) Pluripotency was evaluated by staining with OCT4 and SOX2 before the differentiation. hESCs differentiated with CHIR 5 μM were positive for T and TBX6 on day 1.5 of differentiation, but cells did not stain for HOXD11. Scale bars: 200 μm. (c) Immunocytochemistry on day 4 of the differentiation with CHIR (3 to 10 Sustained TBX6 expression was observed when the cells were differentiated with high doses of CHIR (7-10 μM) Scale bar: 100 μm. (d) Real-time PCR for MIXL1 in hESCs from day 0 to 7. hESCs were differentiated with CHIR 8 μM for 4 days, and activin 10 ng/ml for 3 days. MIXL1, another marker for primitive streak also showed sustained expression at least until day 4 of the differentiation. Expression returned to very low levels by day 7. n=2. (e) Staining with WT1 and HOXD11 on day 7 of the differentiation. hESCs were differentiated with CHIR 8 μM for 4 days and subsequently with the basic medium (ARPMI) for 3 days. Cells expressed WT1, but not HOXD11. Scale bar 100 μm.

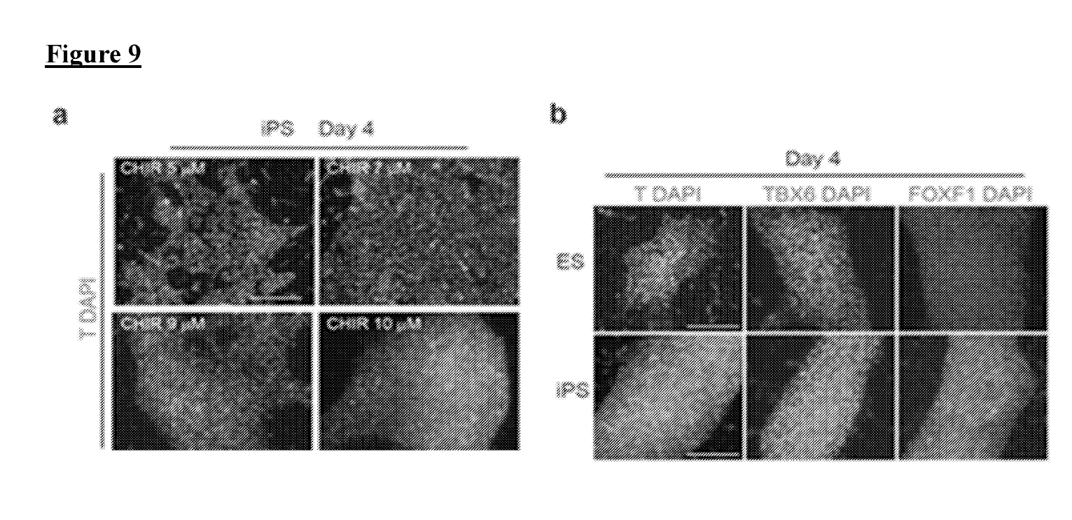

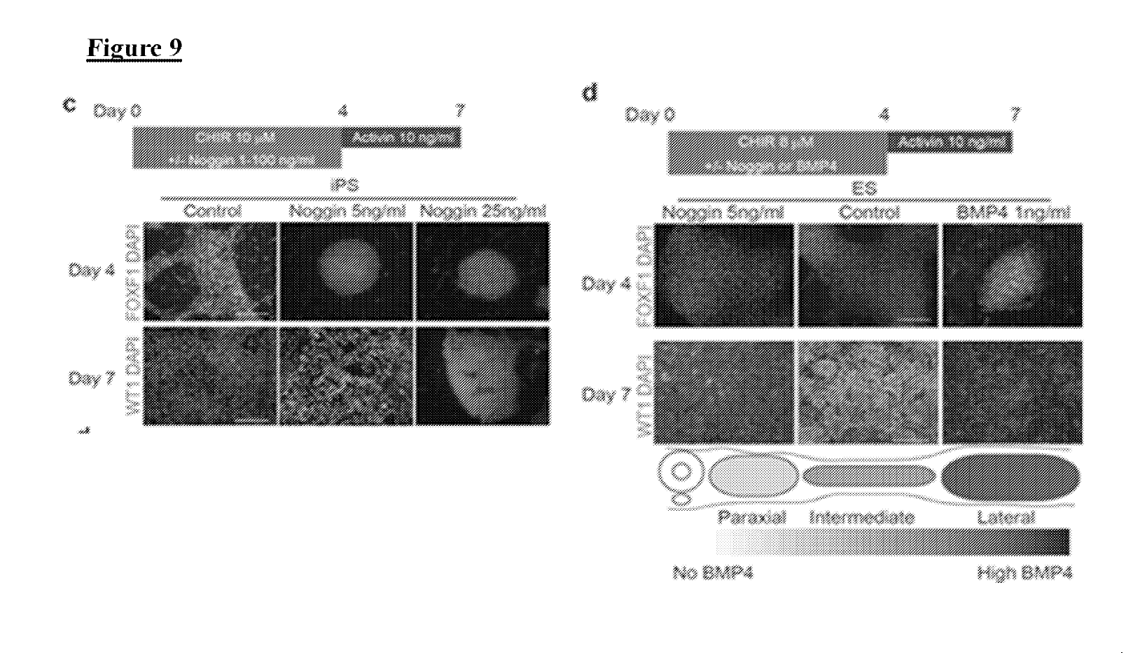

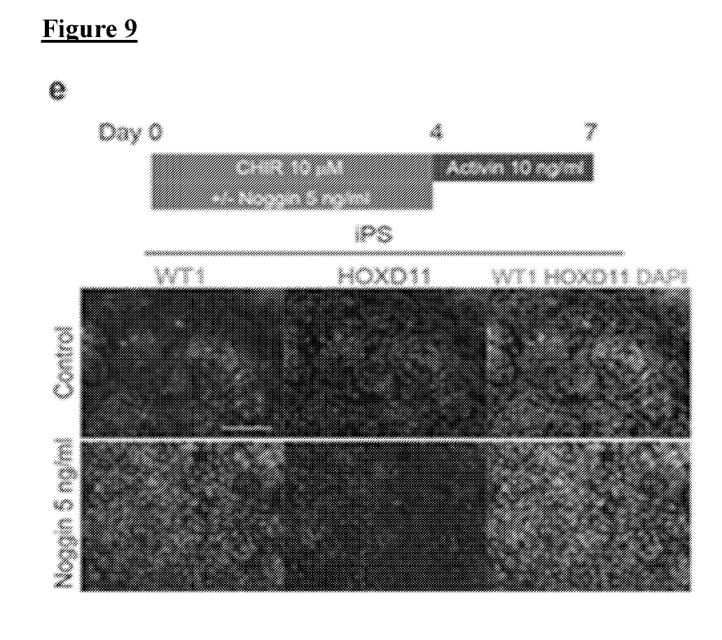

FIG. 9. Protocol adjustment in hiPSCs. (a) Comparison of CHIR dose in T expression on day 4. A slightly higher dose of CHIR was required for sustained T expression in hiPSCs on day 4. (b) Immunocytochemistry for T, TBX6 or FOXF1 on day 4 of differentiation. hESCs were differentiated with CHIR 8 μM, and hiPSCs were differentiated with CHIR 10 μM. Notably, FOXF1 was negative in hESCs, but was positive in hiPSCs. (c) The tested protocol and representative immunocytochemistry in hiPSCs. Noggin at >5 ng/ml suppressed FOXF1 expression on day 4. To induce WT1 expression on day 7, Noggin 5 ng/ml was optimal. (d) The differentiation protocol and staining for FOXF1 on day 4, and for WT1 on day 7 in hESCs. Either additional Noggin or BMP4 significantly suppressed WT1 expression on day 7, and BMP4 induced FOXF1 expression on day 4, suggesting endogenous BMP4 signal is optimal in hESCs with CHIR treatment alone. (e) The protocol and staining with WT1 and HOXD11 on day 7 in hiPSCs. CHIR 10 μM+Noggin 5 ng/ml followed by activin 10 ng/ml showed the most efficient differentiation into WT1+HOXD11+ cells in hiPSCs. Scale bars: 100 μm.

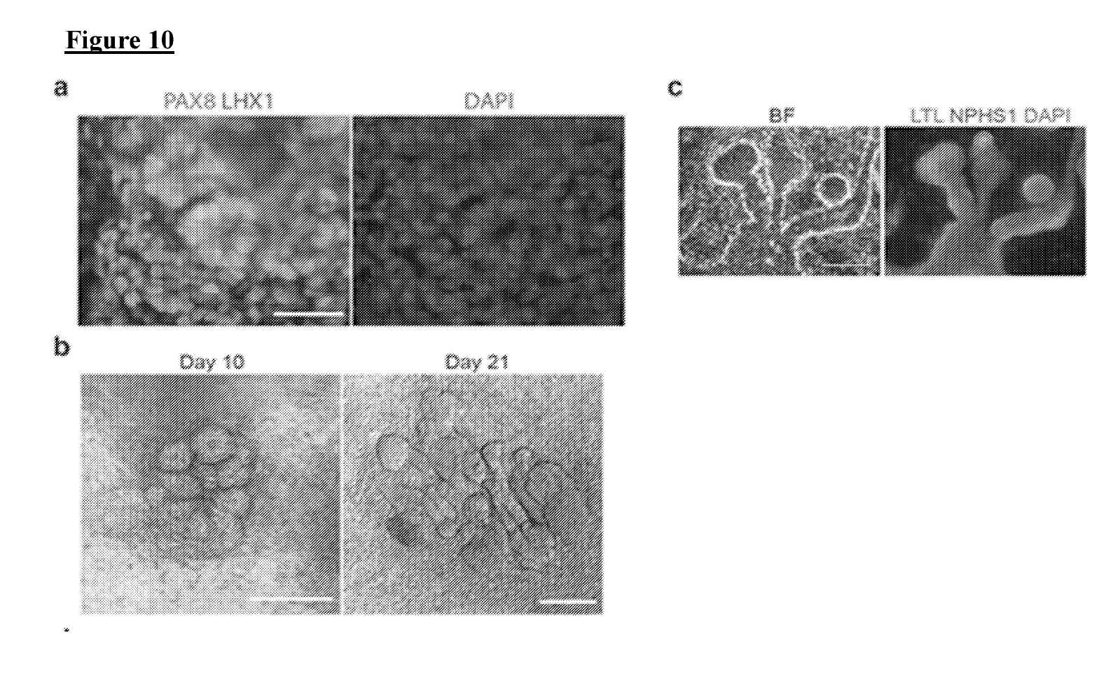

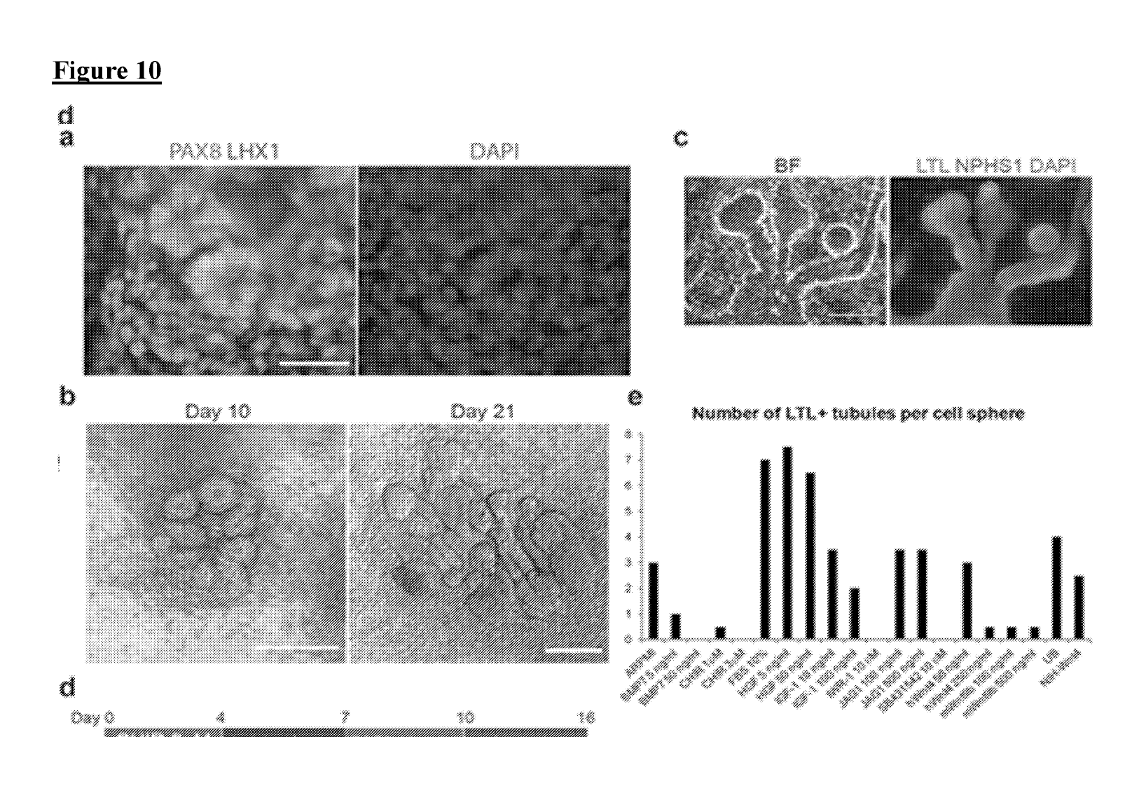

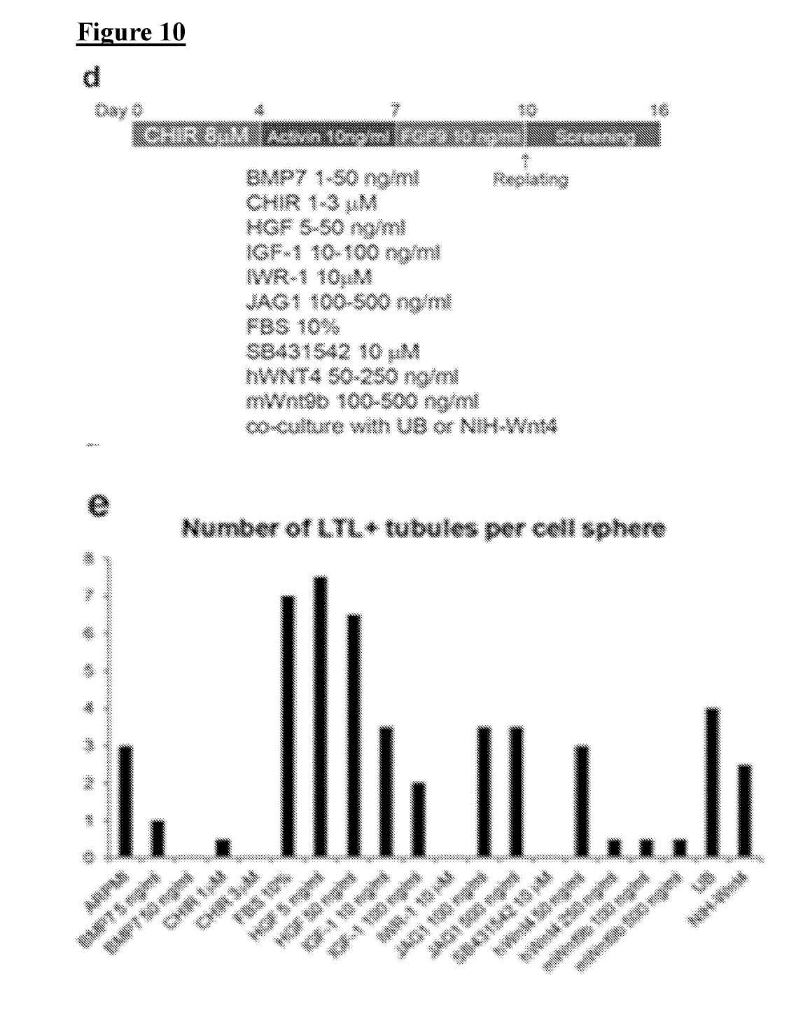

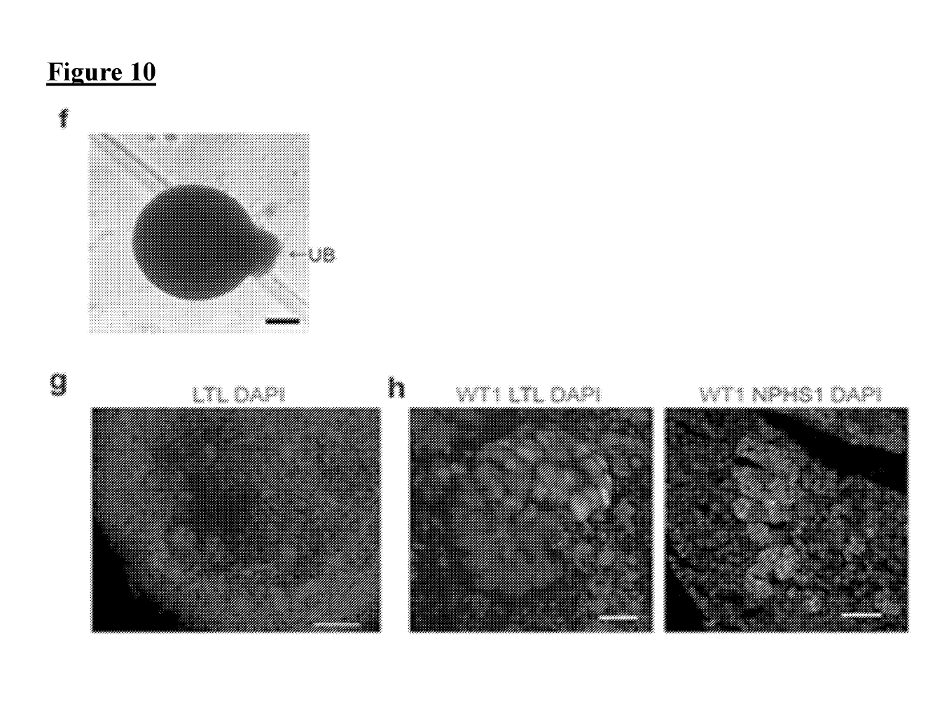

FIG. 10. Spontaneous differentiation of SIX2+ cells into nephrons and growth factor screening in 3D culture. (a) Staining with PAX8 and LHX1 in hESCs on day 10 of the differentiation. hESCs were differentiated with CHIR 8 □M for 4 days, activn 10 ng/ml for 3 days, and FGF9 10 ng/ml for 3 days. Sporadic expression of LHX1 was observed in PAX8+ cells. Scale bar: 50 μm. (b) Brighfield imaging for hESCs on day 10 and 21 of the differentiation. FGF9 was withdrawn on day 10, and cells were cultured in the basic medium by day 21. Scale bars: 50 μm. (c) Brightfield and immunocytochemistry for LTL and NPHS1 in hESCs on day 28 of the differentiation. Scale bar: 50 μm. (d) The protocol for growth factor screening. Cells were replated to 3D culture on day 10, and the listed growth factors and small molecules were tested. (e) The number of LTL+ tubules in the organoids. HGF showed a tendency to increase LTL+ tubules. n=2. (f) Brightfield imaging of 3D co-culture with an ureteric bud cell sphere. Scale bar: 100 μm. (g) Whole-mountstaining for LTL in the organoids on day 16. Scale bar: 100 μm. (h) Immunohistochemistry of the organoids on day 16. Scale bars: 50 μm. LTL: lotus tetragonolobus lectin. NPHS1: Nephrin.

FIG. 11. Screening for growth factors and small molecules to induce renal vesicles. (a, b) The tested protocols for renal vesicle induction. (c) Immunocytochemistry for SIX2 and LHX1 in structures on day 14 of differentiation. Transient treatment with CHIR 3 μM from day 9 to 11, in combination with FGF9 10 ng/ml from day 7 to 14, increased the number of LHX1+ cells and suppressed SIX2 expression, suggesting mesenchymal epithelial transition. Scale bars: 50 μm. REGM: renal cell growth medium (Lonza, #CC-3190).

FIG. 12. Kidney development analysis. (a) DAPT was used to suppress Notch signaling from day 14 to 21. (b) CDH1, PODXL and LTL expression on day 21 in cells derived from hESCs in 2D culture. Scale bars: 50 (c) Percentage of LTL+ nephron structures in control and DAPT-treated on day 21. The nephron number was counted as CDH1+ tubules from 10 fields (×20 magnification) of each sample (n=2). CDH1: Cadherin-1 (E-cadherin). PODXL: Podocalyxin-like (Podocalyxin).

FIG. 13. Nephrotoxic assay. (A) The protocol for the nephrotoxic assay. Gentamicin 5 mg/ml was added from day 21 to 23. (B) Whole-mount staining on day 23 for CDH1, KIM-1 and LTL in kidney organoids derived from hESCs. Scale bars: 50 μm. CDH1: cadherin-1 (E-cadherin). PODXL: podocalyxin-like (Podocalyxin). LTL: lotus tetragonolobus lectin. KIM-1: kidney injury molecule-1.

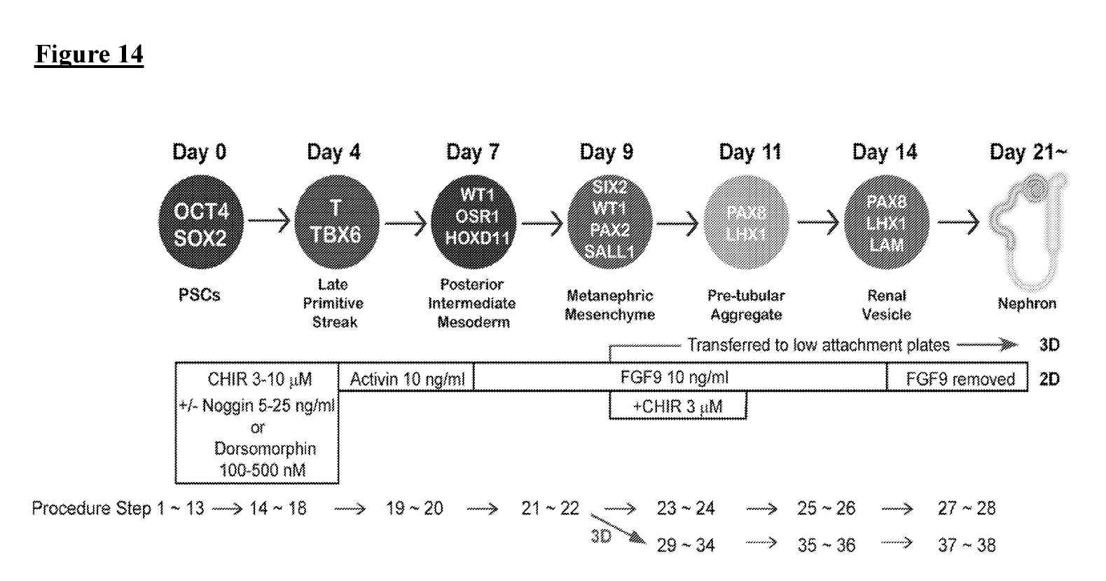

FIG. 14. The differentiation protocols into kidney organoids from hPSCs. The diagram shows markers for each step of differentiation in a sequential pattern identifying days of differentiation. LAM: laminin. The protocols show the concentration of each growth factors and a small molecule.

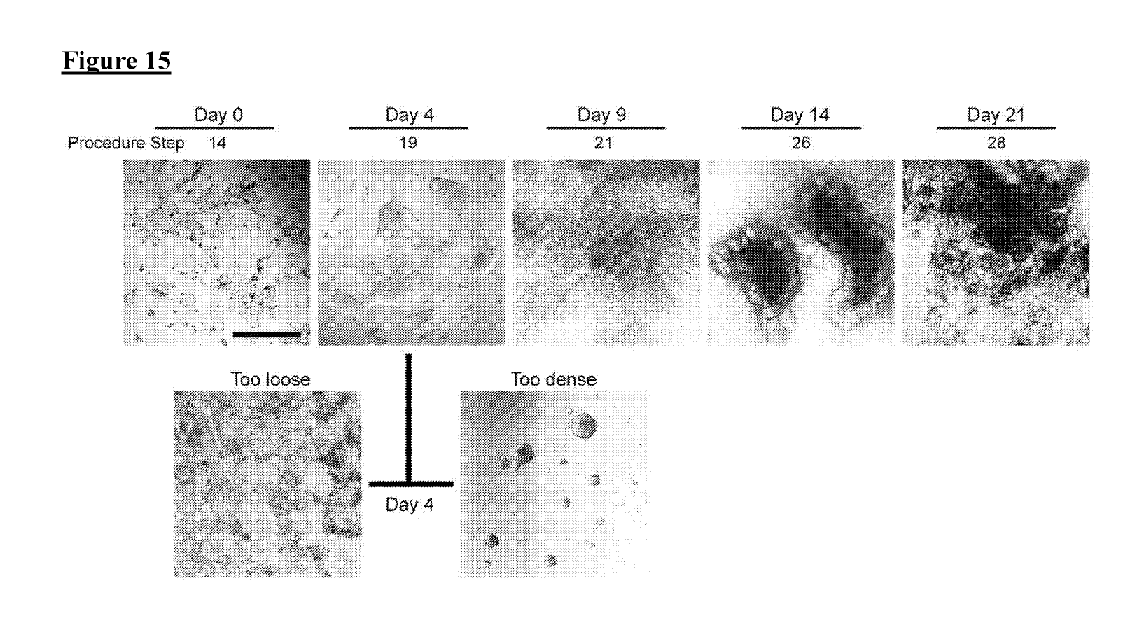

FIG. 15. Morphological changes of hPSCs at each step of differentiation. Representative bright field imaging at each step of differentiation. Day 0, undifferentiated hPSCs when differentiation is initiated. Day 4, late primitive streak stage. Day 9, nephron progenitor stage. Day 14, renal vesicle stage. Day 21, nephron stage. The optimal morphology of cells to proceed to activin A treatment on day 4 is the visual presence of loosely dense clusters. Representative bright field imaging of “too loose” or “too dense” clusters on day 4 is also shown. Scale bar: 100 μm. The scale bar is representative of all panels.

FIG. 16. Immunostaining for NPCs and nephrons. (a) Immunocytochemistry for SIX2 at day 9 of differentiation revealing NPCs. (b) Immunocytochemistry to identify nephron segments in 2D culture on day 21 of the differentiation. Scale bar: 50 (c) Immunohistochemistry to identify nephron segments in 3D culture with frozen sections on day 21 of differentiation. Scale bar: 50 μm. (d) Whole mount staining for nephrons in 3D culture (left: high magnification, scale bar: 50 μm, right: low magnification, scale bar: 100 μm). PODXL: podocalyxin (a podocyte marker). LTL: lotus tetragonolobus lectin (a proximal tubule marker). CDH1: cadherinl (also known as E-cadherin) (a loop of Henle and distal tubule marker). (e) Bright field imaging of an organoid in 3D culture on day 21. Arrows indicate a glomerular structure. Scale bar: 100 μm.

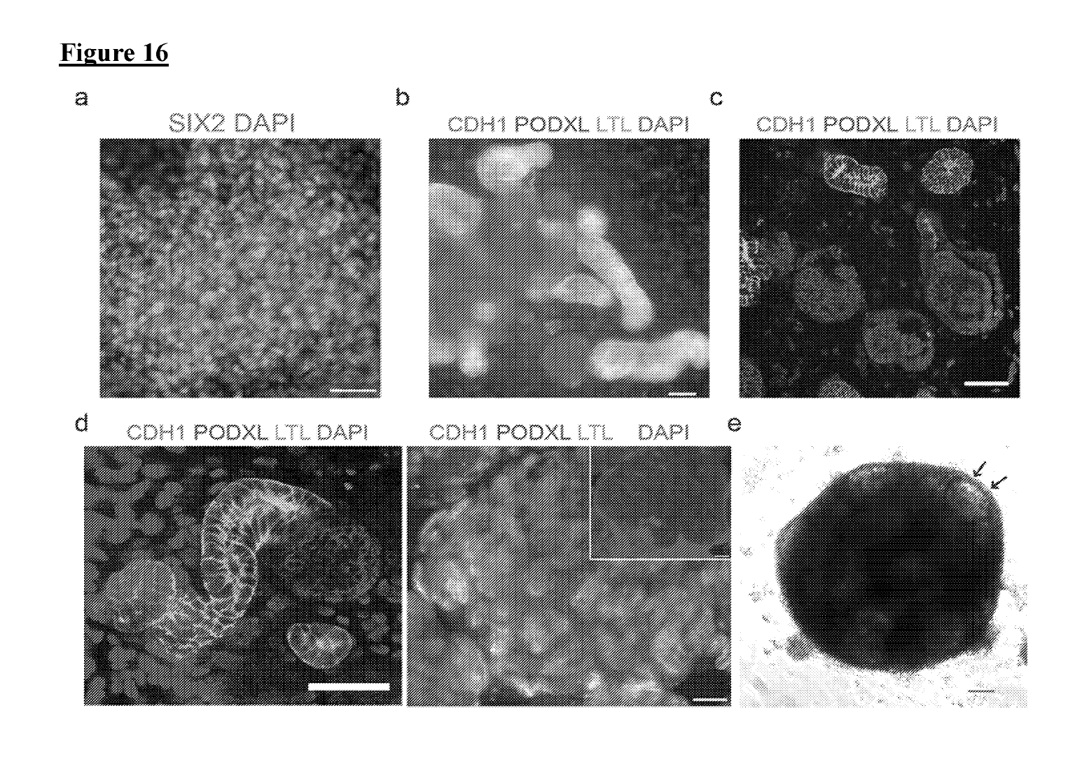

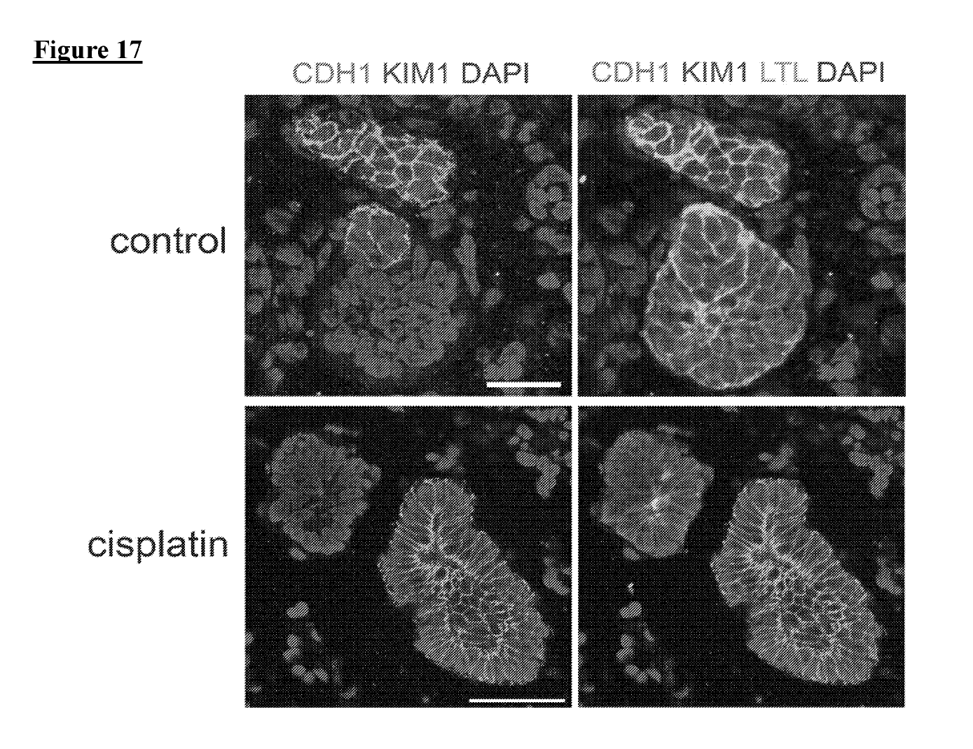

FIG. 17. Nephrotoxicity assay. Immunohistochemical staining for CDH1, KIM1, and LTL (lotus tetragonolobus lectin) in kidney organoids after 24 hours treatment with cisplatin 5 μM LTL+ tubules expressed KIM1 after the treatment, which is a marker for proximal tubular injury. Kidney organoids generated in 3D culture were treated with cisplatin 5 μM for 24 hours from day 23 to 24 of the differentiation. Organoids were fixed and analyzed on day 24. Scale bars: 50 μm. The scale bars are representative of the corresponding right panels.

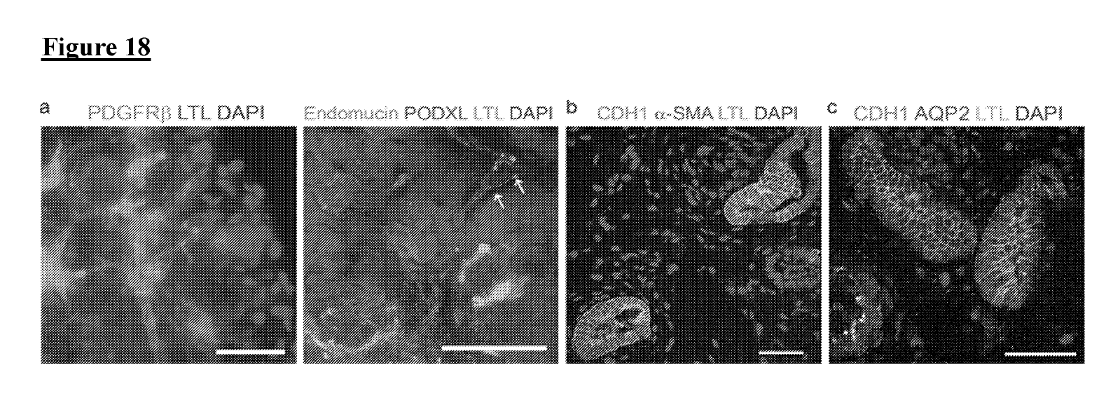

FIG. 18. Immunostaining for interstitial cells and connecting tubules/collecting ducts. (a) Immunostaining for PDGFRβ and endomucin in 3D kidney organoids. PEGFRβ was assessed by immunohistochemistry. Endomucin was evaluated by whole mount staining. Arrows indicate endomucin+endothelia in a glomerular structure. (b) Immunohistochemistry for α-SMA in 3D kidney organoids. There was a small population of α-SMA+ interstitial cells in kidney organoids. (c) Immunohistochemistry for aquaporin-2 (AQP2) in CDH1+ tubule in 3D kidney organoids. AQP2+ tubules were found in only CDH1+ tubules, indicating presence of connecting tubules/collecting ducts in kidney organoids. Scale bars: 50 μm.

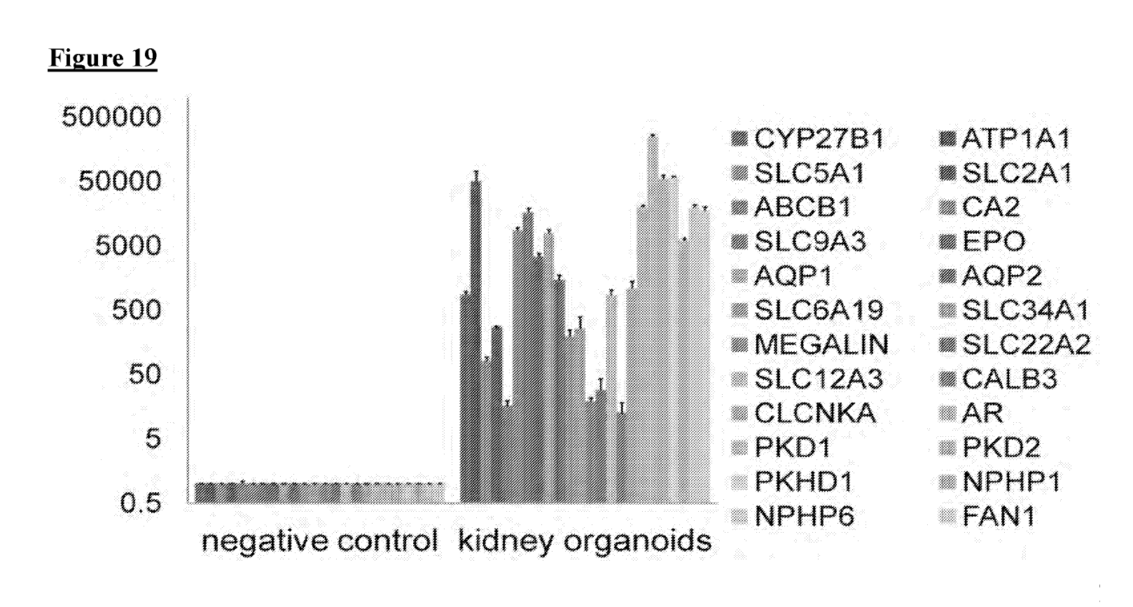

FIG. 19. Kidney organoids express transporters and functional proteins of kidneys in vivo.

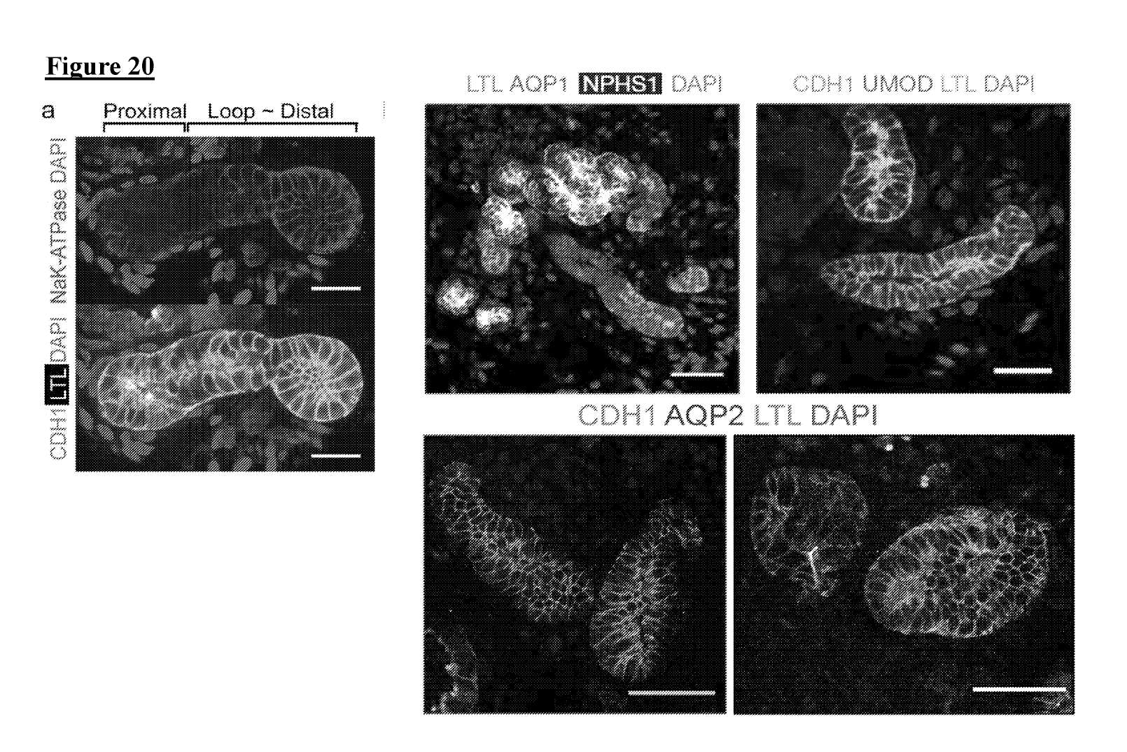

FIG. 20. Kidney Organoids contain multiple kidney compartments and express functional proteins of kidneys.

FIG. 21. Kidney Organoids contain multiple kidney compartments and express functional proteins of kidneys. Collectively, the organoids contain nephrons, collecting ducts, and interstitial cells including vasculature and myofibroblasts, meaning most of cell types of kidneys are included.

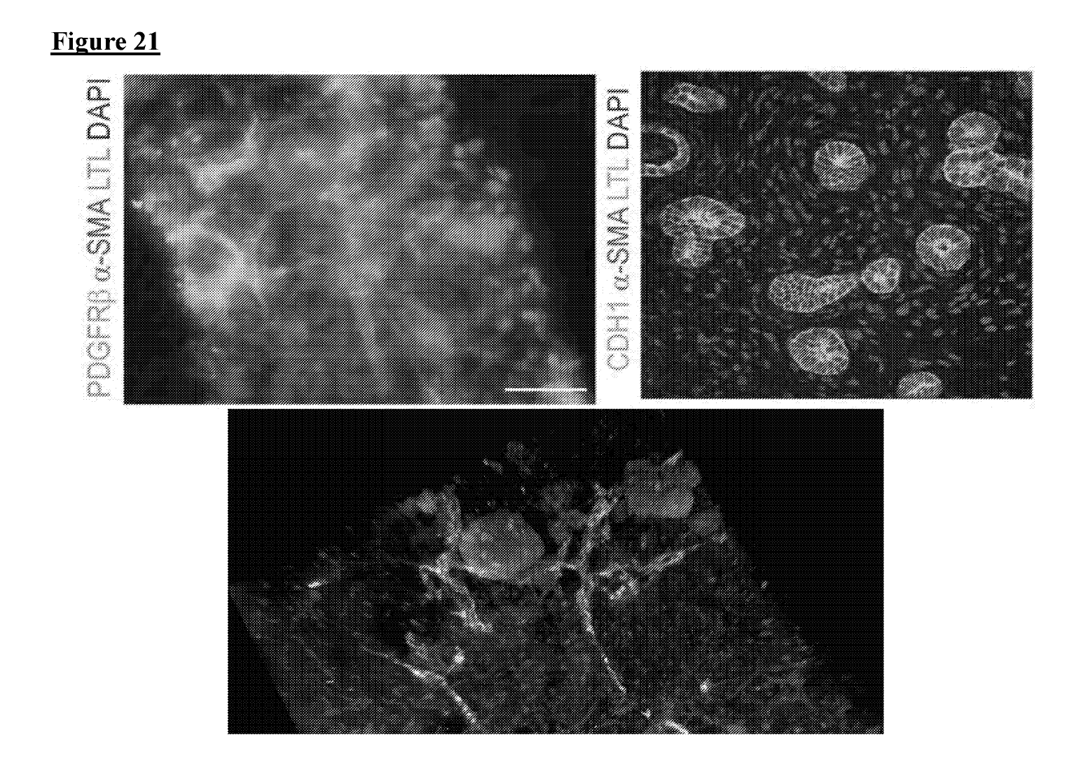

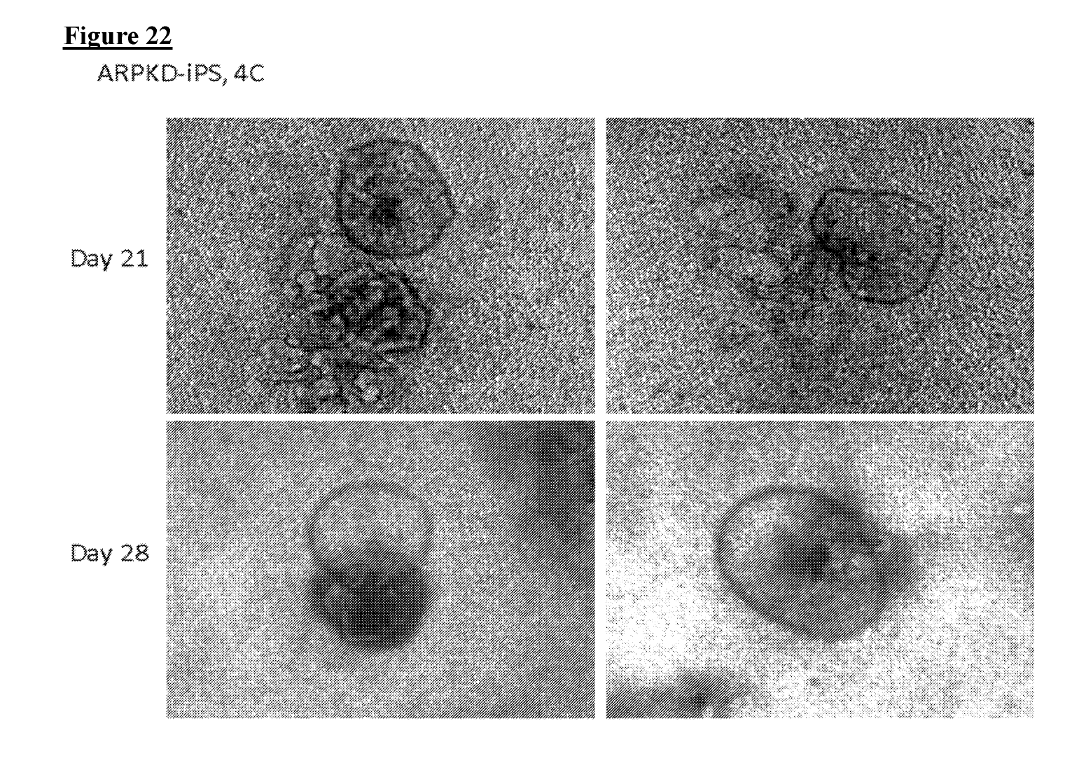

FIG. 22. Spontaneous cyst formation.

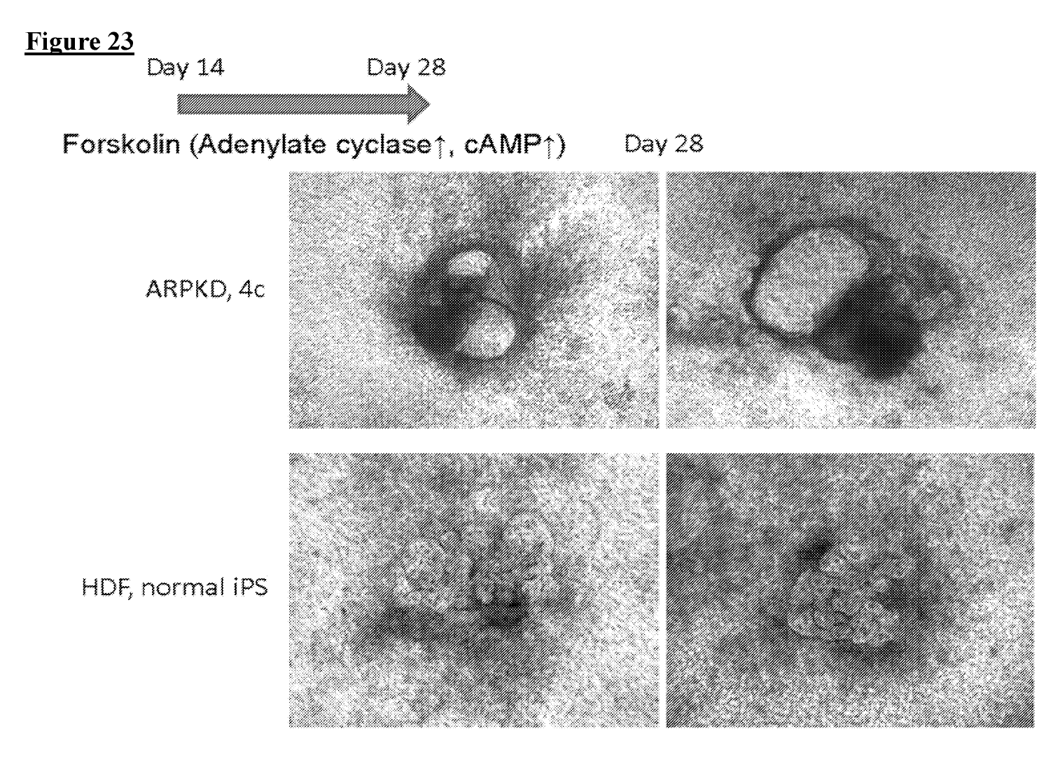

FIG. 23. Increased cAMP enhanced cyst formation.

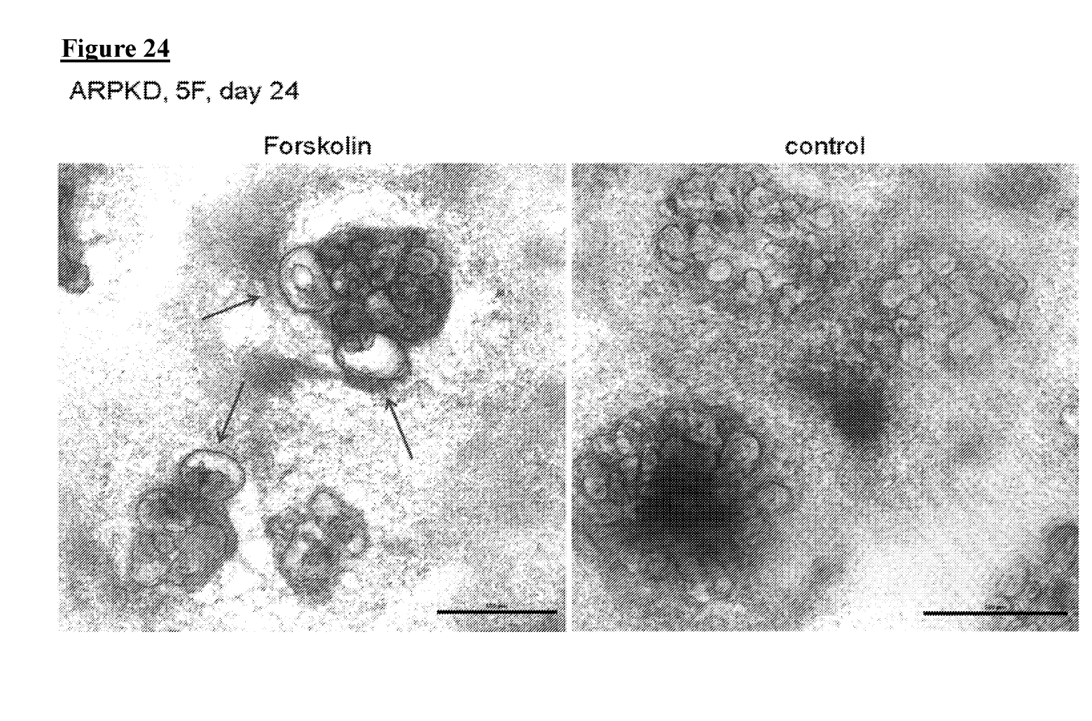

FIG. 24. Modeling autosomal recessive polycystic kidney disease (ARPKD) in nephron organoids.

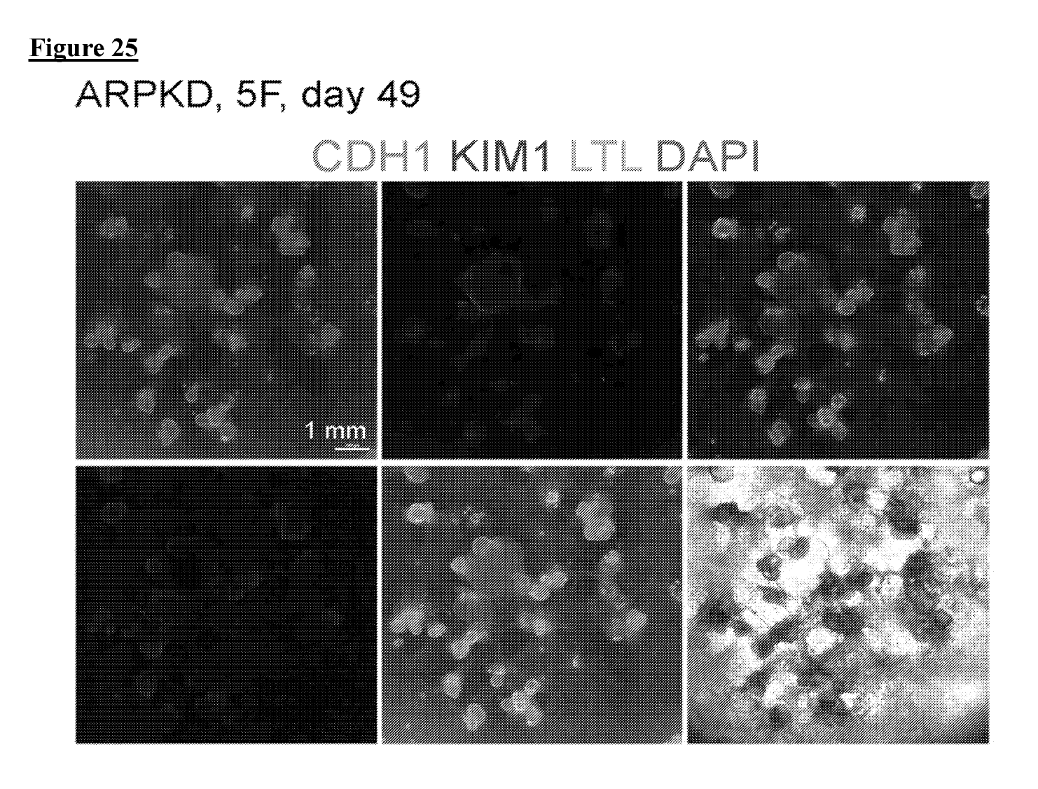

FIG. 25. Properties of autosomal recessive polycystic kidney disease (ARPKD). This shows that cysts are derived from distal nephrons (KIM1 negative) which is consistent with published studies.

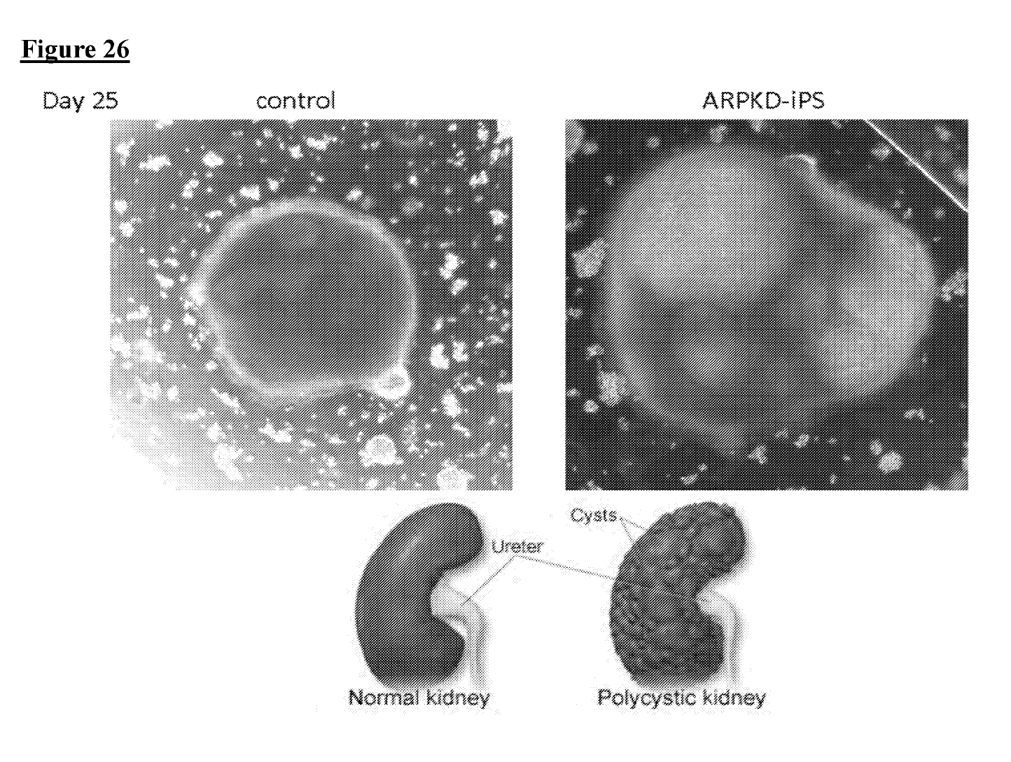

FIG. 26. Autosomal recessive polycystic kidney disease (ARPKD), organoid in 3D culture. This was done in 96-well plates. Cyst formation was 100% (24 in 24 organoids) in ARPKD-iPS with forskolin treatment. PKHD1 mutants were generated with CRISPR. The cystic phenotype appear from very early stage (day 22˜), meaning high-scale drug screening can be done, since media change will be much less frequent.

FIG. 27. Dedifferentiation and fibrosis. Modeling kidney fibrosis.

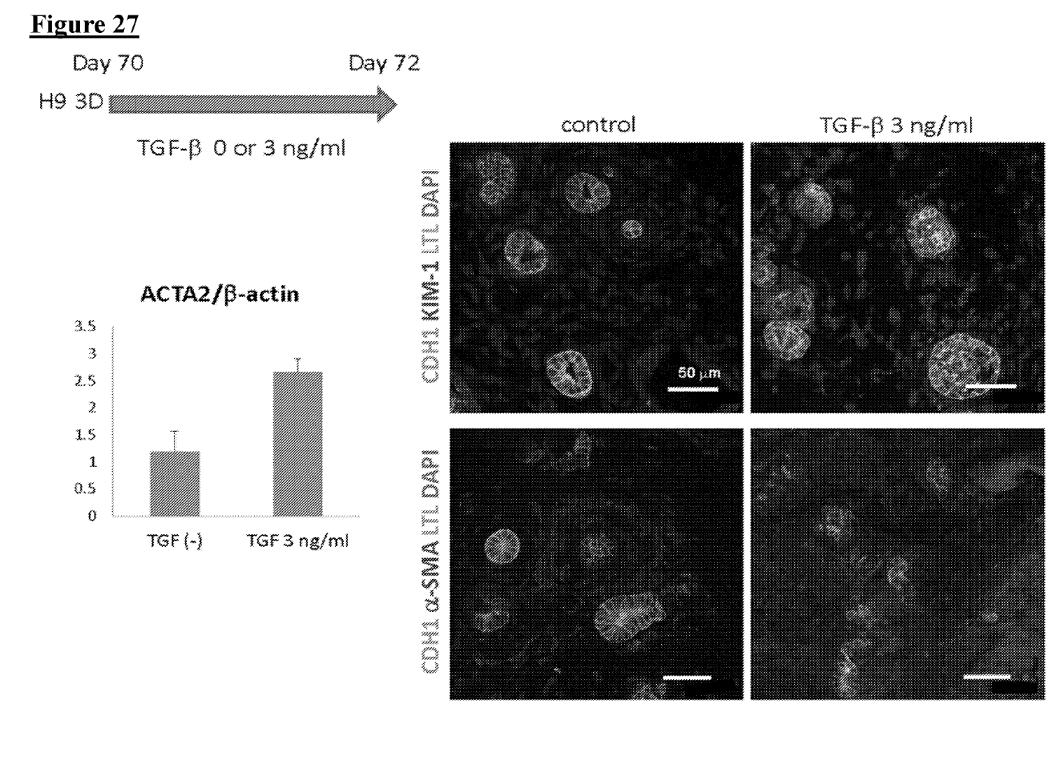

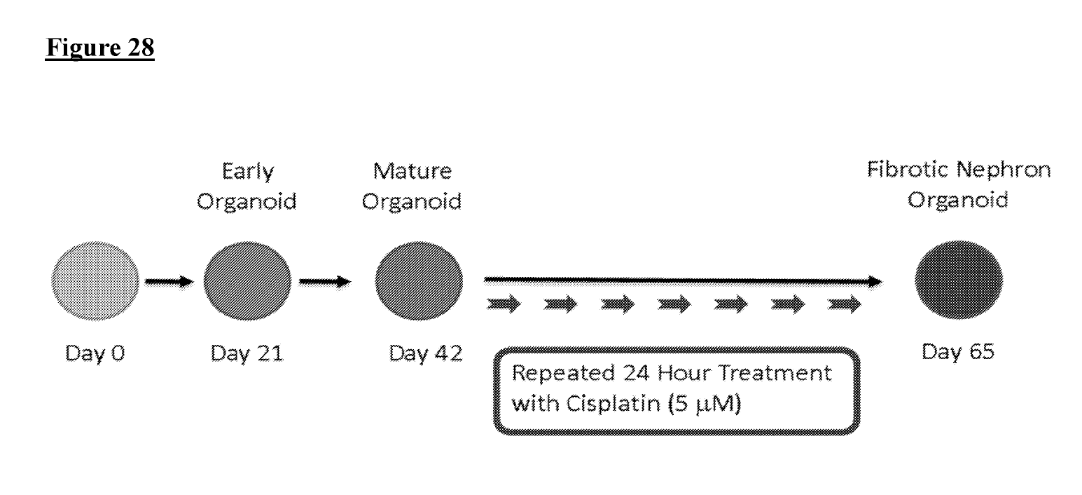

FIG. 28. Nephron Organoids: Fibrosis Modeling.

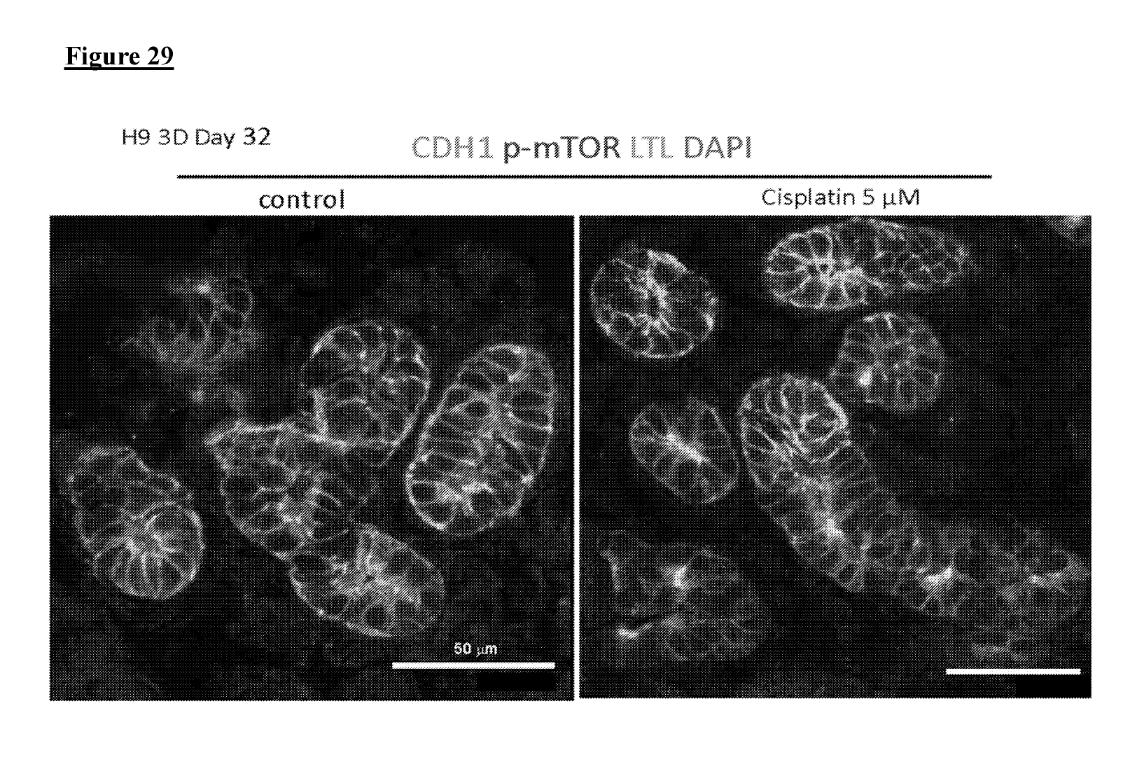

FIG. 29. Drug-induced kidney injury (Cisplatin 24 hours).

FIG. 30. Nephron Organoids: Fibrosis Modeling.

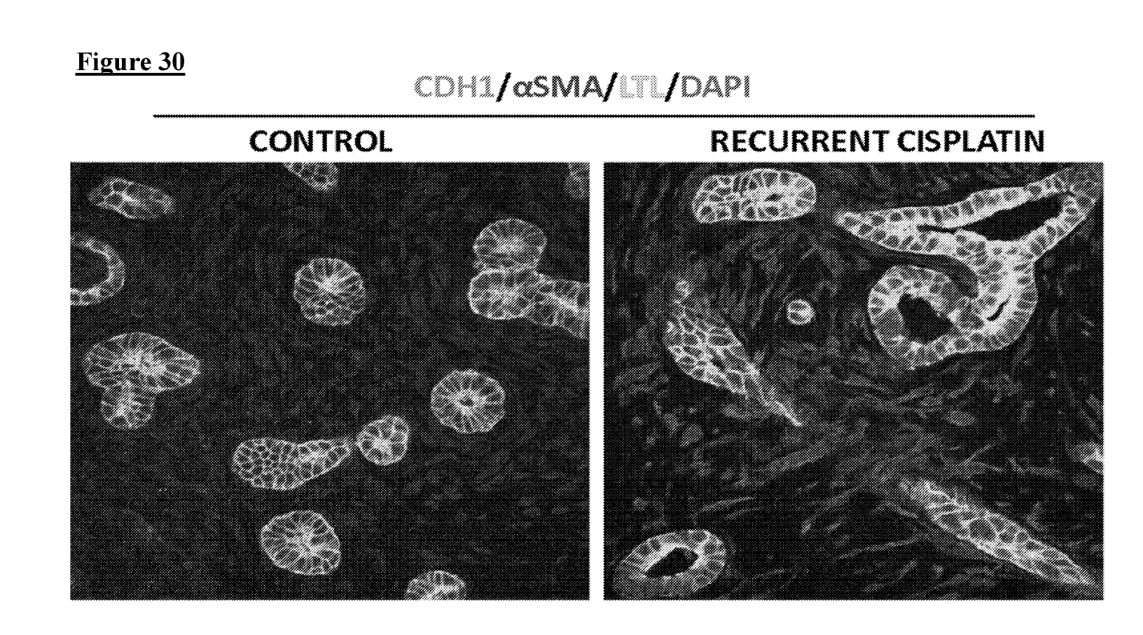

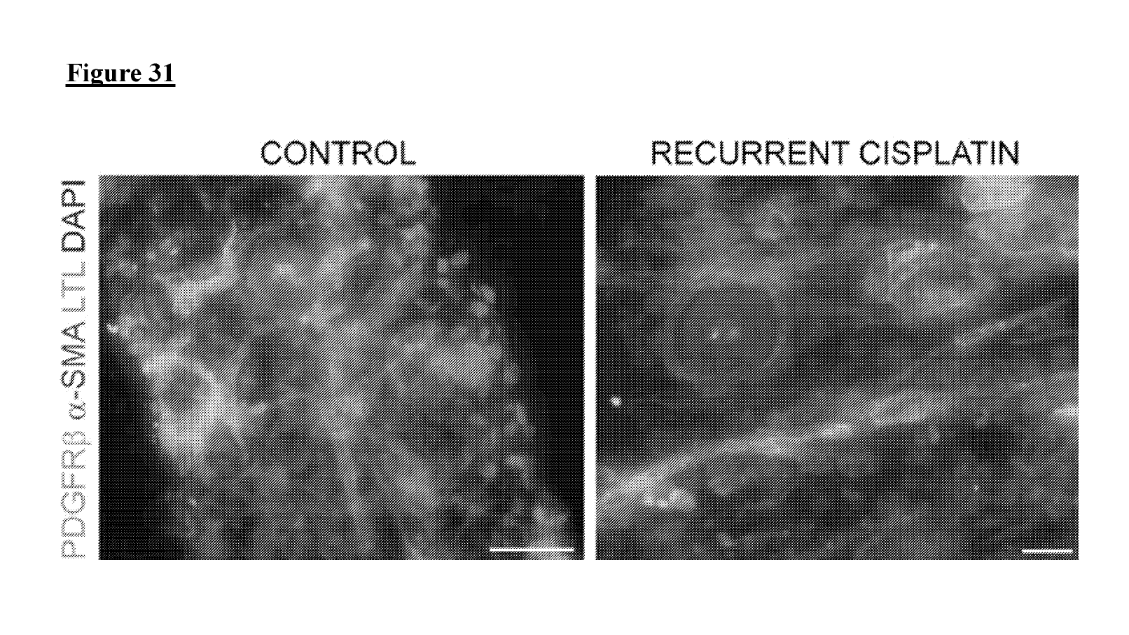

FIG. 31. Nephron Organoids: Fibrosis Modeling. Kidney fibrosis is very complicated pathological process with interaction between tubular cells and interstitial cells. Since the Inventors have both in organoids, the Inventors could make a novel human kidney fibrosis model. Current mouse models are quite different from humans. Fibrosis is the most important cause for chronic kidney disease.

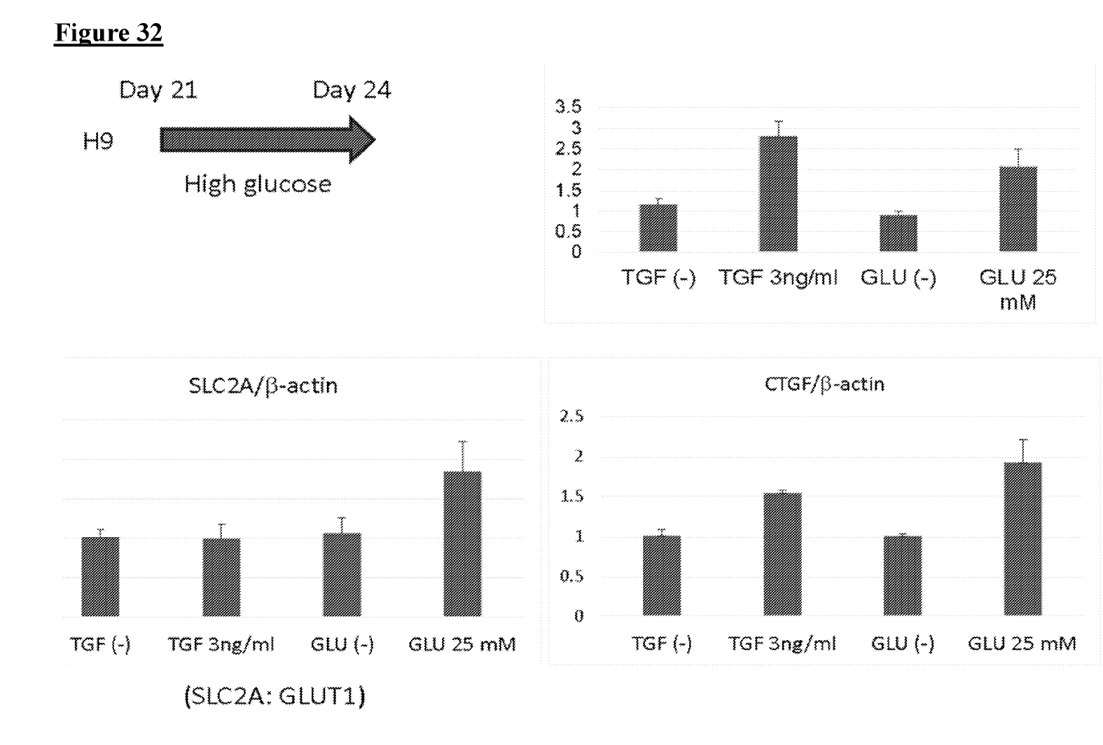

FIG. 32. Diabetic Model. High glucose treatment increased fibrosis pathways. Activation of glucose channel, SLC2A was also confirmed. Third rock is also interested in diabetic nephropathy models.

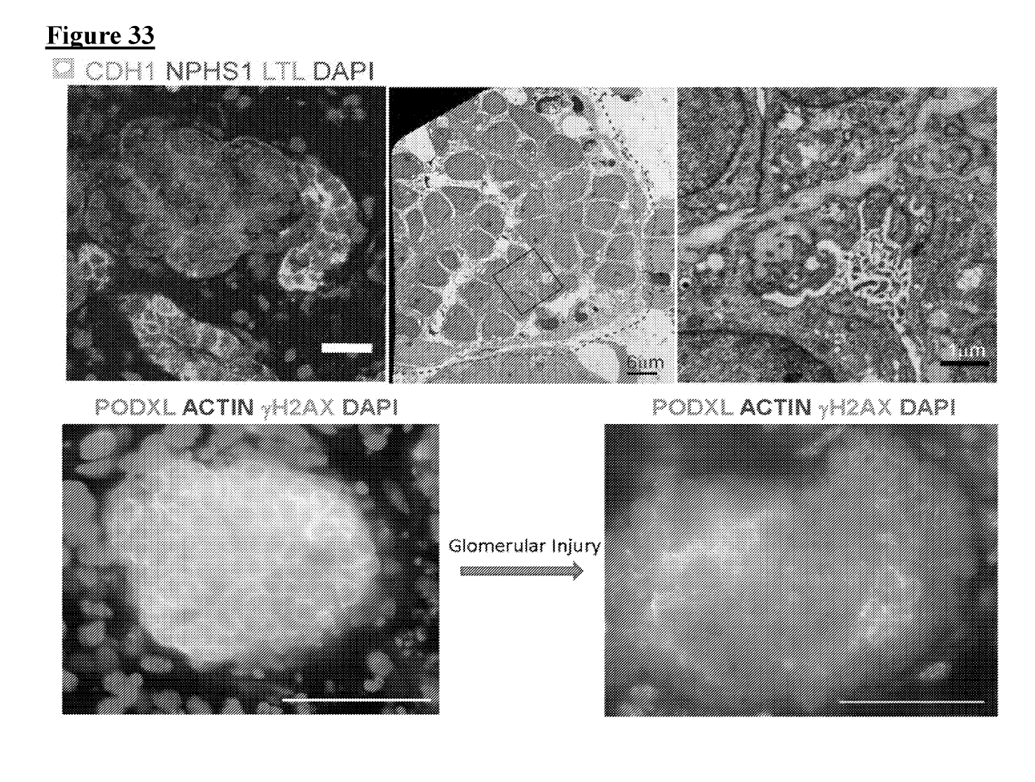

FIG. 33. Modeling glomerular diseases. The Inventors used puromycin and adriamycin which is known to induce FSGS (focal-segmental glomerulosclerosis). As shown by the bottom pictures, the structures of podocytes were disrupted like as FSGS.

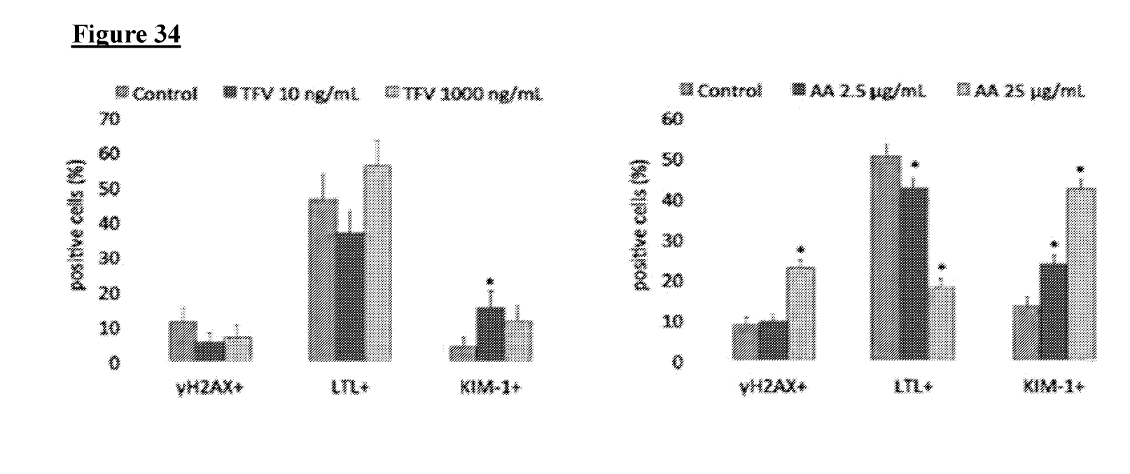

FIG. 34. AA causes dose-dependent upregulation of kidney damage biomarkers. Representative immunohistochemistry of aristolochic acid (AA) and tenofovir (TFV) treatments. Day 32 Kg-derived organoids. Al/AA treatments for 24H, while all TFV treatments for 48 H. Scale bars, 50 gm. Quantification determined as a percentage of DAP/+ cells. n=3, total DAP/+>1000 for each treatment. (*) indicates p-value<0.05.

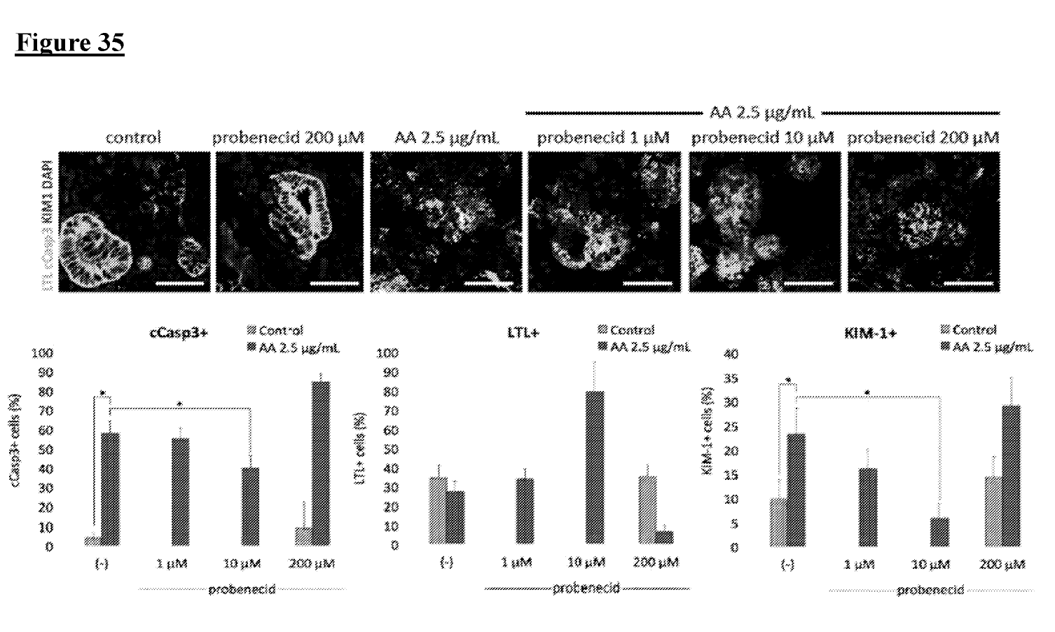

FIG. 35. OAT1 inhibition by probenecid protects proximal tubules against AA toxicity. Representative immunohistochemistry of probenecid treatments with AA 2.5 gg/mL. Day 38 Kg-derived organoids. All treatments for 48 H. Scale bars, 50 um. Quantification determined as a percentage of DAPI+ cells. n=3, total DAPI+>1000 for each treatment. (*) indicates p-value<0.05. Probenecid alone causes no damage to organoids. Probenecid 10 LIM provided strongest nephroprotection against AA.

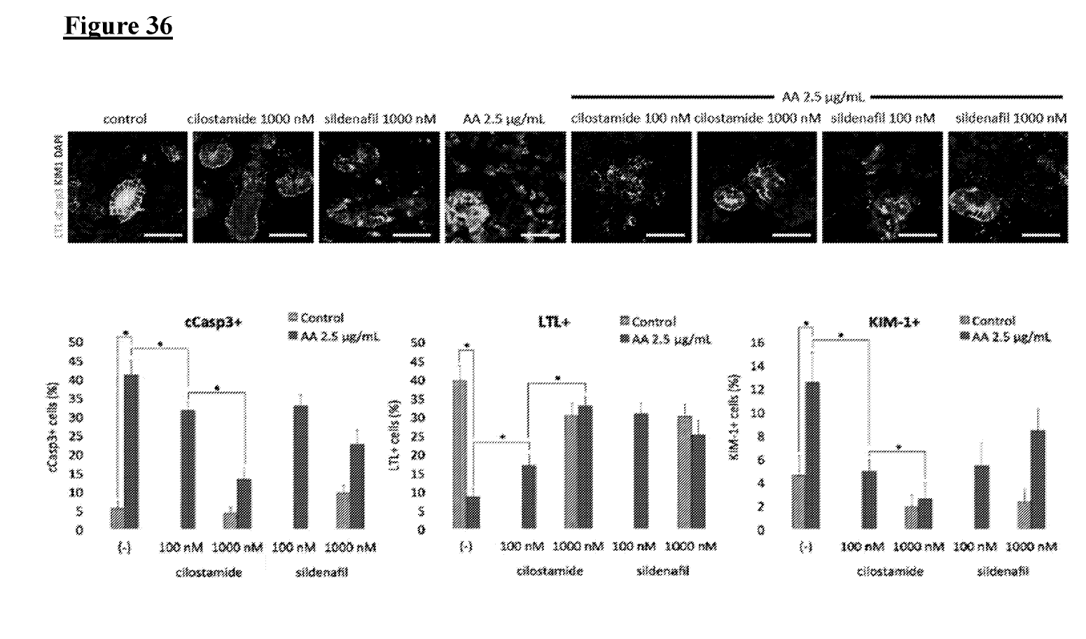

FIG. 36. Phosphodiesterase type 3 (PDE3) inhibition by cilostamide increases mitochondrial biogenesis and protection in AA-toxicity model. Representative immunohistochemistry of cilostamide and sildenafi/treatments with AA 2.5 gg/mL. Day 38 H9-derived organoids. All treatments for 48 H. Scale bars, 50 gm. Quantification determined as a percentage of DAPI+ cells. n=3, total DAPI+>1000 for each treatment. (*) indicates p-value<0.05.

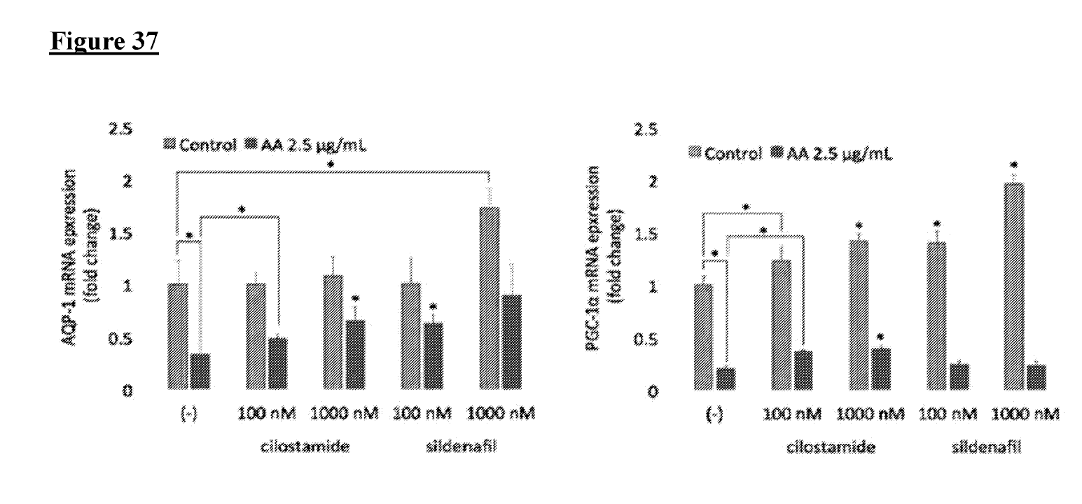

FIG. 37. Real-time quantitative PCR mRNA was extracted from organoid samples and confirmed with Nanodrop. cDNA library Was generated by reverse transcription. AQP-I is prominently expressed on proximal tubules. PGC1-a is the master regulator of mitochondrial biogenesis. Cilostamide shows dose-dependent nephroprotection against AA, significant preservation of proximal tubules. PGC-1a is downregulated by AA but protected by cilostamide. Each segment of nephrons expresses specific transporters which uptake specific drugs.Collectively, these data indicate that nephrons in organoids express specific functional transporters, which resulted in same drug responses to those observed in humans.

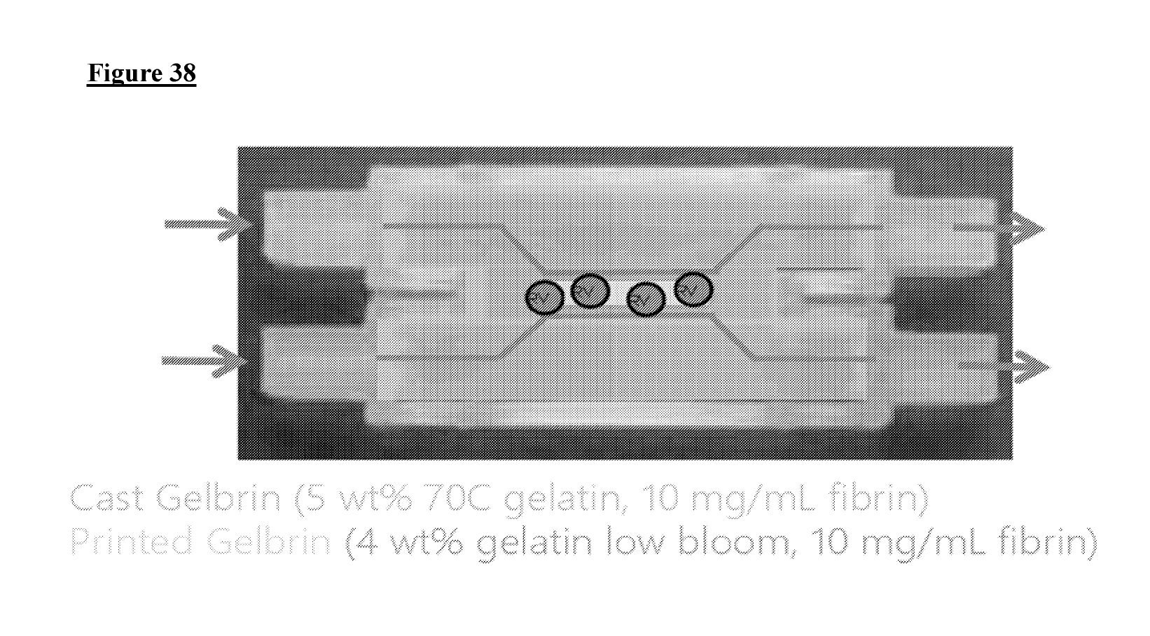

FIG. 38. Bioengineered didneys. The aforementioned cells and organoids can be adapted for in bioengineered designs, including transition to perfusion RV Day 15.

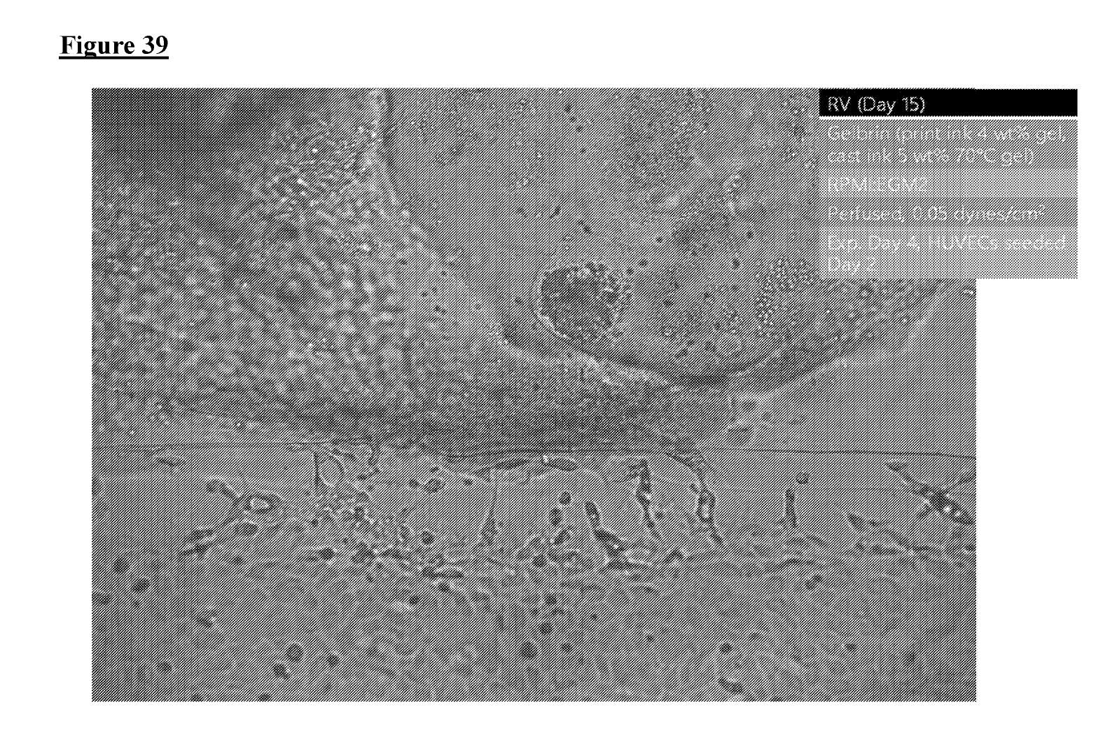

FIG. 39. RV-HUVEC interaction under perfusion.

FIG. 40. .RV-HUVEC interaction under perfusion

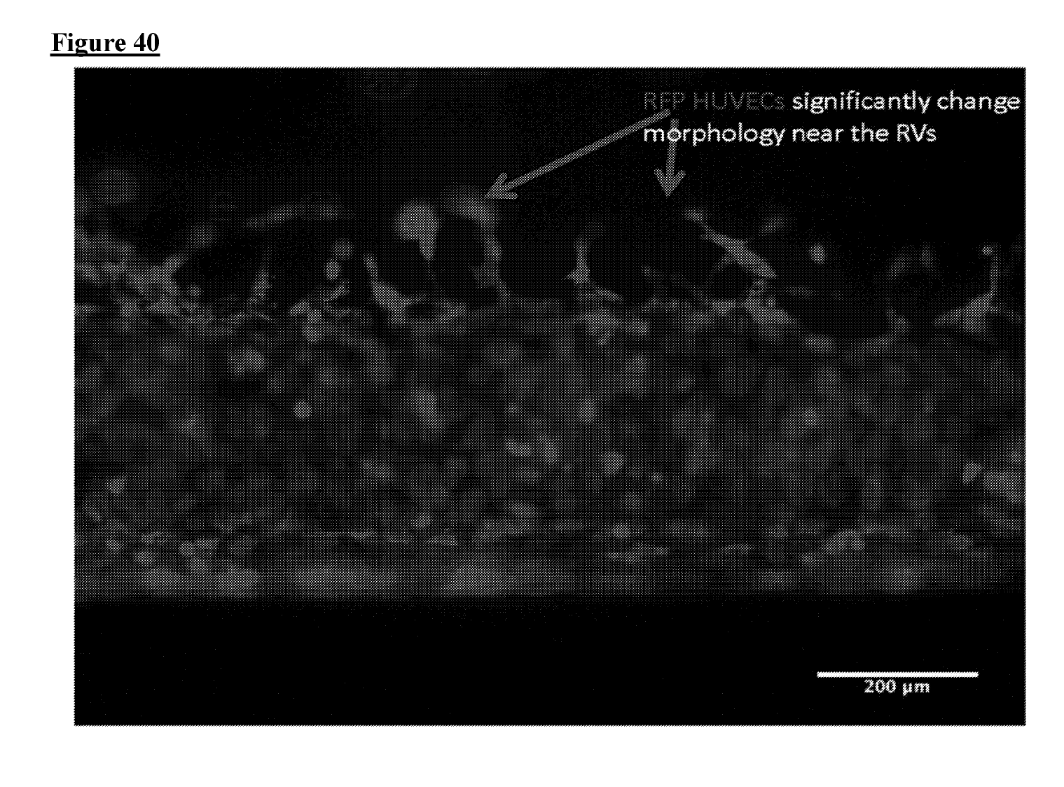

FIG. 41. RV with CD31+ population

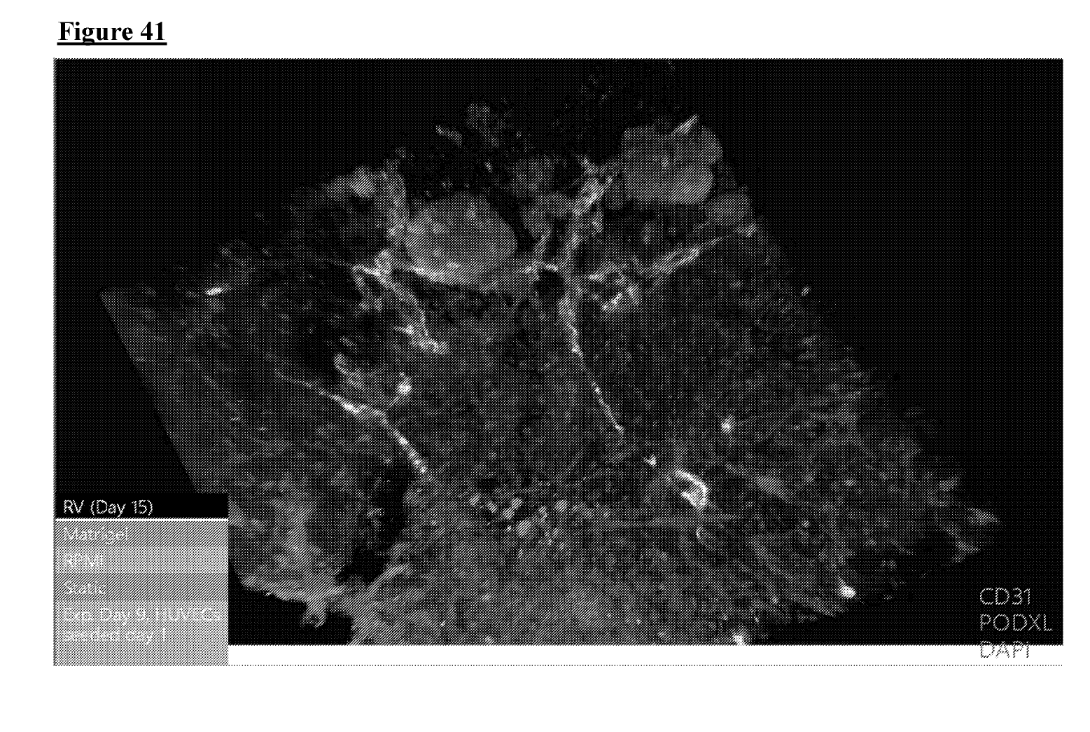

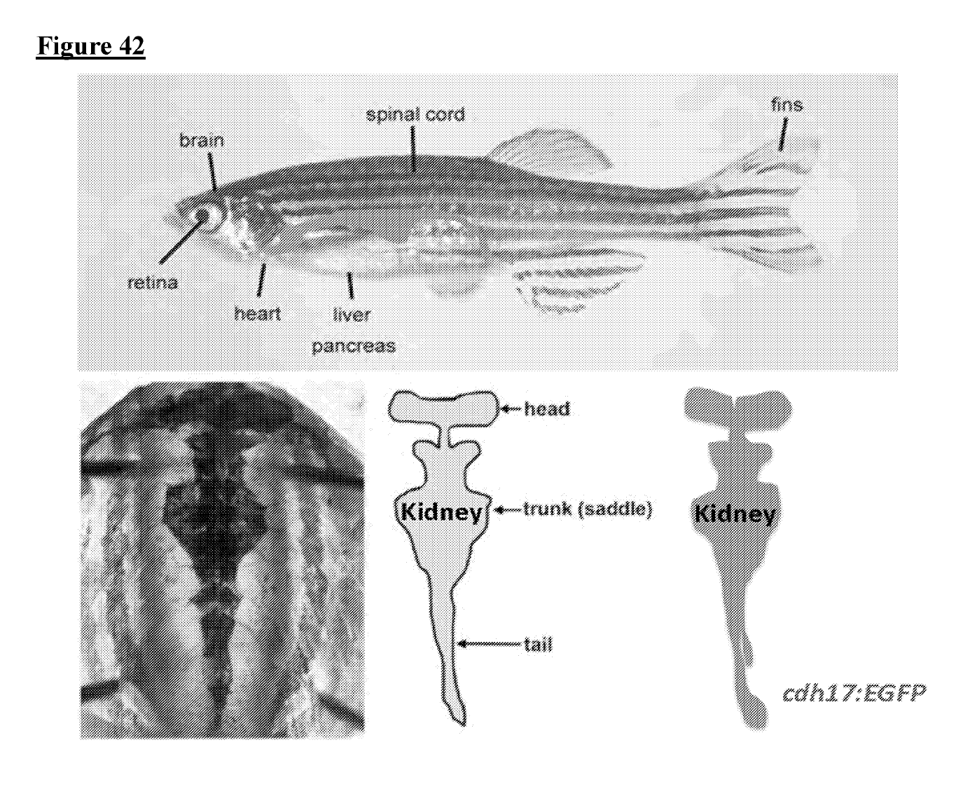

FIG. 42. Zebrafish kidney anatomy and cdh17:EGFP transgenic zebrafish

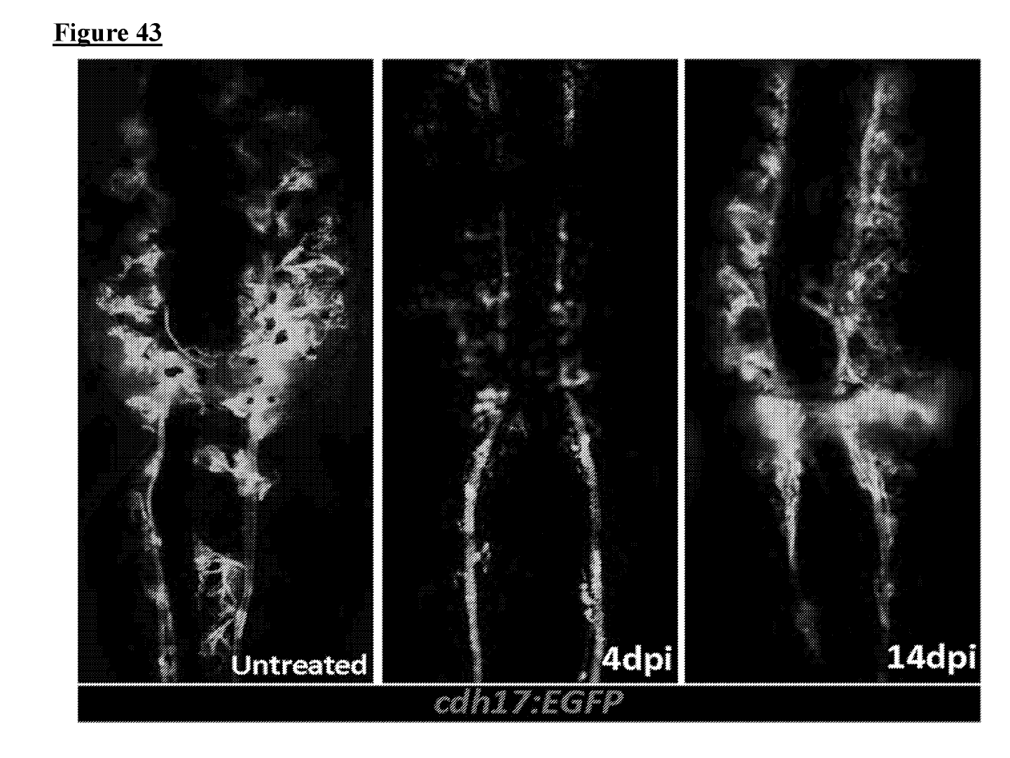

FIG. 43. Gentamicin-induced Kidney injury and nephrons regeneration in cdh17:EGFP transgenic zebrafish.

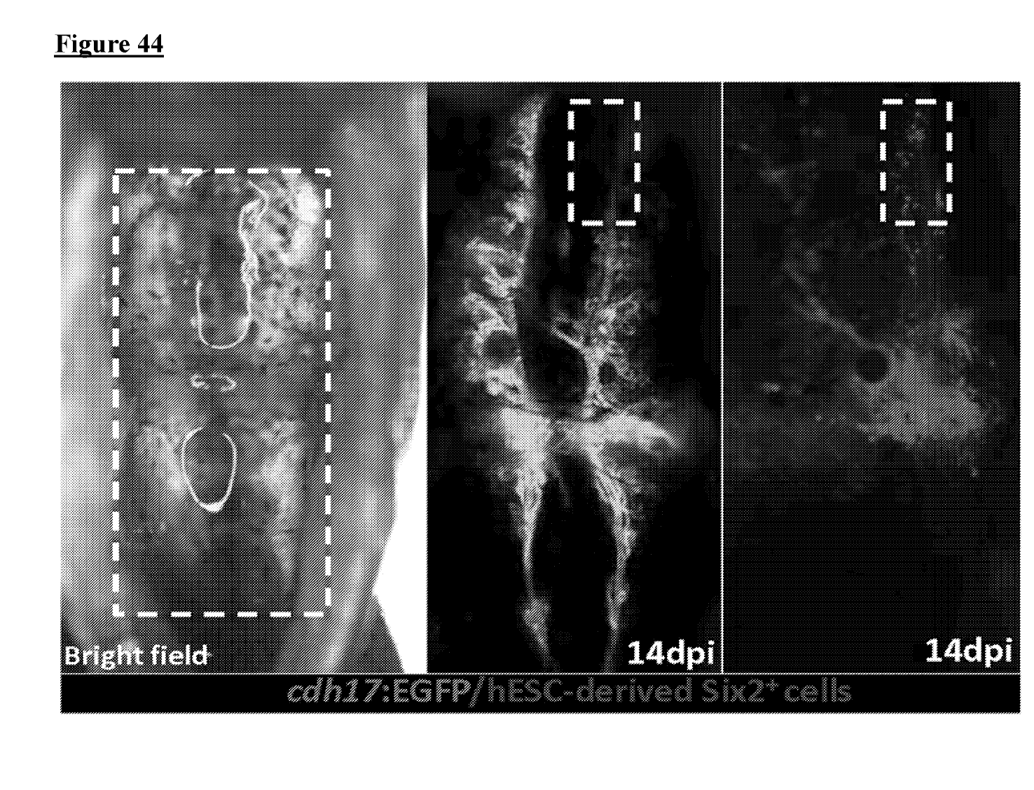

FIG. 44. Gentamicin-induced kidney injury and human kidney progenitor cells transplantation in cdh17:EGFP transgenic zebrafish.

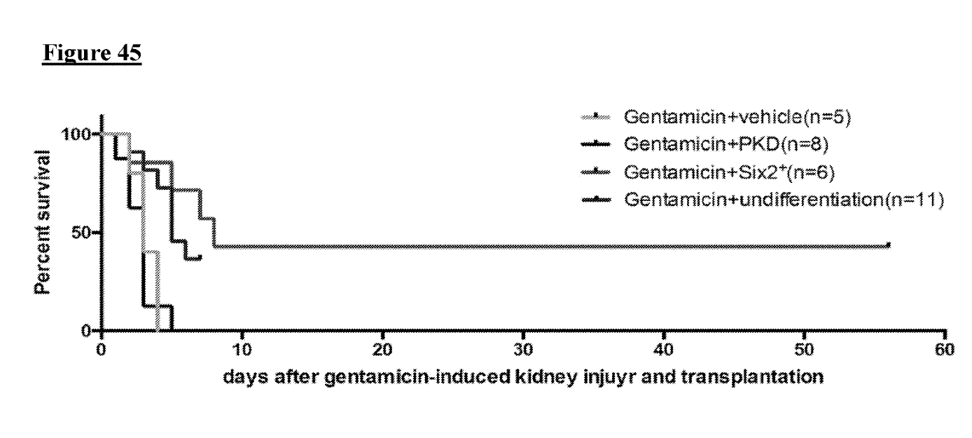

FIG. 45. Nephron progenitor cell transplantation improved survival of zebrafish after gentamicin-induced AKI. NPC transplantation improved survival after AKI.

### DETAILED DESCRIPTION

All references cited herein are incorporated by reference in their entirety as though fully set forth. Unless defined otherwise, technical and scientific terms used herein have the same meaning as commonly understood by one of ordinary skill in the art to which this invention belongs. Allen et al., Remington: The Science and Practice of Pharmacy 22^{nd} ed., Pharmaceutical Press (Sep. 15, 2012); Hornyak et al., Introduction to Nanoscience and Nanotechnology, CRC Press (2008); Singleton and Sainsbury, Dictionary of Microbiology and Molecular Biology 3^{rd} ed., revised ed., J. Wiley & Sons (New York, N.Y. 2006); Smith, March's Advanced Organic Chemistry Reactions, Mechanisms and Structure 7^{th} ed., J. Wiley & Sons (New York, N.Y. 2013); Singleton, Dictionary of DNA and Genome Technology 3^{rd} ed., Wiley-Blackwell (Nov. 28, 2012); and Green and Sambrook, Molecular Cloning: A Laboratory Manual 4th ed., Cold Spring Harbor Laboratory Press (Cold Spring Harbor, N.Y. 2012), provide one skilled in the art with a general guide to many of the terms used in the present application. For references on how to prepare antibodies, see Greenfield, Antibodies A Laboratory Manual 2^{nd} ed., Cold Spring Harbor Press (Cold Spring Harbor N.Y., 2013); Köhler and Milstein, Derivation of specific antibody-producing tissue culture and tumor lines by cell fusion, Eur. J. Immunol. 1976 July, 6(7):511-9; Queen and Selick, Humanized immunoglobulins, U.S. Pat. No. 5,585,089 (1996 December); and Riechmann et al., Reshaping human antibodies for therapy, Nature 1988 Mar. 24, 332(6162):323-7.

One skilled in the art will recognize many methods and materials similar or equivalent to those described herein, which could be used in the practice of the present invention. Indeed, the present invention is in no way limited to the methods and materials described.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise.

Human pluripotent stem cells (hPSCs) are attractive sources for regenerative medicine and disease modeling in vitro. To date, however, the protocols used for the differentiation of hPSCs into specific kidney cell types with high efficiency, without the need for less well defined inducers such as embryonic spinal cords, have eluded many researchers. Significant advances have been made within the past decade that draw upon the Inventors' knowledge of kidney development to differentiate PSCs into cells of the kidney lineage. By recapitulating metanephric kidney development in vitro, the Inventors generated nephron progenitor cells (NPCs) with ˜80-90% purity within 9 days, without subpopulation selection during the directed differentiation protocol. hPSC-derived NPCs possess the developmental potential of their in vivo counterparts, forming renal vesicles that self-pattern into nephron structures. In both 2D and 3D culture, NPCs form kidney organoids containing epithelial nephron-like structures expressing markers of podocytes, proximal tubules, loops of Henle, distal tubules in organized, continuous structures that resemble the nephron in vivo.

A recent study revealed that the origin of the metanephros is distinct from the ureteric bud or pro/mesonephric lineages. They showed that the metanephros arises from the posterior intermediate mesoderm, whereas the ureteric bud and the pro/mesonephros is derived from anterior intermediate mesoderm. Therefore, the Inventors hypothesized that the specific induction of posterior intermediate mesoderm cells from hPSCs would greatly facilitate the induction of NPCs and avoid contamination with pronephric or mesonephric cells. Previous studies revealed that locations in the primitive streak define the subsequent differentiation into each segment of mesoderm i.e. paraxial, intermediate or lateral plate mesoderm. In addition, the timing of cell migration from the primitive streak defines the anterior-posterior axis in mesoderm, suggesting that the late stage of the primitive streak induces posterior mesoderm. The Inventors optimized the time of treatment with the GSK-3β inhibitor, CHIR99201 (CHIR), an inducer of the primitive streak, to induce late stage primitive streak. Additionally, the Inventors employed BMP4 inhibitors, as high BMP4 activity induces more posterior aspects of the primitive streak, which develops into lateral plate mesoderm. With this approach, the Inventors found a highly efficient protocol to induce SIX2+SALL1+WT1+PAX2+EYA1+ NPCs from both human ESCs and iPSCs with 80-90% efficiency within 9 days of differentiation. After the induction of NPCs, the Inventors transiently treated cells with CHIR (3 μM), generating multi-segmented nephron structures with characteristics of podocytes, proximal tubules, loops of Henle, and distal tubules sequenced in a self-assembled tubule in a manner that reflects normal nephron structure. Further analyses of other organoid compartments revealed CDH1+AQP2+ tubules and PDGFRI3+, endomucin+, or α-SMA+ interstitial cells in the kidney organoids (FIG. 7). Collectively, the Inventors' protocols generated kidney organoids consisting of multiple kidney compartments with cellular proportion similar to that of in vivo kidneys where nephrons occupy nearly 90% of renal cortex.

The protocols to differentiate hPSCs into NPCs and kidney organoids provide novel platforms in vitro to study human kidney development and developmental disorders, inherited kidney diseases, kidney injury, nephrotoxicity testing, and kidney regeneration. In addition, the organoids provide systems in vitro for the study of intracellular and intercellular kidney compartmental interactions using differentiated cells. Since the protocols were derived to follow the steps of kidney development as the Inventors know them in vivo, the Inventors can induce intermediate cell populations at each step of differentiation: late mid primitive streak, posterior intermediate mesoderm, NPCs, pre-tubular aggregates, renal vesicles, and nephrons (FIG. 1). Therefore, the organoids may enable the study of human kidney development and kidney congenital abnormalities by evaluating the cells at each step of differentiation. An important application will be to study inherited kidney diseases. There are more than 160 inherited kidney diseases with specific identified mutations. By generating iPSCs from patients with inherited kidney diseases, and producing kidney organoids from these cells, the pathogenesis of inherited kidney diseases can be explored. Moreover, it is also possible to study inherited kidney diseases by introducing targeted mutations with CRISPR/Cas9 genome editing in hPSCs and taking advantage of comparisons of organoids from mutated and parental lines with otherwise uniform genetic background. These approaches will enable the analysis of inheritable disease pathophysiology and allow for drug screening in vitro in order to find new therapeutic approaches. Another application of the kidney organoids will be to test nephrotoxicity of drugs in predictive toxicology based on genotypic characteristics of an individual. Since the kidney organoids contain multiple cell types, reflecting sequential segments of the nephron from podocytes to distal tubules, it will be possible to assign drug toxicity to specific nephron segments. Already, results on hPSCs derived from patients with autosomal recessive polycystic kidney disease (ARPKD) (FIGS. 24-26), fibrosis organoids (FIGS. 27-31), further including potential uses for modeling diabetes (FIG. 32) and drug toxicity (FIG. 33-36). These results demonstrate the platform's utility and flexibility.

The maintenance of a differentiated phenotype in vitro will also allow for cellular biochemical analyses and the study of inter-compartmental interactions in ways that will likely more closely mimic the status in vivo than typical cell culture studies where the cells are generally dedifferentiated. The presence of CDH1+AQP2+ tubules and PDGFRβ+, endomucin+, or α-SMA+ interstitial cells, will permit studies of nephron-interstitial cell interactions. Ultimately, the protocol has the potential to serve as a foundation to provide organoids for kidney regenerative therapies.

In comparing the Inventors' protocol to previous published protocols to induce kidney lineage cells, there are many differences in efficiency, specificity, and simplicity. The Inventors' protocols yield NPCs, with much higher induction efficiency, from both hESCs and hiPSCs when compared to previous studies, including the Inventors' own. High induction efficiency at each step of differentiation, simultaneously, indicates high specificity of kidney induction. One study reported relatively high efficiency (˜60%) of SIX2+ cell induction with embryoid body formation, yet co-culture with mouse embryonic spinal cords is required to generate kidney epithelial cells while the Inventors' protocols use monolayer culture and chemically defined components. The Inventors were able to generate NPCs and organoids using fully defined conditions without the addition of any non-purified non-human factors, which is desirable for regenerative utility in humans. In addition, the Inventors' protocols use 96-well, round bottom, ultra-low attachment plates to generate 3D kidney organoids, which enables mass production of kidney organoids, while the other protocols to generate organoids require pelleting cells in eppendorf tubes or co-culture with mouse embryonic spinal cords. As previous studies have shown that the efficiency of the same differentiation protocol differs in separate hPSC lines, adjustments must be made to achieve similar results in varying lines. The Inventors define how to adjust the protocol for different lines of hPSCs, which further facilitates the applicability of the Inventors' differentiation protocols. The dose of growth factors can greatly influence the costs of the directed differentiation protocols. The Inventors were able to use lower doses of FGF9 than those used in the excellent protocol of Takasato et al. This has substantial financial advantages at the present time.

Other protocols result in generation of CD31+ endothelia-like cells and CDH1+GATA3+ collecting duct-like cells which on the surface can be perceived as a shortcoming of the Inventors' protocol. On the other hand the Inventors have now documented the presence of CDH1+AQP2+ tubules and PDGFRβ+, endomucin+, or α-SMA+ interstitial cells in the kidney organoids (FIG. 7). The cells expressing these markers require more characterization and one of the Inventors' goals is to understand how these populations might be enhanced in the Inventors' protocol. Ultimately non-nephron cells are useful to establish a multi-compartment environment in the kidney organoids potentially leading to vascularization of glomerular and tubulo-interstitial structures. The ability to generate NPCs with high efficiency and ultimately multi-segmented nephrons serves as a very good starting point for subsequent bioengineering of functional kidney tissues.

Described herein is a method for generating metanephric mesenchyme, including providing a quantity of human pluripotent stem cells (“hPSCs”), generating late primitive streak cells, inducing formation of posterior intermediate mesoderm cells, and differentiating into metanephric mesenchyme cells. In other embodiments, the human pluripotent stem cells are human embryonic stem cells (“hESCs”). In other embodiments, the human pluripotent stem cells are human induced pluripotent stem cells (“hiPSCs”). In other embodiments, generating late primitive streak cells includes culturing hPSCs in CHIR99021 for about 3-5 days. In other embodiments, this includes about 4 days. In various embodiments, the concentration of CHIR99021 is about 3, 4, 5, 6, 7, 8, 9, 10, 11, 12, 13, 14, 15 μM. In various embodiments, the concentration of CHIR99021 is about 8-10 μM. In other embodiments, the method further includes addition of noggin In various embodiments, the concentration of noggin is about 1, 2, 3, 4, 5, 6, 7, 8, 9, 10 ng/ml. In various embodiments, the concentration of noggin is about 5 ng/ml. In other embodiments, inducing formation of posterior intermediate mesoderm cells includes culturing in the presence of activin for about 2-4 days. In other embodiments, this includes about 3 days. In various embodiments, the concentration of activin is about 5-10, 10-20-30 ng/ml. In various embodiments, the concentration of activin is about 10 ng/ml. In other embodiments, differentiating into metanephric mesenchyme cells includes addition of FGF9. In various embodiments, the concentration of FGF9 is about 5-10, 10-20-30 ng/ml. In various embodiments, the concentration of FGF9 is about 10 ng/ml. In other embodiments, the metanephric mesenchyme lineage cells are further differentiated into nephronic progenitor cells (NPCs) by addition of CHIR99021. In various embodiments, the concentration of CHIR99021 is about 1, 2, 3, 4, 5, 6, 7, 8, 9, 10 μM. In various embodiments, the concentration of CHIR99021 is about 5 μM. In other embodiments, late primitive streak cells express one or more of: T and TBX. In other embodiments, posterior intermediate mesoderm cells express one or more of: WT1 and HOXD11. In other embodiments, metanephric mesenchyme lineages cells express one or more of: SIX2, SALL1, WT1, and PAX2. In other embodiments, NPCs express one or more of: SIX2, SALL1, WT1, PAX2, and EYA1. In other embodiments, differentiation into metanephric mesenchyme cells is at least 50, 60, 70% or more efficient. In other embodiments, differentiation into metanephric mesenchyme cells is at least 70, 80, 90% or more efficient.

For example, hPSCs can be dissociated into single cells and maintained in a culture medium supplemented with the ROCK inhibitor Y27632 and optionally, FGF2 (10 ng/ml). Cells that are about 30, 40, 50, 60, or 70% confluent are then cultured in basic differentiation medium supplemented with CHIR99021 (8-10 μM) for 4 days to induce late primitive streak cells Noggin (5 ng/ml) was also used for hiPSC differentiation in addition to CHIR (10 To induce posterior intermediate mesoderm, cells were then cultured in Advanced RPMI+1X L-GlutaMAX+activin (10 ng/mL) for 3 days. For induction of nephron progenitor cells, the media was then changed to Advanced RPMI+1X L-GlutaMAX+FGF9 (10 ng/ml) for 7 days. CHIR (3 μM) can be added to the media from day 9 to 11 of differentiation to induce renal vesicles. On day 14, cells were switched to the basic differentiation medium and cultured for an additional 7 to 14 days (total of 21 to 28 days). The medium was replaced every 2 or 3 days. A variety of growth factors and small molecules were tested for differentiation. For organoid formation, hPSCs on day 9 of differentiation, which represents metanephric mesenchyme cells, arare dissociated resuspended in the basic differentiation medium supplemented with CHIR (3 μM) and FGF9 (10 ng/mL) and cultured at 37° C., 5% CO₂ for 2 days. The medium is then changed to the basic differentiation medium supplemented with FGF9 10 ng/mL and cultured for 3 more days. After that, the organoids were cultured in basic differentiation medium with no additional factors for 7-21 days (a total of 21-35 days).

Further described herein is a composition of metanephric mesenchyme cells generated by a method for generating metanephric mesenchyme, including providing a quantity of human pluripotent stem cells (“hPSCs”), generating late primitive streak cells, inducing formation of posterior intermediate mesoderm cells, and differentiating into metanephric mesenchyme cells. In other embodiments, the human pluripotent stem cells are human embryonic stem cells (“hESCs”). In other embodiments, the human pluripotent stem cells are human induced pluripotent stem cells (“hiPSCs”). In other embodiments, generating late primitive streak cells includes culturing in CHIR99021 for about 3-5 days. In other embodiments, the method further includes addition of Noggin. In other embodiments, inducing formation of posterior intermediate mesoderm cells includes culturing in the presence of activin for about 2-4 days. In other embodiments, differentiating into metanephric mesenchyme cells includes addition of FGF9. In other embodiments, the metanephric mesenchyme lineage cells are further differentiated into nephronic progenitor cells (NPCs) by addition of CHIR99021. In other embodiments, late primitive streak cells express one or more of: T and TBX. In other embodiments, posterior intermediate mesoderm cells express one or more of: WT1 and HOXD11. In other embodiments, metanephric mesenchyme lineages cells express one or more of: SIX2, SALL1, WT1, and PAX2. In other embodiments, NPCs express one or more of: SIX2, SALL1, WT1, PAX2, and EYA1. In other embodiments, differentiation into metanephric mesenchyme cells is at least 50% efficient. In other embodiments, differentiation into metanephric mesenchyme cells is at least 70% efficient. A composition of nephron progenitor cells generated by the described method.

Described herein is a method of generating kidney organoids, including providing a quantity of nephron progenitor cells (“NPCs”), and culturing the NPCs in a suspension culture for at about 11 days. In other embodiments, the method includes addition of one or more of: CHIR99021 and FGF9. In other embodiments, the kidney organoids comprise one or more cell types selected from: podocyte-like cells, proximal tubules, descending limbs of Henle, thick ascending limbs of Hendle, and distal convoluted tubules. In other embodiments, podocyte-like cells express one or more of: NPHS1+, PODXL+, and WT1+. In other embodiments, proximal tubules express one or more of: LTL+ and AQP1+. In other embodiments, descending limbs of Henle express one or more of: CDH1+ and AQP1+. In other embodiments, thick ascending limbs of Henle express one or more of CDH1+ and UMOD+. In other embodiments, distal convoluted tubules express one or more of CDH1+UMOD−. In other embodiments, NPCs are derived from human pluripotent stem cells (“hPSCs”). In other embodiments, hPSCs are derived from a patient suffering a disease mutation. In other embodiments, hPSCs have been genomically edited using CRISPR.

Also described herein is a quantity of organoids made by a method of generating kidney organoids, including providing a quantity of nephron progenitor cells (“NPCs”), and culturing the NPCs in a suspension culture for at about 11 days. In other embodiments, the method includes addition of one or more of: CHIR99021 and FGF9. In other embodiments, the kidney organoids comprise one or more cell types selected from: podocyte-like cells, proximal tubules, descending limbs of Henle, thick ascending limbs of Hendle, and distal convoluted tubules. In other embodiments, podocyte-like cells express one or more of: NPHS1+, PODXL+, and WT1+. In other embodiments, proximal tubules express one or more of: LTL+ and AQP1+. In other embodiments, descending limbs of Henle express one or more of: CDH1+ and AQP1+. In other embodiments, thick ascending limbs of Henle express one or more of CDH1+ and UMOD+. In other embodiments, distal convoluted tubules express one or more of CDH1+UMOD−. In other embodiments, NPCs are derived from human pluripotent stem cells (“hPSCs”). In other embodiments, hPSCs are derived from a patient suffering a disease mutation. In other embodiments, hPSCs have been genomically edited using CRISPR. In other embodiments, NPCs are derived from hPSCs by a method for generating metanephric mesenchyme, including providing a quantity of human pluripotent stem cells (“hPSCs”), generating late primitive streak cells, inducing formation of posterior intermediate mesoderm cells, and differentiating into metanephric mesenchyme cells. In other embodiments, the human pluripotent stem cells are human embryonic stem cells (“hESCs”). In other embodiments, the human pluripotent stem cells are human induced pluripotent stem cells (“hiPSCs”). In other embodiments, generating late primitive streak cells includes culturing hPSCs in CHIR99021 for about 3-5 days. In other embodiments, this includes about 4 days. In various embodiments, the concentration of CHIR99021 is about 3, 4, 5, 6, 7, 8, 9, 10, 11, 12, 13, 14, 15 μM. In various embodiments, the concentration of CHIR99021 is about 8-10 μM. In other embodiments, the method further includes addition of noggin In various embodiments, the concentration of noggin is about 1, 2, 3, 4, 5, 6, 7, 8, 9, 10 ng/ml. In various embodiments, the concentration of noggin is about 5 ng/ml. In other embodiments, inducing formation of posterior intermediate mesoderm cells includes culturing in the presence of activin for about 2-4 days. In other embodiments, this includes about 3 days. In various embodiments, the concentration of activin is about 5-10, 10-20-30 ng/ml. In various embodiments, the concentration of activin is about 10 ng/ml. In other embodiments, differentiating into metanephric mesenchyme cells includes addition of FGF9. In various embodiments, the concentration of FGF9 is about 5-10, 10-20-30 ng/ml. In various embodiments, the concentration of FGF9 is about 10 ng/ml. In other embodiments, the metanephric mesenchyme lineage cells are further differentiated into nephronic progenitor cells (NPCs) by addition of CHIR99021. In various embodiments, the concentration of CHIR99021 is about 1, 2, 3, 4, 5, 6, 7, 8, 9, 10 μM. In various embodiments, the concentration of CHIR99021 is about 5 μM. In other embodiments, late primitive streak cells express one or more of: T and TBX. In other embodiments, posterior intermediate mesoderm cells express one or more of: WT1 and HOXD11. In other embodiments, metanephric mesenchyme lineages cells express one or more of: SIX2, SALL1, WT1, and PAX2. In other embodiments, NPCs express one or more of: SIX2, SALL1, WT1, PAX2, and EYA1. In other embodiments, differentiation into metanephric mesenchyme cells is at least 50, 60, 70% or more efficient. In other embodiments, differentiation into metanephric mesenchyme cells is at least 70, 80, 90% or more efficient.

Also described herein is a method of screening a compound for an effect on kidney organoids, including providing a quantity of kidney organoids, adding one or more compounds to the kidney organoids, determining changes to phenotype or activity of the kidney organoids, and correlating the changes with an effect of the compounds on kidney organoids, thereby screening the one or more compounds for an effect on kidney organoids.

In various embodiments, determining changes to phenotype or activity includes detecting one or more markers in the tubular organoids.

### EXAMPLE 1

### Maintenance of hPSCs

H9 human ESCs (passage 45-65), and HDF-α human iPSCs (hiPSC derived from healthy fibroblasts; passage 22-42) were maintained in ReproFF2 (ReproCELL, #RCHEMD006) supplemented with FGF2 (10 ng/mL) (Peprotech, #100-18B) in 6-well tissue culture plates (Falcon, #353046) coated with 1% vol/vol LDEV-Free hESC-qualified Geltrex (Life Technologies, #A1413302) in a 37° C. incubator with 5% CO₂. hPSCs were passaged using Dissociation Solution for human ES/iPS cells (ReproCELL, #RCHETP002) at a 1:3 split ratio every 7 days according to the manufacturer's protocol. H9 was purchased from WiCell. HDF-α human iPSCs was previously established in the Inventors' laboratory.

### EXAMPLE 2

### Differentiation of hPSCs

hPSCs grown on Geltrex were washed once with PBS (Life Technologies, #10010-049) and dissociated into single cells with Accutase (STEMCELL Technologies, #07920). Cells were then plated at a density of 2-2.4×10⁴ (H9) or 1-1.4×10⁴ (HDF, 2C) cells/cm² onto 24-well tissue culture plates (TPP, #92024) coated with 1% Geltrex in ReproFF2 supplemented with the ROCK inhibitor Y27632 (10 μM) (TOCRIS, #1254) and FGF2 (10 ng/ml). After 72 hours, cells (50% confluent) were briefly washed in PBS and then cultured in basic differentiation medium consisting of Advanced RPMI 1640 (Life Technologies, #12633-020) and 1X L-GlutaMAX (Life Technologies, #35050-061) supplemented with CHIR99021 (8-10 μM) (TOCRIS, #4423) for 4 days to induce late primitive streak cells Noggin (5 ng/ml) was also used for hiPSC differentiation in addition to CHIR (10 μM). To induce posterior intermediate mesoderm, cells were then cultured in Advanced RPMI+1X L-GlutaMAX+activin (10 ng/mL) (R&D, #338-AC-050) for 3 days. For induction of nephron progenitor cells, the media was then changed to Advanced RPMI+1X L-GlutaMAX+FGF9 (10 ng/ml) (R&D, #273-F9-025/CF) for 7 days. CHIR (3 μM) was added to the media from day 9 to 11 of differentiation to induce renal vesicles. On day 14, cells were switched to the basic differentiation medium and cultured for an additional 7 to 14 days (total of 21 to 28 days). The medium was replaced every 2 or 3 days. A variety of growth factors and small molecules were tested for differentiation.

### EXAMPLE 5

### 3D Kidney Organoid Formation

hPSCs on day 9 of differentiation, which represents metanephric mesenchyme cells, were dissociated with Accutase and resuspended in the basic differentiation medium supplemented with CHIR (3 μM) and FGF9 (10 ng/mL), and placed in 96-well, round bottom, ultra-low attachment plates (Corning, #7007) at 1×10⁵ cells per well. The plates were centrifuged at 1500 rpm for 15 seconds, and the cells then cultured at 37° C., 5% CO₂ for 2 days. The medium was then changed to the basic differentiation medium supplemented with FGF9 10 ng/mL and cultured for 3 more days. After that, the organoids were cultured in basic differentiation medium with no additional factors for 7-21 days (a total of 21-35 days).

### EXAMPLE 4

### Nephrotoxicity Assay

3D kidney organoids were cultured in basic differentiation medium supplemented with gentamicin 5×10⁻⁴, 5×10⁻², or 5 mg/mL (Sigma, #G1264) for 48 hours or cisplatin 5 or 50 μM (Sigma, #P4394) for 2, 6, 24 or 48 hours after day 21 of differentiation. Organoids were then fixed with 4% paraformaldehyde (Electron Microscopy Sciences, #RT15710) for 20 minutes for both whole-mount and frozen section immunohistochemistry.

### EXAMPLE 5

### Immunocytochemistry

Cell cultures were washed once with PBS and fixed in 4% paraformaldehyde for 15 minutes at room temperature (RT). Fixed cells were washed three times in PBS and incubated in blocking buffer (0.3% Triton X-100 and 5% normal donkey serum) for 1 hour at RT. The cells were then incubated with primary antibody overnight at 4° C. or for 2 hours at RT in antibody dilution buffer (0.3% Triton X-100 and 1% BSA in PBS). Cells were then washed three times in PBS and incubated with Alexa Fluor 488-, 555-, or 647-conjugated secondary antibodies (1:500) (Life Technologies) in antibody dilution buffer for 1 hour at RT. For immunostaining with biotinylated LTL (Vector Labs, #B-1325), Streptavidin/Biotin Blocking Kit (Vector Labs, #SP-2002) and Alexa Fluor 488- or 647-conjugated streptavidin (Lafe Technologies) were used according to manufacturer's instructions. Nuclei were counterstained with DAPI (Sigma, #D8417). Immunofluorescence was visualized using an inverted fluorescence microscope (Nikon Eclipse Ti). Quantification was performed using ImageJ by counting three to five representative fields per experiment at 20× magnification. The sample number of biological replicates in each experiment is shown in figure legends.

### EXAMPLE 6

### Whole-Mount Immunohistochemistry of 3D Organoids

3D kidney organoids were fixed with 4% paraformaldehyde in PBS for 20 minutes at RT in a 96-well plate, then washed three times in PBS. The organoids were then incubated in blocking buffer (0.3% Triton X-100 and 5% normal donkey serum) for 1 hour at RT, then washed three times in PBS. The organoids were incubated with primary antibodies in antibody dilution buffer (0.3% Triton X-100 and 1% BSA in PBS) overnight at 4° C. The organoids were then washed with PBS three times for 1 hour each, with the third washing performed overnight at 4° C. For immunostaining with biotinylated LTL (Vector Labs, #B-1325), a Streptavidin/Biotin Blocking Kit (Vector Labs, #SP-2002) was used according to the manufacturer's protocol. The organoids were incubated with secondary antibodies in antibody dilution buffer for 1 hour at RT, then washed with PBS three times for 30 minutes each. Nuclei were counterstained with DAPI for more than 30 minutes. The organoids were then mounted with Vectashield (Vector Labs, #H-1200) and examined by confocal microscopy (Nikon Cl, Tokyo, Japan).

### EXAMPLE 7

### Immunohistochemistry of 3D Organoids

3D kidney organoids were fixed with 4% paraformaldehyde in PBS for 20 minutes in a 96-well plate, washed three times in PBS, then incubated with 30% sucrose (w/w) overnight at 4° C. The organoids were mounted with O.C.T compound (Fisher Scientific, #23-730-571) to make frozen blocks and were cut into 10-μm sections. The sections were washed three times in PBS for 5 minutes each, then incubated in blocking buffer (0.3% Triton X-100 and 5% normal donkey serum) for 1 hour. The sections were incubated with primary antibodies in antibody dilution buffer (0.3% Triton X-100 and 1% BSA in PBS) for 2 hours, then washed three times in PBS. The sections were incubated with secondary antibodies in antibody dilution buffer for 1 hour, then washed three times in PBS. The sections were then treated with Vectashield with DAPI. Imaging was performed with a Nikon Cl confocal microscope.

### EXAMPLE 8

### Quantitative RT-PCR

Total RNA was purified from cells using the RNAeasy MiniKit (Qiagen). 500 ng of RNA was used for reverse transcription with High-Capacity cDNA Reverse Transcription Kit (Life Technologies, #4368814) according to the manufacture's protocol. RT-PCR reactions were run in duplicate using cDNA (diluted 1:10), 300 nM forward and reverse primers, and iTAQ SYBR Green Supermix (Bio-Rad, #172-5122). Quantitative RT-PCR was performed using the iQ5 Multicolor Real-Time PCR Detection System (Bio-Rad). All samples were run with two technical replicates. β-actin was used as the housekeeping gene. Values were calculated by the delta delta CT method. Primer sequences are listed in Table 1.

TABLE 1
Primer Sequences

| Gene | | |
|---|---|---|
| Name | Forward | Reverse |
| ACTB | CTCTTCCAGCCTTCCTTCCT [SEQ ID | AGCACTGTGTTGGCGTACAG |
| | NO: 1] | [SEQ ID NO: 2] |
| BMP4 | AAGCGTAGCCCTAAGCATCA [SEQ | TGGTTGAGTTGAGGTGGTCA |
| | ID NO: 3] | [SEQ ID NO: 4] |
| KIMI | CGACAACGACTGTTCCAATG [SEQ | AAAGGCATTGGAGGAACAAA |
| | ID NO: 5] | [SEQ ID NO: 6] |
| MIXLI | ACGTCTTTCAGCGCCGAACAG [SEQ | TTGGTTCGGGCAGGCAGTTCA |
| | ID NO: 7] | [SEQ ID NO: 8] |
| OSRI | CCTTCCTTCAGGCAGTGAAC [SEQ | CGGCACTTTGGAGAAAGAAG |
| | ID NO: 9] | [SEQ ID NO: 10] |
| PAX2 | ACTCCATCAATGGGATCCTG [SEQ | CCACACCACTCTGGGAATCT |
| | ID NO: 11] | [SEQ ID NO: 12] |

### EXAMPLE 9

### Flow Cytometry

Cells were dissociated using Accutase for 10 minutes, and cell clumps were removed with a 40-μm cell strainer (Corning, #352340). Cells were fixed with 2% paraformaldehyde for 15 minutes on ice and then permeabilized with 0.1% Triton for 15 minutes on ice. Cells were then blocked with PBS+5% donkey serum for 15 minutes and incubated with primary antibodies (PAX8 1:2500, LHX1 1:100, SIX2 1:1000, SALL1 1:100, WT1 1:100) for 30 minutes. After washing three times with 1% BSA in PBS, cells were incubated with secondary antibodies (Alexa Fluor 488-conjugated donkey anti-rabbit 1:5000 [Life Technologies], Cy5-conjugated donkey anti-mouse 1:2500 [Jackson ImmunoResearch] or Alexa Fluor 647-conjugated donkey anti-mouse 1:5000 [Life Technologies]) for 20 minutes on ice. Cells were then washed three times with 1% BSA in PBS. Flow cytometry was performed using MACSQuant (Miltenyi Biotec). Optimal dilution ratios of antibodies were determined using negative controls, undifferentiated H9 and human proximal tubular cell line (HKC-8) that does not express PAX8, LHX1, SIX2, SALL1, or WT1. HKC-8 was kindly provided by Dr. Lorraine Racusen (Johns Hopkins Hospital).

### EXAMPLE 10

### Transmission Electron Microscopy

3D kidney organoids were fixed with 4% PFA for 20 minutes and subsequently fixed with electron microscopy (EM) fixation buffer consisting of 1.5% glutaraldehyde, 1% paraformaldehyde, 70 mM NaPO4 pH 7.2, and 3% sucrose in water overnight at 4° C. The organoids were washed three times in 0.2 M cacodylate buffer pH 7.4 for 10 minutes each and were incubated with 1% OsO4 for 1 hour on ice. The organoids were then washed three times in 0.2M cacodylate buffer pH 7.4 for 10 minutes each, dehydrated through a graded series of ethanol solutions, and embedded in Epon. 70 nm sections were cut and analyzed on a JEM-1010 (JEOL).

### EXAMPLE 11

### Cell Culture

HKC-8 was maintained in DMEM/F12 (Life Technologies, #11320-033) supplemented with 10% fetal bovine serum (FBS) in a 37° C. incubator with 5% CO₂, and was passaged every 3 or 4 days. NIH3T3-Wnt4 was maintained in DMEM (Corning, #10-013-CV) supplemented with 10% FBS in a 37° C. incubator with 5% CO₂, and was passaged every 3 or 4 days. NIH3T3-Wnt4 was kindly provided by Dr. Andrew P. McMahon. A mouse ureteric bud cell line was maintained in DMEM (Corning, #10-013-CV) supplemented with 10% FBS in a 37° C. incubator with 5% CO₂, and was passaged every 3 or 4 days. A mouse ureteric bud cell line was kindly provided by Dr. Jonathan Barasch. Mycoplasma contamination was tested by DAPI staining in all cell lines.

Recent efforts to direct the differentiation of PSCs into cells of the kidney lineage have focused the first step of differentiation on induction of the posterior primitive streak using a combination of growth factors that includes BMP4. The inclusion of BMP4 is justified by evidence that a gradient of Wnt3a and BMP4 patterns the anterior-posterior axis of the mouse primitive streak. However, recent developmental studies on early mesoderm patterning led us to reconsider this rationale. First, cells originating in the posterior primitive streak give rise to lateral plate mesoderm and not IM, from which the kidneys are derived (FIG. 8a). Second, the timing of migration of mesodermal precursors out of the primitive streak determines mesodermal patterning along the anterior-posterior axis. Thus, precursor cells of the more anterior mesoderm migrate out of the primitive streak earlier than those of posterior mesoderm. The Inventors therefore hypothesized that the embryonic origin of the posterior IM was not the cells of the posterior primitive streak but rather cells of the late-stage primitive streak, and that precisely recapitulating the developmental pathway defining both the anterior-posterior position along the primitive streak as well as the timing of migration out of the primitive streak would optimize the differentiation of PSCs into posterior IM.

To test this hypothesis, the Inventors treated human embryonic stem cells (hESCs; H9) with varying doses and durations of CHIR, which the Inventors and others previously showed could effectively differentiate hPSCs into T+primitive streak, solely or in combination with multiple developmental growth factors and small-molecule inhibitors of developmental signaling pathways (FIG. 1a, b). High dose CHIR (8 μM) over 4 days robustly induced and maintained a population of T+TBX6+ primitive streak cells (FIG. 1b-d, FIG. 8b, c, d). Subsequent treatment with activin (10 ng/mL) between days 4 and 7 successfully induced WT1+HOXD11+ cells with nearly 90% efficiency whereas WT1+HOXD11− cells were induced without activin (FIG. 1e-g, FIG. 8e). PAX2 and LHX1 were not expressed (FIG. 1f), confirming that activin treatment of late primitive streak cells produced posterior and not anterior IM cells. Moreover, shortening or extending CHIR treatment did not efficiently induce WT1+HOXD11+ cells after subsequent activin treatment, indicating that 4 days treatment of CHIR was optimal for efficient induction of late primitive streak and then posterior IM.

### EXAMPLE 12

### Differentiation of hPSCs into Posterior Intermediate Mesoderm

To confirm the reproducibility of posterior IM induction in other hPSC lines, the Inventors next tested the combination of high-dose CHIR with activin, BMP4, FGF2, FGF8, FGF9, IDE-1, JAG1, Noggin or Y-27632 for 4 days followed by treatment with activin in HDF-α human induced pluripotent stem cells (hiPSCs). Intrinsic differences between HDF-α hiPSCs and H9 ESCs mandated slight modifications to the protocol to optimize the production of posterior IM cells. HDF-α hiPSCs required a higher dose of CHIR (10 μM) to induce T+TBX6+ primitive streak with an efficiency similar to that of H9 hESCs (FIG. 9a). When HDF-α hiPSCs treated with CHIR 10 μM for 4 days were then treated with activin for 3 days, HOXD11, but not WT1, was expressed on day 7 (FIG. 9e). The absence of WT1 suggested a failure of these factors to induce posterior IM in hiPSCs. To determine whether other posterior mesoderm subtypes had been induced by the Inventors' differentiation protocol, the Inventors immunostained H9 hESCs and HDF-α hiPSCs on day 4 following treatment with CHIR. The hiPSCs but not the hESCs expressed FOXF1, a marker of the posterior primitive streak and lateral plate mesoderm (FIG. 9b).

As the posterior primitive streak is induced by a BMP4 signal gradient, the Inventors hypothesized that CHIR treatment of HDF-α hiPSCs might induce endogenous BMP4 production that promotes differentiation into the posterior primitive streak and subsequently lateral plate mesoderm. The addition of low dose noggin (5 ng/mL), a BMP4 signaling antagonist, with CHIR in the first step of differentiation suppressed BMP4 to an optimal level to yield cells that expressed T and TBX6 but not FOXF1 on day 4 (FIG. 9c). Subsequent differentiation with activin resulted in the generation of WT1+HOXD11+ posterior IM cells with 80-90% efficiency (FIG. 1b-g, FIG. 9e). Although H9 hESCs did not express FOXF1 on day 4 in response to CHIR, the addition of low concentrations of BMP4 (1 ng/mL) resulted in FOXF1 expression at day 4 and suppression of WT1 expression on day 7, similar to what the Inventors observed in the hiPSC line (FIG. 9d). Collectively, these results demonstrated that the efficacy of posterior IM induction was highly sensitive to the presence of BMP4 signaling at the primitive streak stage in hPSCs. Optimizing the protocol for individual cell lines based on their endogenous BMP4 production maximized the efficiency of generating posterior IM.

### EXAMPLE 13

### Induction of Multipotent Nephron Progenitor Cells

To differentiate WT1+HOXD11+ posterior IM cells into NPCs of the metanephric mesenchyme, the Inventors treated them on day 7 with varying doses of FGF9 (5-200 ng/mL), which the Inventors and others have previously shown to induce SIX2+ cells. A low dose of FGF9 (10 ng/mL) was sufficient to induce SIX2+ cells with an efficiency of 90% within 1-2 days of treatment (FIG. 2a, b). These SIX2+ cells co-expressed other critical markers of the NPCs of the metanephric mesenchyme, including SALL1, WT1, PAX2, and EYA1 (FIG. 2b-d), as assessed by immunocytochemistry and flow cytometry. The Inventors therefore considered these SIX2+SALL1+WT1+PAX2+ cells to be putative NPCs. Though the Inventors were unable to assay OSR1 expression by immunostaining due to the lack of highly specific antibodies, the Inventors could detect high levels of OSR1 transcript as early as day 7 (posterior IM) and sustained through day 9 (NPCs) by quantitative real-time PCR (FIG. 2e). SIX2 expression in these cells could be sustained for at least 1 week with continuous exposure to FGF9 (FIG. 2f, g).

### EXAMPLE 14

### Differentiation into Nephron Progenitor Cells and Spontaneous Formation of Renal Vesicles

Even with continuous FGF9 treatment, the Inventors observed between days 10 and 14 of differentiation that some of the NPCs spontaneously downregulated SIX2 expression and differentiated into round, polarized clusters of PAX8+LHX1+ cells reminiscent of renal vesicles (FIG. 2f, g, FIG. 10a). Upon withdrawal of FGF9 on day 10, these renal vesicle-like clusters proceeded to expand and elongate into tubular structures, resembling the process of nephron formation in vivo (FIG. 10b). Immunocytochemistry of the elongated structures revealed that they contained Lotus tetragonolobus lectin (LTL)+N-cadherin (CDH2)+ and Nephrin (NPHS1)+Podocalyxin (PODXL)+ cells reminiscent of proximal tubules and podocytes, respectively (FIG. 10c). Thus, in the absence of exogenous signals after day 10, hPSC-derived NPCs are intrinsically programmed to differentiate into early-stage epithelial structures of the nephron.

To test whether the formation of epithelial structures could be facilitated by 3D culture conditions, on day 10, at the time of FGF9 withdrawal, the Inventors replated NPCs in ultra-low-attachment, round-bottom 96-well plates and cultured them for 1 week. NPCs formed 3D spherical aggregates, one per 96-well, in suspension culture. Whole-mount staining of aggregates at day 16 revealed the presence of LTL+tubules and clusters of NPHS1+PODXL+WT1+ cells (FIG. 10g, h). Co-culturing NPCs in 3D with Wnt4-expressing NIH3T3 mouse embryonic fibroblast cells or a ureteric bud cell line did not significantly increase the LTL+ tubules in organoids (FIG. 10d-f), in contrast to a previous report. Similarly, the addition of six growth factors (BMP7, HGF, IGF-1, JAG1, WNT4 and mouse Wnt9b) or three small molecules (CHIR, IWR-1, SB431542), did not significantly increase the number of LTL+ tubules (FIG. 10e). These results suggested the need for more accurate recreation of temporal signal activation.

### EXAMPLE 15

### Spontaneous Morphogenesis into Nephron Structures

Although the Inventors observed spontaneous differentiation of SIX2+ NPCs into PAX8+LHX1+ renal vesicles and early nephron epithelia in both 2D and 3D culture, the efficiency of this process was relatively low. During mouse nephrogenesis, Wnt9b, secreted by the ureteric bud, induces the metanephric cap mesenchyme to undergo a mesenchymal to epithelial transition via upregulation of Wnt4 in nephron progenitors, and the inductive Wnt signals are subsequently suppressed during formation of renal vesicles, possibly by Dkk1. This induction can be mimicked ex vivo or in vitro by the transient treatment of isolated mouse metanephric mesenchyme or FACS-sorted SIX2+ cells with the GSK-3β inhibitor BIO. The Inventors therefore sought to improve the efficiency of generating renal vesicles through transient rather than sustained activation of Wnt signaling in NPCs. Through a systematic screen of growth factors and small molecules, including CHIR, (FIG. 11a, b) in 2D cultures, the Inventors found that treating NPCs with low dose CHIR (3 μM) for 2 days from day 9 while simultaneously maintaining exogenous FGF9 signaling (days 7-14) markedly increased the number of cells that downregulated SIX2 and co-expressed PAX8 and LHX1 (FIG. 3a-c, FIG. 11c). Quantification by flow cytometry revealed that ˜75.9% of differentiated cells were PAX8+LHX1+ (FIG. 3d). PAX8+LHX1+ cells were organized in laminin-bound, polarized round clusters morphologically resembling renal vesicles in vivo (FIG. 3e). The cells also expressed HNF1β and BRN1, the other makers of renal vesicles, suggesting that they represented renal vesicles (FIG. 3e). In 3D culture from day 9, the same treatment with CHIR and FGF9 induced similar structures: PAX8+LHX1+LAM− pre-tubular aggregates and PAX8+LHX1+LAM+ renal vesicles became apparent on day 11 and 14, respectively, and grew larger over time (FIG. 3f, g).

### EXAMPLE 16

### Induction of Pre-Tubular Aggregates and Renal Vesicles from Nephron Progenitor Cells

By day 21 of differentiation, the renal vesicles spontaneously formed elongated epithelial nephron structures without additional factors (FIG. 4a-c). These structures expressed segmental markers of the nephron in a contiguous arrangement, including glomerular podocytes (NPHS1+PODXL+), proximal tubules (LTL+CDH2+), and loop of Henle/distal tubules (E-cadherin (CDH1)+Uromodulin (UMOD)+BRN1+) (FIG. 4c-e). Nephron-like structures were generated by day 21 of differentiation, with an efficiency>20 times greater than that of the Inventors' previous protocol (FIG. 4f)²⁰, and could be sustained until at least day 56. No outgrowth of collecting duct (CDH1+Dolichos biflorus agglutinin (DBA)+) structures, which are derived from the ureteric bud and not metanephric mesenchyme, from renal vesicles was seen, confirming that the Inventors' differentiation protocol was specific for producing NPCs of the metanephric mesenchyme.

### EXAMPLE 17

### Self-Organizing Nephron Formation in 2D Culture Generation of In Vitro Kidney Organoids in 3D Culture

Next the Inventors investigated whether a 3D culture environment could promote the formation of more organized nephron structures with tubules possessing a lumen. The Inventors replated cells cultured in 2D on days 9, 11 and 14 corresponding to NPCs, pre-tubular aggregates and renal vesicles, respectively, into 3D suspension culture and applied the same protocol as with 2D (FIG. 4a). Re-plating day 9 cultures showed the greatest induction of nephron-like structures. Whole-mount immunostaining of organoids from days 21-35 of differentiation revealed numerous contiguous nephron-like structures with features of nephron segments from glomerulus to distal tubule (FIG. 5a-c). Clusters of podocyte-like cells (NPHS1+PODXL+WT1+) were surrounded by Bowman's capsule-like structures, connected to tubular structures with markers of proximal tubules (LTL+AQP1+), descending limbs of Henle (CDH1+AQP1+), thick ascending limbs of Henle (CDH1+UMOD+), and distal convoluted tubules (CDH1+UMOD−) expressed in the same sequence as in the in vivo nephron (FIG. 5d). SIX2 expression was absent in 3D kidney organoids, suggesting that NPCs had completely differentiated into nephron epithelia. Furthermore, the expression of SALL1, another NPC marker that is transiently expressed in immature nephron tubules, was not detected in nephron-like structures, indicating that these structures were more mature than those in prior reports. Characterization of the non-epithelial cells comprising the interstitial compartment between nephron-like structures revealed that these cells were negative for the metanephric stromal progenitor marker FOXD1, the endothelial marker Endomucin, and the fibroblast marker α-Smooth muscle actin, indicating that these cell types did not constitute a significant cell population within the organoids (data not shown).

### EXAMPLE 18

### Self-Organizing Nephron Formation in 3D Culture

Electron microscopy of the kidney organoids at day 21 of differentiation revealed ultrastructural features characteristic of mature renal epithelia. Structures resembling foot processes were noted on the surface of podocyte-like cells, which were encapsulated by a layer of cells reminiscent of Bowman's capsule (FIG. 5e,f, upper panels). Tubular structures possessed a discrete lumen and epithelial tight-junctions similar to kidney tubules, and a subset of the tubules comprised mitochondria-rich cells with brush border-like structures, characteristic features of proximal tubular cells (FIG. 5e,f, lower panels). These findings demonstrate that differentiation of the NPCs in suspension culture results in the formation of 3D kidney organoids and organized, multi-component nephron-like structures with distinct lumens in a contiguous and sequential arrangement that mimics the nephron.

### EXAMPLE 19

### Modeling Kidney Development and Toxicity with Organoids

The observation that no additional exogenous chemicals are required for hPSC-derived renal vesicles to form more mature nephron structures indicated that the signals for nephron formation are endogenously activated after renal vesicles are formed. As patterning of the nephron into its different segments begins at the renal vesicle stage during development, the Inventors postulated that developmental patterning could be mimicked by chemical modulation of these endogenous signals. Addition of the Notch signaling inhibitor DAPT from day 14 to 21 of differentiation resulted in a marked suppression of proximal tubule formation in both 2D and 3D culture (FIG. 6a, b, FIG. 12a-c), consistent with previous studies implicating Notch signaling in proximal patterning of the nephron.

### EXAMPLE 20

### Modeling Kidney Development and Injury in Kidney Organoids

Drug nephrotoxicity is an important cause of acute kidney injury in hospitalized patients. Currently there are no patient-specific models to assay nephrotoxicity in vitro. To test whether the Inventors' organoids could be used to study kidney injury and toxicity in vitro, the Inventors treated 3D hESC-derived kidney organoids after 21 days of differentiation for 48 hours with gentamicin (5 mg/mL), a commonly used antibiotic with well-established proximal tubular toxicity, or for 24 hours with cisplatin (5 an anticancer drug with proximal and distal tubular toxicity. Organoids were then fixed and immunostained for Kidney Injury Molecule-1 (KIM-1), a biomarker that is highly upregulated in the proximal tubules following acute kidney injury, together with LTL and E-cadherin to identify proximal and distal tubules, respectively.

Staining of both whole mount and frozen sections of gentamicin-treated organoids demonstrated clear KIM-1 expression at the luminal surface of LTL+tubules but not in E-cadherin+ tubules, and real-time PCR showed KIM-1 upregulation by gentamicin in a dose-dependent manner (FIG. 6c, d, e, FIG. 13a, b), indicating that gentamicin had injured proximal tubules in the organoids. Moreover, cisplatin significantly upregulated KIM-1 in LTL+ tubules and suppressed E-cadherin expression (FIG. 6c), indicating proximal and distal tubular toxicity. No KIM-1 expression was observed in either LTL+ or E-cadherin+ tubules in untreated organoids. To distinguish between a generalized toxic effect and nephron segment-specific injury, the organoids were immunostained for γH2AX, a marker of DNA damage (FIG. 6f). Cisplatin at a dose of 5 μM upregulated γH2AX expression in LTL+ tubules but not in PODXL+ podocytes, whereas a higher dose of cisplatin (50 μM) resulted in more widespread γH2AX expression, consistent with more generalized cell toxicity. These findings establish the utility of the Inventors' 3D kidney organoid system as a patient-specific model of toxic kidney injury that can be employed to test the nephrotoxicity of drugs and other chemicals in vitro.

### EXAMPLE 21

### Discussion

The Inventors describe the generation of segmentally patterned nephron structures from hPSCs by directed differentiation. The Inventors' protocol efficiently induces NPCs that spontaneously form renal vesicles in both 2D and 3D culture, which subsequently differentiate into self-organized nephron-like structures containing glomeruli, proximal tubules, loops of Henle, and distal tubules in a contiguous, ordered arrangement analogous to that of nephrons. To the Inventors' knowledge, no previous study has converted hPSCs into nephron structures with mature contiguous, ordered segments. Ref 18 generated SIX2+ cells, with an efficiency of 20%, that formed 3D aggregates containing isolated tubular structures but not continuous nephron structures with all epithelial components, and the protocol generated cells of both the metanephric mesenchyme and ureteric bud lineages, suggesting a lack of specificity. In comparison, the Inventors' method generates SIX2+SALL1+WT1+PAX2+ NPCs with 90% efficiency, and the cells spontaneously give rise to nephron structures containing all the major epithelial derivatives of the metanephric mesenchyme without detectable ureteric bud derivatives. Induction of SIX2+ cells from posterior IM in the Inventors' protocol required very low doses of FGF9 (10 ng/mL) compared to a concentration 20 times higher in ref. 18, suggesting that the WT1+HOXD11+ posterior IM cells are primed to respond to FGF9 and differentiate into NPCs. Taguchi and colleagues introduced the concept that targeting axial stem cells and posterior IM could facilitate the derivation of NPCs of the metanephric mesenchyme. Their protocol, based on an embryoid body culture system, required more intermediate steps and growth factors at each step and used mouse embryonic spinal cord to induce NPCs to undergo tubulogenesis. In addition, the persistent expression of SALL1 in the tubular structures indicated that they were still at an immature stage of nephron development. The Inventors' protocol to generate posterior IM and NPCs uses 2D monolayer culture, fewer steps, fewer chemicals and is fully chemically defined and more rapid. A key difference between the Inventors' protocol and previous ones is the Inventors' strategy to induce late-stage mid primitive streak rather than posterior primitive streak, based on developmental studies showing that the posterior primitive streak gives rise to lateral plate mesoderm rather than IM. By precisely defining the appropriate anterior-posterior position within the primitive streak (with the dose of CHIR and suppression of BMP4) and the timing of cell migration out of the primitive streak (with the duration of CHIR treatment), the Inventors could generate the correct precursor population that would give rise to NPCs. As predicted, posteriorization of the primitive streak with the addition of BMP4 causes hPSCs to differentiate into FOXF1+ lateral plate mesoderm. Finally, the Inventors show that minor modifications in the protocol optimize the efficiency of directed differentiation in both hESC and hiPSC lines. Variability in the levels of endogenous BMP4 signaling markedly affected the Inventors' ability to differentiate an hiPSC line into posterior IM, but this could be addressed by adjusting BMP4 levels with the addition of the antagonist Noggin.

As shown in previous studies of differentiating hPSCs to other lineages, the Inventors find that closely recapitulating critical developmental stages in vitro improves differentiation efficiencies and produces cells that most closely resemble their in vivo counterparts. The Inventors' hPSC-derived NPCs express all of the markers of metanephric mesenchyme and possess the intrinsic ability to spontaneously differentiate into renal vesicles and nephrons. Although the absence of ureteric bud and vascular progenitors in the Inventors' system precludes the generation of collecting ducts and glomerular capillaries, respectively, the NPC-derived renal vesicles self-organize into nephrons without these components in both 2D and 3D contexts.

The ability to generate 3D kidney organoids containing self-organized nephrons will facilitate studies of kidney development, disease and injury and of cell replacement therapies. Similar organoid systems have shown promising results for modeling the brain and gastric symptoms. The Inventors' data demonstrating that Notch inhibition suppresses proximal tubular differentiation confirms the utility of the Inventors' system for studying mechanisms of human kidney development, for which no models currently exist. Using gentamicin and cisplatin, the Inventors have also shown how the presence of the major epithelial components of the nephron in the organoids allows screening for toxic drug effects on multiple nephron segments. Given the individual variation in drug sensitivity in humans, the generation of kidney organoids from human iPSCs would enable drug testing in a patient-specific manner.

### EXAMPLE 22

### Maintenance of hPSCs in Feeder-Free Culture with ReproFF2

All maintenance culture experiments described here use ReproFF2 and 6-well plates coated with 1% LDEV-Free hESC-qualified Geltrex.
1| Before passaging hPSCs, check the cell conditions and extent of spontaneous differentiation. Healthy undifferentiated hPSCs display round colony morphology with a clear boundary. Proceed to the next step when the colonies are ˜80% confluent. Unlike when grown on feeder culture, it is usual to observe some merged colonies.

If differentiated cells are observed, remove those cells by aspiration. Those differentiated cells are usually located at the center of the colony when the size of each colony becomes too large. Some differentiated cells, however, sometimes exhibit fibroblast-like morphology at the periphery of the colonies. In that case, it is difficult to remove those differentiated cells; therefore, it is better to retry transition from “on feeder” to “feeder-free” from the beginning.
2| Aspirate differentiated colonies by visual recognition. Small differentiated colonies will spontaneously disappear upon passaging; therefore, it is sufficient to remove only large differentiated colonies. Aspirate ReproFF2 and add 200 μl of Dissociation Solution for human ES/iPS cells (CTK solution) from the center of the well in order to distribute the solution to the entire surface area. Incubate the cells in an incubator at 37° C. for 2 min until the edges of the colonies start to roll up. Aspirate CTK solution and add 2 ml of PBS to wash away the remnant CTK solution.

It is important to be careful with treatment with CTK solution which can be detrimental if exposed to cells too long. If one wait too long, colonies will be lost when subsequently washed with PBS. Hence, it is important to perform a visual check, with or without a microscope, for cell detachment after 2 min of exposure to CTK. When one notice that periphery of colonies starting to roll up, it is time to proceed to the next step. The Inventors recommend checking cells every 15 sec after 2 min of incubation with CTK solution.
3| Aspirate PBS and add 1 ml of ReproFF2. Detach colonies using a cell scraper. Break up colonies with a 1 ml pipette until one cannot visualize large aggregates. Pipetting is usually performed 3˜10 times (depends on confluency and cell lines).
The optimal size of colonies depends on the cell lines, the confluency, and the passage number in feeder-free culture. The optimal diameter of colonies is 100˜200 μm when the cells are optimized to feeder-free culture.
4| Prepare a new plate at room temperature (20-25° C.) for 30 min. Aspirate Geltrex solution from the new well, and add 1˜1.7 ml of ReproFF2 depending on the passage ratio (total will be 2 ml).
5| Transfer 330 μl˜1 ml (1:1˜1:3 ratio) of the colony fragments into 1 well of the new plate and shake the plates gently in order to distribute the colonies equally in the well.
6| Maintain the cells at 37° C. in a 5% CO₂ incubator. Replace the medium after 3 days and 5 days. Passage the cells every 7 days.

### EXAMPLE 23

### Feeder-Free hPSC Culture in ReproFF2 Medium (Step 1-6)

The Inventors' protocols use feeder-free hPSC culture in ReproFF2 medium with lactose dehydrogenase elevating virus (LDEV)-Free hESC-qualified Geltrex-coated plates. The Inventors maintain hPSCs in 6-well plates coated with 1% LDEV-Free hESC-qualified Geltrex with ReproFF2 medium, supplemented with fibroblast growth factor 2 (FGF2), 10 ng/ml (step 1-6). If hPSCs were initially cultured on mouse embryonic fibroblast (MEF) feeders, the Inventors recommend passaging the cells at least 5 times under feeder-free conditions with ReproFF2. If it is too difficult to maintain hPSCs in ReproFF2, the Inventors recommend using StemFit Basic supplemented with FGF2 (10 ng/ml) maintenance of hPSCs in 6-well plates coated with 1% LDEV-Free hESC-qualified Geltrex (Box 1). hPSCs are passaged every 7 days whether ReproFF2 or StemFit Basic is used.

### EXAMPLE 24

### Preparation of hPSCs for Differentiation TIMING 3 d

All differentiation experiments use 24-well plates coated with 1% LDEV-Free hESC-qualified Geltrex.
7| Check the confluency and spontaneous differentiation of the cells on the day of passaging. Usually, there are very few differentiated cells; therefore, it is not necessary to remove differentiated cells when one prepare the cells for differentiation. If differentiated cells are observed, aspirate the differentiated cells and make colonies smaller in subsequent passages.

If the confluency is not high enough, passage the cells at 1:1 ratio until the confluency is nearly 80%. Less confluency will result in poor viability of cells and inefficient differentiation once initiated.
8| Aspirate ReproFF2 and add 2 ml of PBS. Gently swirl the plate to wash out the remnant of ReproFF2. Aspirate PBS and add 500 μl of Accutase. Incubate the cells in an incubator at 37° C. and 5% CO₂ for 10 min. Tap the plate to facilitate detachment of cells. Incubate for another 5 min.
9| After incubation for 15 min in total, detach and dissociate the cells with a 1 ml pipette gently until one cannot recognize cell aggregates. Prepare 15 ml tubes by adding 500 μl ReproFF2.
10| Collect the dissociated cells into 15 ml tubes filled with 500 μl ReproFF2. Take 20 μl from 15 ml tubes to a Cellometer Counting Chamber for cell counting.
11| Centrifuge tubes at 300×g at room temperature for 4 min. While centrifuging tubes, count the cell number with a Cellometer. Aspirate the medium and resuspend cells at 10,000 cells/μl in ReproFF2.

When one collect cells from 1 well of 6-well plates, usually one have 2˜5 million cells. If one have fewer than 1 million cells, the cell confluency in the maintenance hPSC culture was too low, which will result in poor viability of cells when differentiation is initiated.
12| Aspirate the supernatant and resuspend the cells at 10,000 cells/μl in ReproFF2. Prepare sufficient amount of ReproFF2 with 10 μM of ROCK inhibitor Y27632 (1:1000 dilution) for the differentiation experiments. Transfer the cell suspension to ReproFF2 with Y27632 in order to have 44,000˜86,000 cells/ml (12,000˜24,000 cells/cm²). Plate cells in 500 μl/well of 24-well plates.
The plating density is critically important to achieve high efficiency of differentiation. It is vital to test different plating densities when one use different cell lines. For H9 cells, approximately 20,000 cells/cm² was best in the Inventors' experience. For HDF-α iPS cells, approximately 14,000 cells/cm² was optimal. In addition, the size of the well is also very important. If one want to change the size, it is necessary to test different plating densities.
13| Culture the cells at 37° C. in a 5% CO₂ incubator for 72 hours. There is no need to change the medium.
If one want to start the differentiation earlier, one need to plate more cells at step 12 to have nearly 50% confluent cells when one start the differentiation.

### EXAMPLE 25

### Preparation of hPSCs for Differentiation (Step 7-13)

The cells are plated for differentiation when the cells are passaged (step 7-13). The Inventors usually prepare 2 wells of 6-well plates, and use one well for differentiation and one well for continued passaging. Plating density significantly affects the differentiation efficiency, and each line of hPSCs requires adjustment. Pluripotency of hPSCs needs to be well maintained in the undifferentiated cells, and hence differentiated colonies need to be removed by aspiration. For differentiation, the cells are dissociated with Accutase for 15 min, resuspended in ReproFF2 supplemented with FGF2 10 ng/ml and Y27632 10 μM, and plated onto 24-well plates pre-coated with 1% LDEV-Free hESC-qualified Geltrex. The cells are cultured for 72 hours until the cells reach approximately 50% confluency.

### EXAMPLE 26

### Differentiation of hPSCs into Posterior Intermediate Mesoderm Cells TIMING 6˜7 d

14| Check the cell confluency. About 50% is the best to start the differentiation. Aspirate ReproFF2 and add 500 μl PBS to wash out the remnant of ReproFF2.
The confluency when differentiation is started affects cell viability and differentiation efficiency. If the confluency is less than 50%, one can adjust the timing to start the differentiation by waiting for an additional several hours.
15| Aspirate PBS and add 500 μl of the differentiation basal medium supplemented with CHIR 3˜10 μM+/−a BMP4 inhibitor (noggin 5-25 ng/ml, or dorsomorphin 100-500 nM).

The concentration of CHIR and addition of a BMP4 inhibitor depends on the cell line, the passaging number, and maintenance culture conditions. For H9, 8 μM of CHIR was best with ReproFF2 culture. For HDF, 10 μM of CHIR with 5 ng/ml noggin was best. If one use other cell lines or other culture media, adjust the protocol as follows: First, adjust the plating cell number to obtain 50% confluency when differentiation is initiated. Second, find the highest concentration of CHIR (3˜10 μM) which does not lead to cell detachment and death during 4 days of CHIR treatment. Third, test addition of a BMP4 inhibitor (noggin 5˜25 ng/ml or dorsomorphin 100˜500 nM), if the adjustment of the plating cell number and CHIR was not sufficient to induce SIX2+ cells.
16| Culture the cells at 37° C. in a 5% CO₂ incubator for 2 days.
17| Aspirate the differentiation medium and feed cells with 500 μl of the fresh differentiation medium supplemented with the same concentration of CHIR (with or without a BMP4 inhibitor).
18| Culture the cells at 37° C. in a 5% CO₂ incubator for 2 days.
19| Check the morphology of cells. The presence of loosely formed dense clusters of cells indicates the best time to switch to the next differentiation treatment (FIG. 15). Usually, 96 h of differentiation is the best timing; however, one can adjust the timing depending on the morphology of cells. Aspirate the medium and add 750 μl of the differentiation medium with activin A 10 ng/ml.

This stage is important to achieve high efficiency of differentiation to NPCs. Check the morphology very carefully. If the cells still form a homogenous flat monolayer (too loose, FIG. 15), feed cells with the differentiation medium with the same concentration of CHIR (with or without a BMP4 inhibitor) and wait for one-half to one full day. If the cells form dome-like round clusters (too dense, FIG. 15), similar to undifferentiated mouse embryonic stem cells, it is often too late to proceed to the next step. Try again from the beginning of differentiation by lowering the concentration of CHIR.
20| Culture the cells at 37° C. in a 5% CO₂ incubator for 2˜3 days. There is no need to feed cells. If the SIX2+cell induction was not good enough, test 2 days treatment of activin A instead of 3 days.

### EXAMPLE 27

### Differentiation of hPSCs into NPCs TIMING 1˜2 d

21| Aspirate the medium and add 500 μl of the basic differentiation medium supplemented with FGF9, 10 ng/ml.
22| Culture the cells at 37° C. in a 5% CO₂ incubator for 1˜2 days. One usually don't need to feed cells, but feed cells with the basic differentiation medium supplemented with FGF9, 10 ng/ml, if the medium becomes yellow in color.

### EXAMPLE 28

### Nephron Progenitor Cell Induction (step 14-22)

FIG. 15 shows the cellular morphology upon initiation of differentiation. The confluency at initiation significantly affects the differentiation efficiency; therefore, the Inventors strongly recommend the preparation of different plating densities until one find the best condition. First, the cells are briefly washed with PBS once in order to remove the remnant of ReproFF2 (or StemFit, if one choose to use this). Then, differentiation is initiated with CHIR99021 (CHIR) 3˜10 μM+/−a BMP4 inhibitor (noggin 5˜25 ng/ml or dorsomorphin 100˜500 nM). Each line of hPSCs requires a dose adjustment of CHIR. The highest dose which does not lead to cell detachment or death during 4 days of CHIR treatment is recommended. The addition of a BMP4 inhibitor depends on the cell line and the maintenance conditions that one use. When the Inventors use H9 cells maintained in ReproFF2, the Inventors do not require the addition of a BMP4 inhibitor to CHIR 8 μM. This first step of differentiation generally takes 4 days. The medium should be changed on day 2 of the differentiation. On day 4 of differentiation, the cells form loosely dense clusters (FIG. 15). This identifies the best time to proceed to the next step of differentiation, which involves treating the cells with activin A at 10 ng/ml.

After 3 days of activin A treatment, the markers for posterior intermediate mesoderm, namely WT1 and HOXD11, become positive. Then, the cells are treated with FGF9, 10 ng/ml, for 2 days to induce NPCs. On day 9 of differentiation, a critical marker for NPCs, SIX2, becomes positive. SIX2 staining is very bright when the differentiation is induced appropriately (FIG. 16a).

### EXAMPLE 29

### Differentiation of hPSCs into Kidney Organoids (2 Dimensional)˜TIMING 12 d

23| Aspirate the medium and add 500 μl-1 ml of the differentiation medium with FGF9 10 ng/ml and CHIR 3 μM.

Check the morphology of cells. If many cells form round polarized structures with lumens resembling renal vesicles (FIG. 15), one should adjust the treatment time of this step by shortening from 2 days to 1 day. If the medium color quickly changes to yellow, one should increase the volume of the medium up to 1 ml in the next experiments.
24| Culture the cells at 37° C. in a 5% CO₂ incubator for 2 days. If the medium color becomes yellow after 1 d of culture, feed the cells with the differentiation medium using the same concentration of CHIR and FGF9 with the same volume as in step 23.
25| Aspirate the medium and add 750 μl˜1 ml of the differentiation medium with FGF9, 10 ng/ml.
26| Culture the cells at 37° C. in a 5% CO₂ incubator for 2˜3 d.
If the medium color becomes yellow after 1 or 2 d of culture, feed the cells with the differentiation medium, using the same concentration of FGF9 with the same volume as in step 25. If round polarized structures with lumens, resembling renal vesicles, are observed, one can proceed to the next step. If renal vesicle structures are not observed within 3 days, one should confirm whether LHX1 is expressed by immunostaining. If LHX1 is positive in most of cells, one can proceed to the next step.
27| Aspirate the medium and add 500 μl of the basic differentiation medium without any growth factors.
28| Feed cells every 2˜3 days with 500˜750 μl of the basic differentiation medium. The kidney organoids are stable for at least up to 3 months of differentiation. Nephron structures can be recognized with a microscope (FIG. 15, FIG. 16e).

### EXAMPLE 30

### Differentiation of hPSCs into 3 Dimensional Kidney Organoids TIMING 12˜d

29| At step 22 when NPCs are induced, switch to 3 dimensional culture. Aspirate the medium and add 500 μl PBS. Wash out the remnant of the differentiation medium.
30| Aspirate PBS and add 300 μl Accutase to 1 well of 24-well plates. Incubate the cells at 37° C. in a 5% CO₂ incubator for 10˜15 min. Prepare 15 ml tubes containing 300 μl of the basic differentiation medium supplemented with CHIR 3 μM and FGF9 10 ng/ml.
31| Dissociate the cells with a 1 ml pipette, and transfer the cells to 15 ml tubes prepared at step 30. Take 20 μl from 15 ml tubes for cell counting with a Cellometer.
32| Centrifuge tubes at 300 g at room temperature for 4 min. Count the cell number using a Cellometer.
33| Aspirate the medium and resuspend the cells in the basic differentiation medium supplemented with CHIR 3 μM and FGF9 10 ng/ml at 500,000 cells/ml. Plate 100,000 cells/well in 200 μl of the differentiation medium onto 96-well, round bottom, ultra-low-attachment plates.
34| Centrifuge the plates at 300×g for 15 s, and culture the cells at 37° C. in a 5% CO₂ incubator for 2 days.
35| Gently aspirate the medium with a 200 μl pipette and add 200 μl of the basic differentiation medium supplemented with FGF9 10 ng/ml.
36| Culture the cells at 37° C. in a 5% CO₂ incubator for 2˜3 days until renal vesicle-like round structures become visible by a microscope.
37| Aspirate the medium with a 200 μl pipette, and add the basic differentiation medium without any additional growth factors.
38| Culture the cells at 37° C. in a 5% CO₂ incubator for at least for 1 week. Aspirate 85 μl of the medium and add 100 μl of the basic differentiation medium every 2-4 days.
39| Aspirate the medium and add 200 μl of 4% PFA. Incubate the plate at room temperature for 15˜30 min.

### EXAMPLE 31

### Kidney Organoid Induction (Step 23-39)

From this nephron progenitor cell stage, the Inventors can apply the same differentiation treatment in either 2D or 3D culture. When the Inventors switch to 3D culture, the Inventors use 96-well, round bottom, ultra-low attachment plates, and plate 100,000 cells/well. Usually, the Inventors obtain 2˜3 million cells from one well of 24-well plates, which is sufficient to generate many organoids. In both 2D and 3D culture, the Inventors treat NPCs with CHIR 3 μM and FGF9 10 ng/ml for 2 days in order to induce pre-tubular aggregates (PAX8+LHX1+). Then, the Inventors switch back to FGF9, 10 ng/ml alone, and culture the cells for 3 days to differentiate them into renal vesicles (PAX8+LHX1+LAM+). After that, the Inventors use only the basic differentiation medium without any growth factors, and the cells form segmented-nephron structures within one week. The kidney organoids generated by the Inventors' protocols are stable in the basic differentiation medium for up to 3 months with feeding every 2˜3 days.
40| Aspirate the 4% PFA and add 500 μl PBS. Wash the well gently and aspirate PBS.
41| Repeat step 41 2 more times.
42| Aspirate PBS and add 150˜200 μl of blocking buffer. Incubate the plate at room temperature for 1 h.
43| Aspirate the blocking buffer and add 150˜200 μl of the antibody dilution buffer, containing the desired primary antibodies. Incubate the plate overnight at 4° C.
If the surface of the wells is not covered fully by the antibody dilution buffer, increase the antibody solution amount from 150 to 200 μl. If one stain with biotinylated-LTL, it is necessary to use a streptavidin/biotin blocking kit.
44| Place the plate at room temperature, and wait for 15 min. Aspirate the antibody solution and add 500 μl PBS. Wash out the remnant of antibody.
45| Repeat washing with 500 μl PBS 2 more times.
46| Aspirate PBS and add 150˜200 μl of the antibody dilution buffer, containing the desired secondary antibodies. Incubate the plate at room temperature for 1 h in a dark drawer.
47| Aspirate the secondary antibody-containing buffer and add 500 μl PBS. Wash out the remnant of the antibody.
48| Repeat washing with 500 μl PBS 2 more times.
49| Aspirate PBS and add DAPI (1:5000) in PBS.
50| Observe the samples under a immunofluorescence microscope. One don't need to wash out DAPI. If one need quantification of SIX2+ cells with immunocytochemistry, use standard image software such as ImageJ. Alternatively, use flow cytometry for SIX2+ cells with the same antibody dilution ratio (1:500) as for immunocytochemistry.

### EXAMPLE 32

### Endpoint Analysis (Step 40-81)

Typically, nephron structures are visible after 3˜5 days of culture after the “renal vesicle stage” in 2D culture (FIG. 15). In 2D culture, segmented-nephron structures can be analyzed by standard immunocytochemistry for markers of podocytes (PODXL), proximal tubules (lotus tetraglonolobus lectin (LTL)), loops of Henle (cadherin 1 (CDH1), uromodulin (UMOD)), and distal tubules (CDH1) (step 40-51) (FIG. 16b). In 3D culture, frozen sections can be made by standard protocols, and nephron structures can be analyzed by immunohistochemistry (step 52-67) (FIG. 16c). Alternatively, whole mount staining can also be performed, which enables the observation of 3D nephron structures with confocal microscopy (step 68-81) (FIG. 16d). Glomerular and tubular structures can occasionally be recognized with bright field imaging near the surface of the organoids (FIG. 16e).

### EXMPLE 33

### Endpoint Analysis (3 Dimensional, Frozen Sections) TIMING 2 d

51| Transfer the kidney organoids into eppendorf tubes using a pipette with wide tips (simply cut the tip with scissors).
52| Gently aspirate the medium with a 200 μl pipette and add 500 μl 4% PFA. Incubate the organoids at room temperature for 1 h.
53| Gently aspirate 4% PFA with a 200 μl pipette and add 1 ml PBS. Wash the organoids.
54| Repeat washing with 1 ml PBS 2 more times.
55| Gently aspirate PBS with pipette and add 500 μl 30% (w/w) sucrose. Incubate the organoids overnight at 4° C.
56| Transfer the organoids into the center of cryomolds with the wide-tipped pipette and gently aspirate 30% sucrose with a 200 μl pipette. Add OCT compound circumferentially around the periphery of the cryomolds.
57| Freeze the samples with liquid nitrogen and acetone.
58| Cut frozen sections of 5˜10 μm thickness using a cryostat and mount the sections on glass slides.
59| Serially wash off the OCT compound using 3 Coplin jars filled with PBS.
60| Place ˜30 μl of blocking buffer on each section after circling the section with a hydrophobic pen. Incubate the slides at room temperature for 1 h.
If one stain with biotinylated-LTL, it is necessary to use a streptavidin/biotin blocking kit.
61| Aspirate the blocking buffer and add ˜30 μl of antibody dilution buffer, containing desired primary antibodies. Incubate the slides at room temperature for 1˜2 h.
62| Aspirate antibody dilution buffer and wash with PBS 3 times.
63| Aspirate PBS and place ˜30 μl of antibody dilution buffer, containing desired secondary antibodies. Incubate the slides at room temperature for 1 h in a dark drawer.
64| Aspirate antibody dilution buffer and wash with PBS 3 times.
65| Seal the sections using a cover glass slide and Vectashield mounting medium with DAPI.
66| Observe the samples under a immunofluorescence microscope or by confocal microscopy.

### EXAMPLE 34

### Endpoint Analysis (3 Dimensional, Whole Mount Staining) TIMING 3 d

67| Transfer the kidney organoids into eppendorf tubes using a pipette with wide tips (simply cut the tip with scissors).
68| Aspirate the medium with pipette gently and add 500 μl 4% PFA. Incubate the organoids at room temperature for 1 h.
69| Aspirate 4% PFA and add 1 ml PBS. Wash the organoids.
70| Repeat washing with 1 ml PBS 2 more times.
71| Gently aspirate PBS with a 200 μl pipette and add 200 μl blocking buffer. Incubate the organoids at room temperature for 1 h.
72| Gently aspirate blocking buffer with a 200 μl pipette and add 200 μl antibody dilution buffer, containing desired primary antibodies. Incubate the organoids overnight at 4° C.
73| Gently aspirate antibody-containing solution with a 200 μl pipette and add 1 ml PBS. Incubate the organoids at room temperature for 1 h.
74| Gently aspirate PBS with a 200 μl pipette and add 1 ml PBS. Incubate the organoids at room temperature for 1 h.
75| Gently aspirate PBS with a 200 μl pipette and add 1 ml PBS. Incubate the organoid overnight at 4° C.
76| Gently aspirate PBS with pipette and add 200 μl antibody dilution buffer, containing desired secondary antibodies. Incubate the organoids at room temperature for 1 h.
77| Gently aspirate secondary antibody-containing buffer with a 200 μl pipette and wash the organoids with 1 ml PBS 3 times.
78| Gently aspirate PBS with a 200 μl pipette and add 200 μl of DAPI (1:5000) in PBS. Incubate the organoids at room temperature for 1 h.
79| Transfer the organoids onto glass slides with a pipette with wide tips. Aspirate DAPI solution and seal with Vectashield and a cover glass slide.
80| Observe the samples under a confocal microscope.

### EXAMPLE 35

### Nephrotoxicity Assay with Cisplatin TIMING 2 d

81| Prepare kidney organoids in either 2D or 3D culture after at least 21 days of the differentiation.
82| Prepare the basic differentiation medium supplemented with cisplatin 5 μM (1:1000 dilution) or sterile water as a negative control. Required volume for 1 well of 3D culture or 2D culture is 200 μl or 500 μl respectively.
Make sure there is no precipitation of cisplatin in the aliquot. If one see precipitation when the aliquot is taken out from the freezer, warm up the aliquot in a water bath at 37° C. until cisplatin is completely dissolved.
83| Gently aspirate the medium and add 200 μl (3D) or 500 μl (2D) of the basic differentiation medium supplemented with cisplatin 5 μM or sterile water.
Aspiration of kidney organoids would damage tubules, which might result in induction of KIM-1 expression. Be careful not to aspirate kidney organoids.
84| Culture the cells at 37° C. in a 5% CO₂ incubator for one day. Harvest samples for analyses.

### EXAMPLE 36

### Nephrotoxicity Assay with Cisplatin (Step 90-91)

There are a variety of possible applications using NPCs and kidney organoids. As an example of one of these applications, the Inventors show a nephrotoxicity assay with cisplatin, a known nephrotoxicant (FIG. 17). Once nephron structures formed in kidney organoids (day 21˜), one can treat the organoids with agents to interrogate nephrotoxicity. The Inventors demonstrate the response to cisplatin, a known nephrotoxicant. The Inventors used KIM-1 staining to detect proximal tubular injury.

### EXAMPLE 37

### Limitations

The differentiation efficiency is affected by the variability intrinsic to hPSC lines. The Inventors have clarified how to adjust the protocol for different cell lines grown initially in different culture conditions, reflecting the Inventors' experience with 2 different culture media and multiple hPSC lines. The Inventors recommend use of H9 and ReproFF2, as the simplest methods to achieve high differentiation efficiency. The Inventors believe that the the Inventors' adjustment method will enable researchers in different environments to generate NPCs and kidney organoids with different culture systems and different cell lines.

Another limitation of the Inventors' protocols is that the cells in the interstitial space of kidney organoids were not well characterized in the Inventors' original study because of lack of validated antibodies in human kidney samples and definitive morphological characteristics. Those cells were presumably derived from SIX2-negative population which accounted for 10˜20% of cells at the NPC stage, and could be collecting duct cells, pericytes, endothelial cells, smooth muscle cells, fibroblasts or others according to published studies. The Inventors' recent results showed CDH1+AQP2+ tubules (characteristic of connecting tubules/collecting ducts) and PDGFRIβ+ (characteristic of pericytes), endomucin+(characteristic of endothelial cells), or α-SMA+ (characteristic of myofibroblasts) interstitial cells in the organoids (FIG. 18); however, further definitive analyses of these cells are ongoing. The Inventors therefore hope that further studies from us and other investigators will elucidate the characteristics and state of cell types in the interstitial space. The Inventors believe that the organoid system derived from the Inventors' protocols is appropriate to study the interactions between nephron epithelial cells and interstitial cells in a human in vitro model system which recapitulates the complexities of these interactions in the intact organ. In this way the Inventors hope to unlock new insight into processes such as kidney fibrosis, a fundamental process resulting in chronic kidney disease.

The various methods and techniques described above provide a number of ways to carry out the invention. Of course, it is to be understood that not necessarily all objectives or advantages described may be achieved in accordance with any particular embodiment described herein. Thus, for example, those skilled in the art will recognize that the methods can be performed in a manner that achieves or optimizes one advantage or group of advantages as taught herein without necessarily achieving other objectives or advantages as may be taught or suggested herein. A variety of advantageous and disadvantageous alternatives are mentioned herein. It is to be understood that some preferred embodiments specifically include one, another, or several advantageous features, while others specifically exclude one, another, or several disadvantageous features, while still others specifically mitigate a present disadvantageous feature by inclusion of one, another, or several advantageous features.

Furthermore, the skilled artisan will recognize the applicability of various features from different embodiments. Similarly, the various elements, features and steps discussed above, as well as other known equivalents for each such element, feature or step, can be mixed and matched by one of ordinary skill in this art to perform methods in accordance with principles described herein. Among the various elements, features, and steps some will be specifically included and others specifically excluded in diverse embodiments.

Although the invention has been disclosed in the context of certain embodiments and examples, it will be understood by those skilled in the art that the embodiments of the invention extend beyond the specifically disclosed embodiments to other alternative embodiments and/or uses and modifications and equivalents thereof.

Many variations and alternative elements have been disclosed in embodiments of the present invention. Still further variations and alternate elements will be apparent to one of skill in the art. Among these variations, without limitation, are sources of generating intermediate mesoderm, metanephric mesenchyme, nephronic progenitor cells, methods of generating intermediate mesoderm, metanephric mesenchyme, nephronic progenitor cells, and the particular use of the products created through the teachings of the invention. Various embodiments of the invention can specifically include or exclude any of these variations or elements.

In some embodiments, the numbers expressing quantities of ingredients, properties such as concentration, reaction conditions, and so forth, used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements.

In some embodiments, the terms “a” and “an” and “the” and similar references used in the context of describing a particular embodiment of the invention (especially in the context of certain of the following claims) can be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

Preferred embodiments of this invention are described herein, including the best mode known to the inventors for carrying out the invention. Variations on those preferred embodiments will become apparent to those of ordinary skill in the art upon reading the foregoing description. It is contemplated that skilled artisans can employ such variations as appropriate, and the invention can be practiced otherwise than specifically described herein. Accordingly, many embodiments of this invention include all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents and printed publications throughout this specification. Each of the above cited references and printed publications are herein individually incorporated by reference in their entirety.

In closing, it is to be understood that the embodiments of the invention disclosed herein are illustrative of the principles of the present invention. Other modifications that can be employed can be within the scope of the invention. Thus, by way of example, but not of limitation, alternative configurations of the present invention can be utilized in accordance with the teachings herein. Accordingly, embodiments of the present invention are not limited to that precisely as shown and described.

1. A method for generating metanephric mesenchyme, comprising:
providing a quantity of human pluripotent stem cells (“hPSCs”);
generating late primitive streak cells;
inducing formation of posterior intermediate mesoderm cells; and
differentiating into metanephric mesenchyme cells.

2. The method of claim 1, wherein the human pluripotent stem cells are human embryonic stem cells (“hESCs”) or human induced pluripotent stem cells (“hiPSCs).

3. (canceled)

4. The method of claim 1, wherein generating late primitive streak cells comprises culturing in CHIR99021 for about 3-5 days.

5. The method of claim 4, further comprising addition of noggin.

6. The method of claim 1, wherein inducing formation of posterior intermediate mesoderm cells comprises culturing in the presence of activin for about 2-4 days.

7. The method of claim 1, wherein differentiating into metanephric mesenchyme cells comprises addition of FGF9.

8. (canceled)

9. The method of claim 1, wherein the late primitive streak cells express one or more of: T and TBX.

10. The method of claim 1, wherein the posterior intermediate mesoderm cells express one or more of: WT1 and HOXD11.

11. The method of claim 1, wherein metanephric mesenchyme lineage cells express one or more of: SIX2, SALL1, WT1, and PAX2.

12. (canceled)

13. The method of claim 1, wherein differentiation into metanephric mesenchyme cells is at least 50% efficient.

14. (canceled)

15. A composition of metanephric mesenchyme cells generated by the method of claim 1.

16. (canceled)

17. A method of generating kidney organoids, comprising:
providing a quantity of nephron progenitor cells (“NPCs”); and
culturing the NPCs in a suspension culture for about 11 days.

18. The method of claim 17, further comprising addition of one or more of: CHIR99021 and FGF9.

19. The method of claim 17, wherein the kidney organoids comprise one or more cell types selected from the group consisting of: podocyte-like cells, proximal tubules, descending limbs of Henle, thick ascending limbs of Hendle, and distal convoluted tubules.

20. The method of claim 19, wherein the podocyte-like cells express one or more of: NPHS1+, PODXL+, and WT1+.

21. The method of claim 19, wherein the proximal tubules express one or more of: LTL+ and AQP1+.

22. The method of claim 19, wherein the descending limbs of Henle express one or more of: CDH1+ and AQP1+.

23. The method of claim 19, wherein the thick ascending limbs of Henle express one or more of CDH1+ and UMOD+.

24. The method of claim 19, wherein the distal convoluted tubules express one or more of CDH1+UMOD−.

25. The method of claim 17, wherein the NPCs are derived from human pluripotent stem cells (“hPSCs”).

26. The method of claim 25, wherein the hPSCs are derived from a patient suffering from a disease mutation and/or the hPSCs have been genomically edited using CRISPR.

27.-28. (canceled)
